(19) United States
(12) Patent Application Publication
(10) Pub. No.: US 2013/0224839 A1
(43) Pub. Date: Aug. 29, 2013

(54) Recombinant Microorganism and Methods of Production Thereof

(71) Applicant: LanzaTech New Zealand Limited, (US)

(72) Inventors: Michael Koepke, Auckland (NZ); FungMin Liew, Auckland (NZ); Sean Dennis Simpson, Auckland (NZ)

(21) Appl. No.: 13/888,098

(22) Filed: May 6, 2013

Related U.S. Application Data
(63) Continuation-in-part of application No. 13/073,069, filed on Mar. 28, 2011, now abandoned.
(60) Provisional application No. 61/438,805, filed on Feb. 2, 2011.

(51) Int. Cl. C12N 15/74

(52) U.S. Cl. CPC C12N 15/74
USPC 435/252.3

(57) ABSTRACT

The invention relates to a recombinant carboxydotrophic acetogenic microorganism capable of producing one or more products by fermentation of a substrate comprising CO, wherein the microorganism has an increased tolerance to ethanol versus a parental carboxydotrophic acetogenic microorganism. The invention also provides, inter alia, methods for the production of ethanol and one or more other products from a substrate comprising CO using the recombinant carboxydotrophic acetogenic microorganism.

### CROSS REFERENCE TO RELATED APPLICATIONS

This application is a continuation-in-part of copending U.S. non provisional application Ser. No. 13/073,069 filed on Mar. 28, 2011 which claims the priority of U.S. provisional application 61/438,805 filed on Feb. 2, 2011 the contents of each application is herein incorporated by reference in their entirety.

### FIELD OF THE INVENTION

The present invention relates to a recombinant carboxydotrophic acetogenic microorganism with increased tolerance to ethanol.

### BACKGROUND OF THE INVENTION

The growth of most bacteria is affected by relatively low concentrations of alcohols or solvents such as ethanol or butanol. However, the biotechnological production of alcohols is of great interest, for example for use as biofuels. The low natural tolerance of bacteria towards alcohols sets a physical limit for alcohol production, if the alcohol is not removed continuously. The removal of alcohol on the other hand gets far more energy intense and expensive the lower the alcohol concentration (beer strength) (Madson P W: Ethanol distillation: the fundamentals. In: Jaques K A, Lyons T P, Kelsall D R (Eds.): The Alcohol Textbook. 4^{th} edition. 2003, Nottingham University Press: 319-336).

Thus the high toxicity of ethanol and butanol for microorganisms is one of the major problems in bacterial ethanol fermentations as well as the ABE (acetone-butanol-ethanol) fermentation. Only few bacteria, such as some Zymomonas mobilis or Lactococcus strains can tolerate more than 10% ethanol, while the majority of bacteria can only tolerate a maximum of 4-7% ethanol. Butanol is even more toxic for bacterial cells, hardly exceeding levels greater than 1.5-2.5% butanol, while mixtures of different alcohols were shown to act in a synergistic way. Two species of the biotechnologically important genus Clostridium analyzed for alcohol tolerance were shown to tolerate only moderate levels of up to 4-5% or 40-50 g/l ethanol (Rani K S, Seenayya G: High ethanol tolerance of new isolates of Clostridium thermocellum strains SS21 and SS22. World J Microbiol Biotechnol 1999, 2: 173-178; Baskaran S, Ahn H J, Lynd L R: Investigation of the Ethanol Tolerance of Clostridium thermosaccharolyticum in Continuous Culture. Biotechnol Prog 1995, 3: 276-281) or around 1.5% butanol (Liu S, Qureshi N: How microbes tolerate ethanol and butanol. New Biotechnol 2009, 3-4: 117-121). However, most natural isolates of bacteria shown to have high alcohol tolerance aren't suited as production strains, as they only produce low alcohol yields, or even live on alcohols as carbon source. Thus, there is a need to improve current production strains for higher alcohol tolerance.

Increased butanol levels have been shown to elicit a response similar to a heat shock. Several heat shock stress proteins/chaperons such as ClpB, ClpC, ClpP, DnaK, DnaJ, GreA, GroES, GroEL, GrpE, Hsp18, Hsp90, HtrA, Map, TufA, TufB, or YacI were found to upregulated, both on genetic (Alsaker K V, Paredes C, Papoutsakis E T: Metabolite stress and tolerance in the production of biofuels and chemicals: gene-expression-based systems analysis of butanol, butyrate, and acetate stresses in the anaerobe Clostridium acetobutylicum. Biotechnol Bioeng 2010, 105: 1131-1147; Tomas C A, Beamish J, Papoutsakis E T: Transcriptional Analysis of Butanol Stress and Tolerance in Clostridium acetobutylicum. J Bacteriol 2004, 186: 2006-2018) and protein (Mao S, Luo Y M, Zhang T, Li J, Bao G, Zhu Y, Chen Z, Zhang Y, Li Y, Ma Y: A proteome reference map and comparative proteomic analysis between a wild-type Clostridium acetobutylicum DSM 1731 and a mutant strain with enhanced butanol tolerance and butanol yield. J Proteome Res 2010, 9: 3046-3061) level. Overproduction of Heat shock protein/chaperonin complex GroESL in Clostridium acetobutylicum resulted in a strain which was up to 85% less inhibited by butanol challenge, prolonged metabolism and higher solvent yield compared to the wild-type (Tomas C A, Welker N E, Papoutsakis E T: Overexpression of groESL in Clostridium acetobutylicum results in increased solvent production and tolerance, prolonged metabolism, and changes in the cell's transcriptional program. Appl Environ Microbiol 2003, 69: 4951-49650). The effect of groESL overexpression on ethanol tolerance has not been reported.

In U.S. Pat. No. 6,960,456 Papoutsakis et al describe a recombinant strain of solventogenic Clostridia (Clostridium acetobutylicum) having increased expression of a chaperon for increased resistance to toxic organic substrates. The patentee shows that their Clostridium acetobutylicum has an increased butanol tolerance but produces lower amounts of ethanol versus the wild type organism. No mention is made regarding tolerance to ethanol, nor the effect such chaperons would have on ethanol toxicity.

Clostridia can be divided into three fundamentally different groups (Tracy, Jones, Fast, Indurthi, & Papoutsakis, 2012):

a. Solventogenic clostridia (such as C. acetobutylicum, C. beijerinckii, and C. butyricum)
b. Cellulolytic clostridia (such as C. thermocellum, C. cellulolyticum, and C. phytofermentans)
c. Clostridial acetogens (such as C. ljungdahlii, C. thermoaceticum, and C. carboxidivorans or C. autoethanogenum)

The solventogenic and cellulolytic Clostridia groups are both related in that they utilize carbohydrates via glycolysis and are thus rich in ATP, while Clostridial acetogens utilize gases CO and H₂ via the Wood-Ljungdahl pathway which is scarce in ATP. For the solventogenic bacterium C. acetobutylicum the ATP gain from substrate level phosphorylation is four ATP per molecule of substrate glucose (Jones & Woods, 1986), while the Wood-Ljungdahl pathway requires 1 ATP to activate CO₂. The Tracy et al reference shows that the behavior of solventogenic clostridia and cellulolytic clostridia does not predict behavior in clostridial acetogens.

Schiel and Durre (B. Schiel and P. Dürre, Clostridium, Encyclopedia of Industrial Biotechnology: Bioprocess, Bioseparation and Cell Technology, M. C. Flickinger, editor; John Wiley & Sons, 2010, p. 1-15; DOI: 10.1002/9780470054581.eib236) differentiate between butyrate and butanol fermenting Clostridia and (hom)acetogenic Clostridia.

The GroESL complex is known to be highly energy intense requiring 7-14 ATP per action, thus folding of a monomeric enzyme by GroESL in vitro requires more than 100 ATP (Martin et al., 1991). In addition, protein biosynthesis of this large complex (7mer GroEL and 14mer GroES) requires further energy. Recent work in E. coli (Zingaro & Terry Papoutsakis, 2012) “suggest a complex pattern of growth inhibition and differential protection by GroESL overexpression depending on the specific alcohol molecule”, thus tolerance of butanol is not a predictor of tolerance of ethanol.

It is an object of the invention to overcome one or more of the disadvantages of the prior art, or to at least to provide the public with a useful choice.

### SUMMARY OF THE INVENTION

In a first aspect, the invention provides a recombinant carboxydotrophic acetogenic microorganism capable of producing one or more products by fermentation of a substrate comprising CO, wherein the microorganism has an increased tolerance to ethanol.

In one embodiment, the recombinant carboxydotrophic acetogenic microorganism is tolerant of ethanol concentrations of at least approximately 5.5% by weight of fermentation broth (i.e. 55 g ethanol/L of fermentation broth). In one particular embodiment, the recombinant carboxydotrophic acetogenic microorganism is tolerant of ethanol concentrations of at least approximately 6% by weight of fermentation broth.

Preferably, the recombinant carboxydotrophic acetogenic microorganism is adapted to express, and in one particular embodiment over-express, one or more enzymes adapted to increase tolerance to ethanol.

In one embodiment the one or more enzymes are chosen from the group consisting of stress proteins and chaperones.

In one embodiment, the one or more enzymes are chosen from the group consisting of:

protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES), Cpn60 chaperonin (GroEL), heat shock protein (GrpE), heat shock protein (Hsp18), heat shock protein (Hsp90), membrane bound serine protease (HtrA), methionine aminopeptidase (Map), protein chain elongation factor (TufA), protein chain elongation factor (TufB), or Arginine kinase related enzyme (YacI).

In one embodiment, the one or more enzymes are GroES and GroEL.

In one embodiment, the recombinant carboxydotrophic acetogenic microorganism comprises one or more exogenous nucleic acids adapted to increase expression of one or more nucleic acids native to the microorganism and which encode one or more enzymes referred to herein before. In one embodiment, the one or more exogenous nucleic acid adapted to increase expression is a promoter. In one embodiment, the promoter is a constitutive promoter. In one particular embodiment, the exogenous promoter is a pyruvate:ferredoxin oxidoreductase promoter. In one particular embodiment, the promoter has the nucleic acid sequence of SEQ_ID NO. 5 or a functionally equivalent variant thereof.

In one embodiment, the recombinant carboxydotrophic acetogenic microorganism comprises one or more exogenous nucleic acids encoding and adapted to express the one or more enzymes referred to herein before.

Preferably, the recombinant carboxydotrophic acetogenic microorganism comprises one or more exogenous nucleic acids encoding each of GroES (SEQ ID No. 1) and GroEL (SEQ_ID NO. 2). In one particular embodiment nucleic acids encoding each of GroES and GroEL are defined by SEQ_ID NO. 3 and 4 or a functionally equivalent variant thereof.

In one embodiment, the recombinant carboxydotrophic acetogenic microorganism comprises a nucleic acid construct or vector encoding the one or more enzymes referred to hereinbefore. In one particular embodiment, the construct/vector encodes one or both, and preferably both, of GroES and GroEL.

In one embodiment, the nucleic acid construct/vector further comprises an exogenous promoter. In one particular embodiment, the exogenous promoter is a pyruvate:ferredoxin oxidoreductase promoter. In one particular embodiment, the promoter has the nucleic acid sequence of SEQ_ID NO. 5 or a functionally equivalent variant thereof.

In one embodiment, the nucleic acid construct/vector further comprises an exogenous promoter. In one particular embodiment, the exogenous promoter is a Wood-Ljungdahl cluster promoter. In one particular embodiment, the promoter has the nucleic acid sequence of SEQ_ID NO. 25 or a functionally equivalent variant thereof.

In one embodiment, the recombinant carboxydotrophic acetogenic microorganism is selected from the group consisting of Clostridium autoethanogenum, Clostridium ljungdahlii, Clostridium ragsdalei, Clostridium carboxidivorans, Clostridium drakei, Clostridium scatologenes, Butyribacterium limosum, Butyribacterium methylotrophicum, Acetobacterium woodii, Alkalibaculum bacchii, Blautia producta, Eubacterium limosum, Moorella thermoacetica, Moorella thermautotrophica, Oxobacter pfennigii, and Thermoanaerobacter kiuvi.

In one particular embodiment, the microorganism is Clostridium autoethanogenum DSM23693.

In a second embodiment, the invention provides a nucleic acid encoding one or more enzymes, preferably two or more enzymes, which when expressed in a carboxydotrophic acetogenic microorganism result in the microorganism having an increased tolerance to ethanol. In one embodiment the enzyme is chosen from the group consisting of stress proteins and chaperones.

In one particular embodiment, the nucleic acid encodes one or more enzyme chosen from the group consisting of ClpB, ClpC, ClpP, DnaK, DnaJ, GreA, GroES, GroEL, GrpE, Hsp18, Hsp90, HtrA, Map, TufA, TufB, or YacI, or functionally equivalent variants thereof, in any order.

In one embodiment, the nucleic acid encodes both GroES and GroEL. In one particular embodiment, the nucleic acid comprises SEQ_ID No 3 and 4, or functionally equivalent variants thereof, in any order. In one embodiment, the nucleic acid comprises SEQ ID_NO. 12, or a functionally equivalent variant thereof.

Preferably, the nucleic acids of this aspect of the invention further comprise a promoter. Preferably, the promoter is a pyruvate:ferredoxin oxidoreductase promoter. In one particular embodiment, the promoter has the nucleic acid sequence of SEQ_ID NO. 5 or a functionally equivalent variant thereof.

In another aspect, the invention provides a nucleic acid construct or vector comprising a nucleic acid of the second aspect of the invention.

In another aspect, the invention provides a nucleic acid consisting of the sequence of any one of SEQ ID NO.s 6, 7, 8, 9, 10, 11, 29, 30, 31, 32, 33 and 34.

In a third aspect, the invention provides an expression construct or vector comprising a nucleic acid sequence encoding one or more enzymes, preferably two or more enzymes, wherein the construct/vector, when expressed in a carboxydotrophic acetogenic microorganism, results in the microorganism having an increased tolerance to ethanol.

Preferably, the enzymes are chosen from the group consisting of stress proteins and chaperones.

In one embodiment, the construct/vector comprises a nucleic acid sequence encoding two or more of the enzymes chosen from the group consisting ClpB, ClpC, ClpP, DnaK, DnaJ, GreA, GroES, GroEL, GrpE, Hsp18, Hsp90, HtrA, Map, TufA, TufB, or YacI, in any order.

Preferably, the construct/vector comprises nucleic acid sequences encoding each of GroES (SEQ ID No. 1) and GroEL (SEQ_ID NO. 2). In one particular embodiment, the construct/vector comprises the nucleic acid sequences SEQ_ID NO. 3 and 4 or a functionally equivalent variant thereof, in any order. In one embodiment, the construct/vector comprises SEQ ID_NO. 12, or a functionally equivalent variant thereof.

Preferably, the expression construct/vector further comprises a promoter. Preferably the promoter is a pyruvate:ferredoxin oxidoreductase promoter. In one particular embodiment, the promoter has the nucleic acid sequence of SEQ_ID NO. 5 or a functionally equivalent variant thereof.

In one particular embodiment, the expression construct/vector is a plasmid. In one embodiment, the expression plasmid has the nucleotide sequence SEQ ID No. 17.

In another aspect, the invention provides a host cell comprising one or more nucleic acids of the invention.

In a fourth aspect, the invention provides a composition comprising an expression construct/vector as referred to in the third aspect of the invention and a methylation construct/vector.

Preferably, the composition is able to produce a recombinant microorganism which has increased ethanol tolerance.

In one particular embodiment, the expression construct/vector and/or the methylation construct/vector are plasmids.

In a fifth aspect, the invention provides a method of producing a recombinant carboxydotrophic acetogenic microorganism having increased tolerance to ethanol comprising:
a. introduction into a shuttle microorganism of (i) an expression construct/vector of the third aspect of the invention and (ii) a methylation construct/vector comprising a methyltransferase gene;
b. expression of the methyltransferase gene;
c. isolation of one or more constructs/vectors from the shuttle microorganism; and,
d. introduction of at least the expression construct/vector into a destination microorganism.

In one embodiment, the methyltransferase gene of step (b) is expressed constitutively. In another embodiment, expression of the methyltransferase gene of step (b) is induced.

In one embodiment, both the methylation construct/vector and the expression construct/vector are isolated in step (c). In another embodiment, only the expression construct/vector is isolated in step (c).

In one embodiment, only the expression construct/vector is introduced into the destination microorganism. In another embodiment, both the expression construct/vector and the methylation construct/vector are introduced into the destination microorganism.

In a related aspect, the invention provides a method of producing a recombinant microorganism having increased tolerance to ethanol comprising:
a. methylation of an expression construct/vector of the third aspect of the invention in vitro by a methyltransferase;
b. introduction of the expression construct/vector into a destination microorganism.

In a further related aspect, the invention provides a method of producing a recombinant microorganism having increased tolerance to ethanol comprising:
a. introduction into the genome of a shuttle microorganism of a methyltransferase gene
b. introduction of an expression construct/vector of the third aspect of the invention into the shuttle microorganism
c. isolation of one or more constructs/vectors from the shuttle microorganism; and,
d. introduction of at least the expression construct/vector into a destination microorganism.

In a sixth aspect, the invention provides a method for the production of ethanol and/or one or more other products by microbial fermentation comprising fermenting a substrate comprising CO using a recombinant carboxydotrophic acetogenic microorganism of the first aspect of the invention.

The invention also provides a method for reducing the total atmospheric carbon emissions from an industrial process.

In one embodiment the method comprises the steps of:
(a) providing a substrate comprising CO to a bioreactor containing a culture of one or more recombinant carboxydotrophic acetogenic microorganism of the first aspect of the invention; and
(b) anaerobically fermenting the culture in the bioreactor to produce one or more products including ethanol.

In another embodiment the method comprises the steps of:
(a) capturing CO-containing gas produced as a result of the industrial process, before the gas is released into the atmosphere;
(b) the anaerobic fermentation of the CO-containing gas to produce one or more products including ethanol by a culture containing one or more recombinant carboxydotrophic acetogenic microorganism of the first aspect of the invention.

In one embodiment, the recombinant carboxydotrophic microorganism is tolerant of ethanol concentration in the fermentation broth of at least about 5.5% by weight. In another embodiment, the recombinant carboxydotrophic microorganism is tolerant of ethanol concentration in the fermentation broth of at least about 6% by weight. In a further embodiment the recombinant carboxydotrophic microorganism is tolerant of ethanol concentration in the fermentation broth of from about 3 to about 15% by weight. In another embodiment the recombinant carboxydotrophic microorganism is tolerant of ethanol concentration in the fermentation broth of from about 5.5 to about 15% by weight or from about 6% to about 15% by weight or from about 5.5% to about 10% by weight.

In particular embodiments of the method aspects, the recombinant carboxydotrophic acetogenic microorganism is maintained in an aqueous culture medium.

In particular embodiments of the method aspects, the fermentation of the substrate takes place in a bioreactor.

Preferably, the substrate comprising CO is a gaseous substrate comprising CO. In one embodiment, the substrate comprises an industrial waste gas. In certain embodiments, the gas is steel mill waste gas or syngas.

In one embodiment, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume.

In certain embodiments the methods further comprise the step of recovering the one or more products from the fermentation broth.

In a seventh aspect, the invention provides ethanol and/or one or more other product when produced by the method of the sixth aspect.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

These and other aspects of the present invention, which should be considered in all its novel aspects, will become apparent from the following description, which is given by way of example only, with reference to the accompanying figures, in which:

FIG. 1 shows Ethanol tolerance of (a) Clostridium autoethanogenum DSM23693, (b) Clostridium autoethanogenum DSM10061, and (c) Clostridium ljungdahlii DSM13528 in serum bottles.

FIG. 2 shows Expression of the pyruvate:ferredoxin oxidoreductase during a normal batch fermentation run compared to over 200 genes of interest.

FIG. 3 illustrates the DNA sequencing of groESL insert in plasmid pCR-Blunt-GroESL.

FIG. 4 shows a map of the plasmid pMTL85246-GroESL.

FIG. 5 illustrates the DNA sequencing alignment of P_{pfor} and groESL insert in plasmid pMTL85246-GroESL.

FIG. 6 shows the methylation plasmid.

FIG. 7 shows detection of ermB (400 bp) and groESL (2 kbp) from PCR of plasmid isolated from transformed C. autoethanogenum DSM23693. Ladder=1 KB Plus DNA ladder (Invitrogen); 1=ermB from non-template control; 2=ermB from plasmid isolated from C. autoethanogenum; 3=ermB from original plasmid pMTL 85246-GroESL (as positive control); 4=groESL from non-template control; 5=groESL from plasmid isolated from C. autoethanogenum; 6=groESL from original plasmid pMTL 85246-GroESL (as positive control).

FIG. 8 illustrates an ethanol challenge experiment with C. autoethanogenum DSM23693 wild-type (WT) and transformed strain carrying plasmid pMTL 85246-GroESL.

FIG. 9: Table of exemplary information for enzymes of use in the invention. The protein accession number is followed by the gene ID (GenBank) for each microorganism listed.

FIG. 10. Plasmid map of pMTL83156-grpE-dnaK-dnaJ.

FIG. 11. Plasmid map of pMTL83157.

FIG. 12. Sequencing of promoter P_{pfor} and grpE-dnaK-dnaJ insert in plasmid pMTL83156-grpE-dnaK-dnaJ.

FIG. 13. Gel electrophoresis showing the presence of introduced plasmids by PCR of catP and repH (1500 bp). L=NEB 2-Log DNA ladder; NTC=no template control; GC1=C. autoethanogenum DSM10061 WT genomic DNA control; GC2=C. ljungdahlii DSM13528WT genomic DNA control; PC=plasmid control (pMTL83157); 1-4=pMTL83156-groESL from C. autoethanogenum DSM10061; 5-8=pMTL83156-groESL from C. ljungdahlii; 9-12=pMTL83156-grpE-dnaK-dnaJ from C. ljungdahlii DSM13583; 13-16=pMTL83156-grpE-dnaK-dnaJ from C. autoethanogenum DSM10061; 17-20=pMTL83157 from C. autoethanogenum DSM10061.

FIG. 14. Gel electrophoresis showing the expected restriction digest (PmeI+AscI) bands from rescued plasmids. L=NEB 2-Log DNA ladder; 1-4=pMTL83156-groESL from C. autoethanogenum DSM10061; 5-8=pMTL83156-groESL from C. ljungdahlii DSM13583; 9-11=pMTL83156-grpE-dnaK-dnaJ from C. ljungdahlii DSM13583; 12-25=pMTL83157 from C. ljungdahlii DSM13583; 16-19=pMTL83156-grpE-dnaK-dnaJ from C. autoethanogenum DSM10061; 20-23=pMTL83157 from C. autoethanogenum DSM10061.

FIG. 15. Effect of ethanol on growth of C. autoethanogenum DSM10061 with plasmid control (pMTL83157) and grpE-dnaKJ expression plasmid after 40 hours of growth.

FIG. 16. Over-expression of groESL enhances tolerance towards ethanol at in Clostridium autoethanogenum DSM10061 (a) under autotrophic conditions in PETC medium and (b) under heterotrophic conditions in MMYF medium. Symbols: a) Dark grey diamonds and light grey squares=2 independent clones of C. autoethanogenum, Triangles=wild-type (WT); b) Light grey diamond=pMTL83157 plasmid control, Dark grey square=pMTL83156-groESL;

FIG. 17. Over-expression of groESL and grpE-dnaK-dnaJ enhances tolerance towards ethanol at (A) 5 g/L; (B) 10 g/L; (C) 25 g/L; and (D) 50 g/L, relative to plasmid control in Clostridium ljungdahlii DSM13583. Light Grey diamond=pMTL83157 plasmid control; Dark Grey square=pMTL83156-groESL; Black triangle=pMTL83156-grpE-dnaK-dnaJ. Anaerobic ethanol was administered at 12 hour post inoculation.

FIG. 18. Effect of promoter sequence for heterologous expression of groESL on enhancing tolerance towards ethanol at in Clostridium ljungdahlii DSM83157: Light grey squares=wild-type (WT); Grey triangles=pMTL83156-groESL (pyruvate:ferredoxin oxidoreductase promoter), Dark grey diamonds=pMTL83155-groESL (phosphotransacetylase promoter);

FIG. 19: Over-expression of grpE-dnaK-dnaJ enhances tolerance towards ethanol at 5 g/L, 10 g/L and 25 g/L relative to plasmid control in Clostridium autoethanogenum DSM10061 at 102 hour post inoculation. White column=pMTL83157 plasmid control; Grey column=pMTL83156-grpE-dnaK-dnaJ transformant. % inhibition represents the % reduction in OD₆₀₀ relative to unchallenged culture. Anaerobic ethanol was administered at 12 hour post inoculation.

FIG. 20: Plasmid map of pMTL83156-grpE-dnaK-dnaJ-P_{WL}-groESL.

### DETAILED DESCRIPTION OF THE INVENTION

The following is a description of the present invention, including preferred embodiments thereof, given in general terms. The invention is further elucidated from the disclosure given under the heading “Examples” herein below, which provides experimental data supporting the invention, specific examples of various aspects of the invention, and means of performing the invention.

The invention provides a recombinant carboxydotrophic acetogenic microorganism capable of producing ethanol or, ethanol and one or more other products, by fermentation of a substrate comprising CO, wherein the recombinant carboxydotrophic acetogenic microorganisms has an increased tolerance to ethanol.

Solvents and alcohols are often toxic to microorganisms, even at very low concentrations. This can increase costs and limit the commercial viability of methods for the production of alcohols and other products by bacterial fermentation. The inventors have developed recombinant carboxydotrophic acetogenic microorganisms which surprisingly have increased ethanol tolerance and thus may be used to improve efficiencies of the production of ethanol and/or other products by fermentation of substrates comprising CO.

### DEFINITIONS

As referred to herein, a “fermentation broth” is a culture medium comprising at least a nutrient media and bacterial cells.

As referred to herein, a shuttle microorganism is a microorganism in which a methyltransferase enzyme is expressed and is distinct from the destination microorganism.

As referred to herein, a destination microorganism is a microorganism in which the genes included on an expression construct/vector are expressed and is distinct from the shuttle microorganism.

The term “main fermentation product” is intended to mean the one fermentation product which is produced in the highest concentration and/or yield.

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated ethanol concentrations, the volume of desired product (such as alcohols) produced per volume of substrate consumed, the rate of production or level of production of the desired product, and the relative proportion of the desired product produced compared with other by-products of the fermentation.

“Increased tolerance to ethanol” and like terms should be taken to mean that the recombinant carboxydotrophic acetogenic microorganism has a higher tolerance to ethanol as compared to a parental carboxydotrophic acetogenic microorganism. Tolerance may be measured in terms of the survival of a microorganism or population of microorganisms, the growth rate of a microorganism or population of microorganisms and/or the rate of production of one or more products by a microorganism or population of microorganisms in the presence of ethanol. In one particular embodiment of the invention, it is measured in terms of the ability of a microorganism or population of microorganisms to grow in the presence of ethanol concentrations which are typically toxic to the parental microorganism.

The phrase “substrate comprising carbon monoxide” and like terms should be understood to include any substrate in which carbon monoxide is available to one or more strains of bacteria for growth and/or fermentation, for example.

The phrase “gaseous substrate comprising carbon monoxide” and like phrases and terms includes any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H2:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H2, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

In the description which follows, embodiments of the invention are described in terms of delivering and fermenting a “gaseous substrate containing CO”. However, it should be appreciated that the gaseous substrate may be provided in alternative forms. For example, the gaseous substrate containing CO may be provided dissolved in a liquid. Essentially, a liquid is saturated with a carbon monoxide containing gas and then that liquid is added to the bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used. By way of further example, the gaseous substrate containing CO may be adsorbed onto a solid support. Such alternative methods are encompassed by use of the term “substrate containing CO” and the like.

In particular embodiments of the invention, the CO-containing gaseous substrate is an industrial off or waste gas. “Industrial waste or off gases” should be taken broadly to include any gases comprising CO produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. As is described herein after, in some embodiments the bioreactor may comprise a first growth reactor and a second fermentation reactor. As such, when referring to the addition of substrate to the bioreactor or fermentation reaction it should be understood to include addition to either or both of these reactors where appropriate.

When used in relation to the products of a fermentation in accordance with the invention “one or more other products” is intended to include acetate and 2,3-butanediol, for example. It should be appreciated that the methods of the invention are applicable to methods intended for the production and recovery of products other than ethanol, but where ethanol is produced as a by-product and may have an impact on the efficiency of growth of and production by one or more microorganisms.

The term “acetate” includes both acetate salt alone and a mixture of molecular or free acetic acid and acetate salt, such as the mixture of acetate salt and free acetic acid present in a fermentation broth as described herein. The ratio of molecular acetic acid to acetate in the fermentation broth is dependent upon the pH of the system.

“Exogenous nucleic acids” are nucleic acids which originate outside of the microorganism to which they are introduced. Exogenous nucleic acids may be derived from any appropriate source, including, but not limited to, the microorganism to which they are to be introduced, strains or species of microorganisms which differ from the organism to which they are to be introduced, or they may be artificially or recombinantly created. In one embodiment, the exogenous nucleic acids represent nucleic acid sequences naturally present within the microorganism to which they are to be introduced, and they are introduced to increase expression of or over-express a particular gene (for example, by increasing the copy number of the sequence (for example a gene). In another embodiment, the exogenous nucleic acids represent nucleic acid sequences not naturally present within the microorganism to which they are to be introduced and allow for the expression of a product not naturally present within the microorganism or increased expression of a gene native to the microorganism (for example in the case of introduction of a regulatory element such as a promoter). The exogenous nucleic acid may be adapted to integrate into the genome of the microorganism to which it is to be introduced or to remain in an extra-chromosomal state.

It should be appreciated that the invention may be practised using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants”. By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein. These include homologous genes in species such as Escherichia coli, Bacillus subtilis, Clostridium acetobutylicum, Clostridium ljungdahlii, Clostridium carboxidivorans could be used, details of which are publicly available on websites such as Genbank or NCBI. The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the invention may be practised using polypeptides whose sequence varies from the amino acid sequences specifically exemplified herein. These variants may be referred to herein as “functionally equivalent variants”. A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

“Substantially the same function” as used herein is intended to mean that the nucleic acid or polypeptide is able to perform the function of the nucleic acid or polypeptide of which it is a variant. For example, a variant of an enzyme of the invention will be able to catalyse the same reaction as that enzyme. However, it should not be taken to mean that the variant has the same level of activity as the polypeptide or nucleic acid of which it is a variant.

One may assess whether a functionally equivalent variant has substantially the same function as the nucleic acid or polypeptide of which it is a variant using any number of known methods. However, by way of example, the methods outlined in Zietkiewicz et al (Hsp70 chaperone machine remodels protein aggregates at the initial step of Hsp70-Hsp100-dependent disaggregation, J Biol Chem 2006, 281: 7022-7029), Zzaman et al (The DnaK-DnaJ-GrpE chaperone system activates inert wild-type pi initiator protein of R6K into a form active in replication initiation, J Biol Chem 2004, 279: 50886-50894), Zavilgelsky et al (Role of Hsp70 (DnaK-DnaJ-GrpE) and Hsp100 (ClpA and ClpB) chaperones in refolding and increased thermal stability of bacterial luciferases in Escherichia coli cells, Biochemistry (Mosc) 2002, 67: 986-992), or Konieczny and Liberek (Cooperative action of Escherichia coli ClpB protein and DnaK chaperone in the activation of a replication initiation protein, J Biol Chem 2002, 277: 18483-18488) may be used to assess enzyme activity.

A “stress protein”, as used herein, is intended to include any protein which is expressed in response to stress and includes for example, heat shock proteins, chaperon complexes, transcription elongation factors, proteases, and petidases.

A “chaperone”, as used herein, is intended to include any peptide or protein which is involved in controlling and maintaining the correct folding of proteins and enzymes in their active state, and includes those proteins involved in refolding misfolded and aggregated proteins, for example after exposure to heat or alcohols.

“Over-express”, “over expression” and like terms and phrases when used in relation to the invention should be taken broadly to include any increase in expression of one or more protein as compared to the expression level of the protein of a parental microorganism under the same conditions. It should not be taken to mean that the protein is expressed at any particular level.

A “parental microorganism” is a microorganism used to generate a recombinant microorganism of the invention. The parental microorganism may be one that occurs in nature (ie a wild type microorganism) or one that has been previously modified but which does not express or over-express one or more of the enzymes the subject of the present invention. Accordingly, the recombinant microorganisms of the invention have been modified to express or over-express one or more enzymes that were not expressed or over-expressed in the parental microorganism.

The terms nucleic acid “constructs” or “vectors” and like terms should be taken broadly to include any nucleic acid (including DNA and RNA) suitable for use as a vehicle to transfer genetic material into a cell. The terms should be taken to include plasmids, viruses (including bacteriophage), cosmids and artificial chromosomes. Constructs or vectors may include one or more regulatory elements, an origin of replication, a multicloning site and/or a selectable marker, among other elements, sites and markers. In one particular embodiment, the constructs or vectors are adapted to allow expression of one or more genes encoded by the construct or vector. Nucleic acid constructs or vectors include naked nucleic acids as well as nucleic acids formulated with one or more agents to facilitate delivery to a cell (for example, liposome-conjugated nucleic acid, an organism in which the nucleic acid is contained).

As discussed herein before, the invention provides a recombinant microorganism capable of producing ethanol and one or more other products by fermentation of a substrate comprising CO, wherein the microorganism has an increased tolerance to ethanol.

In one embodiment, the recombinant carboxydotrophic microorganism is tolerant of ethanol concentration in the fermentation broth of at least about 5.5% by weight. In another embodiment, the recombinant carboxydotrophic microorganism is tolerant of ethanol concentration in the fermentation broth of at least about 6% by weight. In a further embodiment the recombinant carboxydotrophic microorganism is tolerant of ethanol concentration in the fermentation broth of from about 3 to about 15% by weight. In another embodiment the recombinant carboxydotrophic microorganism is tolerant of ethanol concentration in the fermentation broth of from about 5.5 to about 15% by weight or from about 6% to about 15% by weight or from about 5.5% to about 10% by weight.

In particular embodiments, the recombinant carboxydotrophic acetogenic microorganism is adapted to express one or more enzyme adapted to increase tolerance to ethanol which are not naturally present in the parental microorganism, or over-express one or more enzyme adapted to increase tolerance to ethanol which are naturally present in the parental microorganism.

The recombinant carboxydotrophic acetogenic microorganism may be adapted to express or over-express the one or more enzymes by any number of recombinant methods including, for example, increasing expression of native genes within the microorganism (for example, by introducing a stronger or constitutive promoter to drive expression of a gene), increasing the copy number of a gene encoding a particular enzyme by introducing exogenous nucleic acids encoding and adapted to express the enzyme, introducing an exogenous nucleic acid encoding and adapted to express an enzyme not naturally present within the parental microorganism.

In certain embodiments, the parental carboxydotrophic acetogenic microorganism may be transformed to provide a combination of increased or over-expression of one or more genes native to the parental carboxydotrophic acetogenic microorganism and introduction of one or more genes not native to the parental microorganism.

In one embodiment the one or more enzymes are chosen from the group consisting of stress proteins and chaperones.

In one embodiment, the one or more enzymes are chosen from the group consisting:

protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES), Cpn60 chaperonin (GroEL), heat shock protein (GrpE), heat shock protein (Hsp18), heat shock protein (Hsp90), membrane bound serine protease (HtrA), methionine aminopeptidase (Map), protein chain elongation factor (TufA), protein chain elongation factor (TufB), or Arginine kinase related enzyme (YacI), and functionally equivalent variants of any one thereof.

Exemplary nucleic acid and amino acid sequence information for the above enzymes are found in GenBank, as outlined in the table in FIG. 30.

In one embodiment, the one or more enzymes are GroES and GroEL.

In one embodiment, the recombinant carboxydotrophic acetogenic microorganism comprises one or more exogenous nucleic acids adapted to increase expression of one or more nucleic acids native to the microorganism and which one or more nucleic acids encode one or more enzymes referred to herein before. In one embodiment, the one or more exogenous nucleic acid adapted to increase expression is a promoter. In one embodiment, the promoter is a constitutive promoter that is preferably highly active under appropriate fermentation conditions. However, inducible promoters may also be employed. In preferred embodiments, the promoter is selected from the group comprising phosphotransacetylase/acetate kinase operon promoter (SEQ_ID No. 24), pyruvate:ferredoxin oxidoreductase (SEQ_ID No. 5), the Wood-Ljungdahl gene cluster (SEQ_ID No 25), Rnf operon (SEQ_ID No 26) or the ATP synthase operon (SEQ_ID No 27). Preferably, the promoter is a pyruvate:ferredoxin oxidoreductase promoter. In one particular embodiment, the promoter has the nucleic acid sequence of SEQ_ID NO. 5 or a functionally equivalent variant thereof. It will be appreciated by those of skill in the art that other promoters which can direct expression, preferably a high level of expression under appropriate fermentation conditions, would be effective as alternatives to the exemplified embodiments.

In one embodiment, the recombinant carboxydotrophic acetogenic microorganism comprises one or more exogenous nucleic acids encoding and adapted to express the one or more enzymes referred to herein before. In one embodiment, the recombinant carboxydotrophic acetogenic microorganism comprises one or more exogenous nucleic acid encoding and adapted to express at least two enzymes adapted to increase tolerance to ethanol. In other embodiments, the recombinant carboxydotrophic acetogenic microorganism comprises one or more exogenous nucleic acid encoding and adapted to express at least 3, at least 4, at least 5 or at least 6 enzymes adapted to increase tolerance to ethanol.

In one embodiment, the recombinant carboxydotrophic acetogenic microorganism comprises one or more exogenous nucleic acid encoding each of GroES and GroEL, or a functionally equivalent variant of either or both. In one particular embodiment nucleic acids encoding each of GroES and GroEL are defined by SEQ_ID NO. 3 and 4 or a functionally equivalent variant thereof. In one embodiment, the recombinant carboxydotrophic acetogenic microorganism comprises a nucleic acid comprises SEQ ID_NO. 12, or a functionally equivalent variant thereof.

In one embodiment, the recombinant carboxydotrophic acetogenic microorganism comprises a nucleic acid construct or vector, for example a plasmid, encoding the one or more enzymes referred to hereinbefore. In one particular embodiment, the construct encodes one or both, and preferably both, of GroES and GroEL. In one embodiment, the construct or vector comprises nucleic acid sequences encoding each of GroES (SEQ ID No. 1) and GroEL (SEQ_ID NO. 2). In one particular embodiment, the vector comprises the nucleic acid sequences SEQ_ID NO. 3 and 4 or a functionally equivalent variant thereof, in any order. In one embodiment, the vector/construct comprises SEQ ID_NO. 12, or a functionally equivalent variant thereof.

In one embodiment, the nucleic acid construct/vector further comprises an exogenous promoter adapted to promote expression of the one or more enzymes encoded by the exogenous nucleic acids.

In one embodiment the promoter is a constitutive promoter that is preferably highly active under appropriate fermentation conditions. However, inducible promoters may also be employed. In preferred embodiments, the promoter is selected from the group comprising phosphotransacetylase/acetate kinase operon promoter (SEQ ID NO. 24), pyruvate:ferredoxin oxidoreductase (SEQ_ID No. 5), the Wood-Ljungdahl gene cluster (SEQ_ID No 25), Rnf operon (SEQ_ID No 26) or the ATP synthase operon ((SEQ_ID No 27). Preferably, the promoter is a pyruvate:ferredoxin oxidoreductase promoter. In one particular embodiment, the promoter has the nucleic acid sequence of SEQ_ID NO. 5 or a functionally equivalent variant thereof. It will be appreciated by those of skill in the art that other promoters which can direct expression, preferably a high level of expression under appropriate fermentation conditions, would be effective as alternatives to the exemplified embodiments.

In one embodiment, the exogenous nucleic acid is an expression plasmid having the nucleotide sequence SEQ ID No. 17.

In one embodiment, the nucleic acids encoding the one or more enzymes, and optionally the promoter, are integrated into the genome of the microorganism. In other embodiment, the nucleic acids encoding the one or more enzymes are not integrated into the genome of the microorganism.

In one embodiment, the parental carboxydotrophic acetogenic microorganism is selected from the group consisting of Clostridium autoethanogenum, Clostridium ljungdahlii, Clostridium ragsdalei, Clostridium carboxidivorans, Clostridium drakei, Clostridium scatologenes, Butyribacterium limosum, Butyribacterium methylotrophicum, Acetobacterium woodii, Alkalibaculum bacchii, Blautia producta, Eubacterium limosum, Moorella thermoacetica, Moorella thermautotrophica, Oxobacter pfennigii, and Thermoanaerobacter kiuvi.

In one particular embodiment of the first or second aspects, the parental microorganism is selected from the group of carboxydotrophic Clostridia comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum.

In a one embodiment, the microorganism is selected from a cluster of carboxydotrophic Clostridia comprising the species C. autoethanogenum, C. ljungdahlii, and “C. ragsdalei” and related isolates. These include but are not limited to strains C. autoethanogenum JAI-1^{T} (DSM10061) (Abrini, Naveau, & Nyns, 1994), C. autoethanogenum LBS1560 (DSM19630) (WO/2009/064200), C. autoethanogenum LBS1561 (DSM23693), C. ljungdahlii PETC^{T} (DSM13528=ATCC 55383) (Tanner, Miller, & Yang, 1993), C. ljungdahlii ERI-2 (ATCC 55380) (U.S. Pat. No. 5,593,886), C. ljungdahlii C-01 (ATCC 55988) (U.S. Pat. No. 6,368,819), C. ljungdahlii O-52 (ATCC 55989) (U.S. Pat. No. 6,368,819), or “C. ragsdalei P11^{T}” (ATCC BAA-622) (WO 2008/028055), and related isolates such as “C. coskatii” (US patent 2011/0229947), “Clostridium sp. MT351” (Tyurin & Kiriukhin, 2012), “Clostridium sp. MT 653” (Berzin, Kiriukhin, & Tyurin, 2012a), “Clostridium sp. MT683” (Berzin & Tyurin, 2012), “Clostridium sp. MT962” (Berzin, Kiriukhin, & Tyurin, 2013) “Clostridium sp. MT1121” (Berzin, Kiriukhin, & Tyurin, 2012b), “Clostridium sp. MT1230” (Kiriukhin & Tyurin, 2013), or “Clostridium sp. MT1962” (Berzin, Tyurin, & Kiriukhin, 2013), and mutant strains thereof such as C. ljungdahlii OTA-1 (Tirado-Acevedo O. Production of Bioethanol from Synthesis Gas Using Clostridium ljungdahlii. PhD thesis, North Carolina State University, 2010) or “Clostridium sp. MT896” (Berzin, Kiriukhin, & Tyurin, 2012c).

These strains form a subcluster within the Clostridial rRNA cluster I (Collins et al., 1994), having at least 99% identity on 16S rRNA gene level, although being distinct species as determined by DNA-DNA reassociation and DNA fingerprinting experiments (WO 2008/028055, US patent 2011/0229947).

The strains of this cluster are defined by common characteristics, having both a similar genotype and phenotype, and they all share the same mode of energy conservation and fermentative metabolism. The strains of this cluster lack cytochromes and conserve energy via an Rnf complex.

All strains of this cluster have a genome size of around 4.2 MBp (Köpke et al., 2010) and a GC composition of around 32% mol (Abrini et al., 1994; Kopke et al., 2010; Tanner et al., 1993) (WO 2008/028055; US patent 2011/0229947), and conserved essential key gene operons encoding for enzymes of Wood-Ljungdahl pathway (Carbon monoxide dehydrogenase, Formyl-tetrahydrofolate synthetase, Methylene-tetrahydrofolate dehydrogenase, Formyl-tetrahydrofolate cyclohydrolase, Methylene-tetrahydrofolate reductase, and Carbon monoxide dehydrogenase/Acetyl-CoA synthase), hydrogenase, formate dehydrogenase, Rnf complex (rnfCDGEAB), pyruvate:ferredoxin oxidoreductase, aldehyde:ferredoxin oxidoreductase (Kopke et al., 2010, 2011). The organization and number of Wood-Ljungdahl pathway genes, responsible for gas uptake, has been found to be the same in all species, despite differences in nucleic and amino acid sequences (Köpke et al., 2011).

The strains all have a similar morphology and size (logarithmic growing cells are between 0.5-0.7×3-5 μm), are mesophilic (optimal growth temperature between 30-37° C.) and strictly anaerobe (Abrini et al., 1994; Tanner et al., 1993) (WO 2008/028055). Moreover, they all share the same major phylogenetic traits, such as same pH range (pH 4-7.5, with an optimal initial pH of 5.5-6), strong autotrophic growth on CO containing gases with similar growth rates, and a metabolic profile with ethanol and acetic acid as main fermentation end product, with small amounts of 2,3-butanediol and lactic acid formed under certain conditions (Abrini et al., 1994; Köpke et al., 2011; Tanner et al., 1993) However, the species differentiate in substrate utilization of various sugars (e.g. rhamnose, arabinose), acids (e.g. gluconate, citrate), amino acids (e.g. arginine, histidine), or other substrates (e.g. betaine, butanol). Some of the species were found to be auxotroph to certain vitamins (e.g. thiamine, biotin) while others were not. Reduction of carboxylic acids into their corresponding alcohols has been shown in a range of these organisms (Perez, Richter, Loftus, & Angenent, 2012).

The traits described are therefore not specific to one organism like C. autoethanogenum or C. ljungdahlii, but rather general traits for carboxydotrophic, ethanol-synthesizing Clostridia. Thus, the invention can be anticipated to work across these strains, although there may be differences in performance.

In certain embodiments, the parental carboxydotrophic actogenic microorganism is selected from the group comprising Clostridium autoethanogenum, Clostridium ljungdahlii, and Clostridium ragsdalei. In one embodiment, the group also comprises Clostridium coskatii. In one particular embodiment the parental microoragsnism is Clostridium ljungdahlii DSM13528(ATCC 55383). In another particular embodiment, the parental organism is Clostridium autoethanogenum DSM10061. In another particular embodiment, the parental microorganism is Clostridium autoethanogenum DSM23693, a derivate of Clostridium autoethanogenum DSM10061.

The DSM 23693 strain has been deposited with the Deutsche Sammlung für Mikroorganismen and Zellkulturen GmbH, Inhoffenstraβe 7 B, 38124Braunschweig, Germany (DSMZ) on 7 Jun. 2010.

In one embodiment, the parental microorganism lacks one or more genes encoding the enzymes referred to herein before.

The invention also provides nucleic acids and nucleic acid constructs of use in generating a recombinant microorganism of the invention.

The nucleic acids may encode one or more enzymes, which when expressed in a microorganism, result in the microorganism having an increased tolerance to ethanol. In one particular embodiment, the invention provides a nucleic acid encoding two or more enzymes, which when expressed in a carboxydotrophic acetogenic microorganism, results in the microorganism having an increased tolerance to ethanol. In one particular embodiment, the two or more enzymes are chosen from ClpB, ClpC, ClpP, DnaK, DnaJ, GreA, GroES, GroEL, GrpE, Hsp18, Hsp90, HtrA, Map, TufA, TufB, or YacI, or functionally equivalent variants thereof, in any order. Other embodiments include nucleic acids encoding at least 3, 4, 5 or 6 of ClpB, ClpC, ClpP, DnaK, DnaJ, GreA, GroES, GroEL, GrpE, Hsp18, Hsp90, HtrA, Map, TufA, TufB, or YacI, or a functionally equivalent variant of any one or more thereof, in any order.

Exemplary amino acid sequences and nucleic acid sequence encoding each of the above enzymes is provided in GenBank as herein before described. However, skilled persons will readily appreciate alternative nucleic acids sequences encoding the enzymes or functionally equivalent variants thereof, having regard to the information contained herein, in GenBank and other databases, and the genetic code.

In one embodiment, the nucleic acid encodes both GroES and GroEL. In one particular embodiment, the nucleic acid comprises SEQ_ID No 3 and 4, or functionally equivalent variants thereof, in any order. In one embodiment, the nucleic acid comprises SEQ ID_NO. 12, or a functionally equivalent variant thereof.

In one embodiment, the nucleic acid encodes GrpE, DnaK, DnaJ. In one particular embodiment, the nucleic acid comprises SEQ_ID No 35 and 37 and 39, or functionally equivalent variants thereof, in any order. In one embodiment, the nucleic acid comprises SEQ ID_{—}41, or a functionally equivalent variant thereof.

In one embodiment, the nucleic acids of the invention will further comprise a promoter. Preferably, the promoter is as herein before described, and in a particular embodiment a pyruvate:ferredoxin oxidoreductase promoter. In one particular embodiment, the promoter has the nucleic acid sequence of SEQ_ID NO. 5 or a functionally equivalent variant thereof.

The nucleic acids of the invention may remain extra-chromosomal upon transformation of a parental microorganism or may be adapted for integration into the genome of the microorganism. Accordingly, nucleic acids of the invention may include additional nucleotide sequences adapted to assist integration (for example, a region which allows for homologous recombination and targeted integration into the host genome) or stable expression and replication of an extrachromosomal construct (for example, origin of replication, promoter and other regulatory sequences).

In one embodiment, the nucleic acid is a nucleic acid construct or vector. In one particular embodiment, the nucleic acid construct or vector is an expression construct or vector, however other constructs and vectors, such as those used for cloning are encompassed by the invention. In one particular embodiment, the expression construct or vector is a plasmid.

In one particular embodiment, the invention provides an expression construct or vector comprising a nucleic acid sequence encoding at least one enzyme, preferable two or more enzymes, which when expressed in a carboxydotrophic acetogenic microorganism, results in the microorganism having an increased tolerance to ethanol. Preferably, the enzymes are as referred to herein before.

In one embodiment, the expression construct/vector comprises nucleic acid sequences encoding each of GroES (SEQ ID No. 1) and GroEL (SEQ_ID NO. 2). In one particular embodiment, the expression construct/vector comprises the nucleic acid sequences SEQ_ID NO. 3 and 4 or a functionally equivalent variant thereof, in any order. In one embodiment, the expression construct/vector comprises SEQ ID_NO. 12, or a functionally equivalent variant thereof.

In one embodiment, the expression construct/vector comprises nucleic acid sequences encoding each of GrpE (SEQ ID No. 35), DnaJ (SEQ ID No. 37) and DnaK (SEQ_ID NO. 39). In one particular embodiment, the expression construct/vector comprises the nucleic acid sequences SEQ_ID NO. 35 and 37 and 41 or a functionally equivalent variant thereof, in any order. In one embodiment, the expression construct/vector comprises SEQ ID_NO. 41, or a functionally equivalent variant thereof.

Preferably the expression construct/vector will further comprise a promoter, as herein before described. In one embodiment, the promoter allows for constitutive expression of the genes under its control. However, inducible promoters may also be employed. It will be appreciated by those of skill in the art that other promoters which can direct expression, preferably a high level of expression under appropriate fermentation conditions, would be effective as alternatives to the presently preferred embodiments.

It will be appreciated that an expression construct/vector of the present invention may contain any number of regulatory elements in addition to the promoter as well as additional genes suitable for expression of further proteins if desired. In one embodiment the expression construct/vector includes one promoter. In another embodiment, the expression construct/vector includes two or more promoters. In one particular embodiment, the expression construct/vector includes one promoter for each gene to be expressed. In one embodiment, the expression construct/vector includes one or more ribosomal binding sites, preferably a ribosomal binding site for each gene to be expressed.

It will be appreciated by those of skill in the art that the nucleic acid sequences and construct/vector sequences described herein may contain standard linker nucleotides such as those required for ribosome binding sites and/or restriction sites. Such linker sequences should not be interpreted as being required and do not provide a limitation on the sequences defined.

In one particular embodiment of the invention, the expression construct/vector is an expression plasmid comprising the nucleotide sequence SEQ ID No. 17.

The invention also provides nucleic acids which are capable of hybridising to at least a portion of a nucleic acid herein described, a nucleic acid complementary to any one thereof, or a functionally equivalent variant of any one thereof. Such nucleic acids will preferably hybridise to such nucleic acids, a nucleic acid complementary to any one thereof, or a functionally equivalent variant of any one thereof, under stringent hybridisation conditions. “Stringent hybridisation conditions” means that the nucleic acid is capable of hybridising to a target template under standard hybridisation conditions such as those described in Sambrook et al, Molecular Cloning: A Laboratory Manual (1989), Cold Spring Harbor Laboratory Press, New York, USA. It will be appreciated that the minimal size of such nucleic acids is a size which is capable of forming a stable hybrid between a given nucleic acid and the complementary sequence to which it is designed to hybridise. Accordingly, the size is dependent on the nucleic acid composition and percent homology between the nucleic acid and its complementary sequence, as well as the hybridisation conditions which are utilised (for example, temperature and salt concentrations). In one embodiment, the nucleic acid is at least 10 nucleotides in length, at least 15 nucleotides in length, at least, 20 nucleotides in length, at least 25 nucleotides in length, or at least 30 nucleotides in length.

In one embodiment the invention provides a nucleic acid consisting of the sequence of any one of SEQ ID NO.s 6, 7, 8, 9, 10, 11, 29, 30, 31, 32, 33, 34, 42, 43, 44, 45, 49, 50, 51, 54, and 55.

Nucleic acids and nucleic acid constructs, including the expression construct/vector of the invention may be constructed using any number of techniques standard in the art. For example, chemical synthesis or recombinant techniques may be used. Such techniques are described, for example, in Sambrook et al (Molecular Cloning: A laboratory manual, Cold Spring Harbor Laboratory Press, Cold Spring Harbor, N.Y., 1989). Further exemplary techniques are described in the Examples section herein after. Essentially, the individual genes and regulatory elements will be operably linked to one another such that the genes can be expressed to form the desired proteins. Suitable vectors for use in the invention will be appreciated by those of ordinary skill in the art. However, by way of example, the following vectors may be suitable: pMTL80000 shuttle vectors, pIMP1, pJIR750 and the plasmids exemplified in the Examples section herein after.

It should be appreciated that nucleic acids of the invention may be in any appropriate form, including RNA, DNA, or cDNA, including double-stranded and single-stranded nucleic acids.

The invention also provides host organisms, particularly microorganisms, and including viruses, bacteria, and yeast, comprising any one or more of the nucleic acids described herein.

The one or more exogenous nucleic acids may be delivered to a parental carboxydotrophic acetogenic microorganism as naked nucleic acids or may be formulated with one or more agents to facilitate the tranformation process (for example, liposome-conjugated nucleic acid, an organism in which the nucleic acid is contained). The one or more nucleic acids may be DNA, RNA, or combinations thereof, as is appropriate.

The recombinant carboxydotrophic acetogenic microorganisms of the invention may be prepared from a parental carboxydotrophic acetogenic microorganism and one or more exogenous nucleic acids using any number of techniques known in the art for producing recombinant microorganisms. By way of example only, transformation (including transduction or transfection) may be achieved by electroporation, electrofusion, ultrasonication, polyethylene glycol-mediated transformation, conjugation, or chemical and natural competence. Suitable transformation techniques are described for example in Sambrook J, Fritsch E F, Maniatis T: Molecular Cloning: A laboratory Manual, Cold Spring Harbour Labrotary Press, Cold Spring Harbour, 1989.

Electroporation has been described for several carboxydotrophic acetogens as C. ljungdahlii (Köpke et al. 2010, Poc. Nat. Acad. Sci. U.S.A. 107: 13087-92; Leang et al., 2012, Appl. Environ. Microbiol.; PCT/NZ2011/000203; WO2012/053905), C. autoethanogenum (PCT/NZ2011/000203; WO2012/053905), Acetobacterium woodii (Straetz et al., 1994, Appl. Environ. Microbiol. 60:1033-37) or Moorella thermoacetica (Kita et al., 2012) and is a standard method used in many Clostridia such as C. acetobutylicum (Mermelstein et al., 1992, Biotechnology, 10, 190-195), C. cellulolyticum (Jennert et al., 2000, Microbiology, 146: 3071-3080) or C. thermocellum (Tyurin et al., 2004, Appl. Environ. Microbiol. 70: 883-890).

Electrofusion has been described for acetogenic Clostridium sp. MT351 (Tyurin and Kiriukhin, 2012, J Biotech: 1-12).

Prophage induction has been described for carboxydotrophic acetogen as well in case of C. scatologenes (Prasanna Tamarapu Parthasarathy, 2010, Development of a Genetic Modification System in Clostridium scatologenes ATCC 25775 for Generation of Mutants, Masters Project Western Kentucky University).

Conjugation has been described as method of choice for acetogen Clostridium difficile (Herbert et al., 2003, FEMS Microbiol. Lett. 229: 103-110) and many other Clostridia including C. acetobuylicum (Williams et al., 1990, J. Gen. Microbiol. 136: 819-826).

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant carboxydotrophic acetogenic microorganism of the invention is produced by a method comprises the following steps:
a. introduction into a shuttle microorganism of (i) of an expression construct/vector as described herein and (ii) a methylation construct/vector comprising a methyltransferase gene;
b. expression of the methyltransferase gene;
c. isolation of one or more constructs/vectors from the shuttle microorganism; and,
d. introduction of the one or more construct/vector into a destination microorganism.

In one embodiment, the methyltransferase gene of step (b) is expressed consitutively. In another embodiment, expression of the methyltransferase gene of step (b) is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtillis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector in induced. Induction may be by any suitable promoter system although in one particular embodiment of the invention, the methylation construct/vector comprises an inducible lac promoter (preferably encoded by SEQ_ID NO 19) and is induced by addition of lactose or an analogue thereof, more preferably isopropyl-β-D-thio-galactoside (IPTG). Other suitable promoters include the ara, tet, or T7 system. In a further embodiment of the invention, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant carboxydotrophic acetogenic microorganisms of the invention.

In one particular embodiment, the expression construct/vector and/or the methylation construct/vector are plasmids.

Skilled person will appreciate a number of suitable methyltransferases of use in producing the microorganisms of the invention. However, by way of example the Bacillus subtilis phage ΦT1 methyltransferase and the methyltransferase described in the Examples herein after may be used. Nucleic acids encoding suitable methyltransferases will be readily appreciated having regard to the sequence of the desired methyltransferase and the genetic code. In one embodiment, the nucleic acid encoding a methyltransferase is described in the Examples herein after (for example the nucleic acid of SEQ_ID NO. 28.

Any number of constructs/vectors adapted to allow expression of a methyltransferase gene may be used to generate the methylation construct/vector. However, by way of example, the plasmid described in the Examples section hereinafter may be used. In one particular embodiment, the plasmid has the sequence of SEQ_ID NO. 19.

The invention provides a method for the production ethanol or one or more other products by microbial fermentation comprising fermenting a substrate comprising CO using a recombinant microorganism of the invention. The methods of the invention may be used to reduce the total atmospheric carbon emissions from an industrial process.

Preferably, the fermentation comprises the steps of anaerobically fermenting a substrate in a bioreactor to produce ethanol, or ethanol and one or more other products using a recombinant microorganism of the invention.

In one embodiment the method comprises the steps of:
(a) providing a substrate comprising CO to a bioreactor containing a culture of one or more recombinant carboxydotrophic acetogenic microorganism of the first aspect of the invention; and
(b) anaerobically fermenting the culture in the bioreactor to produce one or more products including ethanol.

In one embodiment the method comprises the steps of:
(a) capturing CO-containing gas produced as a result of the industrial process, before the gas is released into the atmosphere;
(b) the anaerobic fermentation of the CO-containing gas to produce one or more products including ethanol by a culture containing one or more recombinant carboxydotrophic acetogenic microorganism of the first aspect of the invention.

In one embodiment, the recombinant carboxydotrophic microorganism is tolerant of ethanol concentration in the fermentation broth of at least about 5.5% by weight. In another embodiment, the recombinant carboxydotrophic microorganism is tolerant of ethanol concentration in the fermentation broth of at least approximately 6% by weight. In a further embodiment the recombinant carboxydotrophic microorganism is tolerant of ethanol concentration in the fermentation broth of from about 3 to about 15% by weight. In another embodiment the recombinant carboxydotrophic microorganism is tolerant of ethanol concentration in the fermentation broth of from about 5.5 to about 15% by weight or from about 6% to about 15% by weight or from about 5.5% to about 10% by weight.

In an embodiment of the invention, the gaseous substrate fermented by the microorganism is a gaseous substrate comprising CO. The gaseous substrate may be a CO-containing waste gas obtained as a by-product of an industrial process, or from some other source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. The CO may be a component of syngas (gas comprising carbon monoxide and hydrogen). The CO produced from industrial processes is normally flared off to produce CO₂ and therefore the invention has particular utility in reducing CO₂ greenhouse gas emissions and producing butanol for use as a biofuel. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

It will be appreciated that for growth of the bacteria and CO-to-ethanol (and/or other product(s)) to occur, in addition to the CO-containing substrate gas, a suitable liquid nutrient medium will need to be fed to the bioreactor. The substrate and media may be fed to the bioreactor in a continuous, batch or batch fed fashion. A nutrient medium will contain vitamins and minerals sufficient to permit growth of the micro-organism used. Anaerobic media suitable for fermentation to produce ethanol (and optionally one or more other products) using CO are known in the art. For example, suitable media are described in Biebel (Journal of Industrial Microbiology & Biotechnology (2001) 27, 18-26). The substrate and media may be fed to the bioreactor in a continuous, batch or batch fed fashion. In one embodiment of the invention the media is as described in the Examples section herein after.

The fermentation should desirably be carried out under appropriate conditions for the CO-to-ethanol (and/or other product(s)) fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

In addition, it is often desirable to increase the CO concentration of a substrate stream (or CO partial pressure in a gaseous substrate) and thus increase the efficiency of fermentation reactions where CO is a substrate. Operating at increased pressures allows a significant increase in the rate of CO transfer from the gas phase to the liquid phase where it can be taken up by the micro-organism as a carbon source for the production of ethanol (and/or other product(s)). This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the invention used. However, in general, it is preferred that the fermentation be performed at pressure higher than ambient pressure. Also, since a given CO-to-ethanol (and/or other product(s)) conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefit of conducting a gas-to-ethanol fermentation at elevated pressures has been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the ethanol product is consumed by the culture.

The composition of gas streams used to feed a fermentation reaction can have a significant impact on the efficiency and/or costs of that reaction. For example, O₂ may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a bacterium of the invention is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

Ethanol, or a mixed alcohol stream containing ethanol and one or more other alcohols, or a mixed product stream comprising ethanol and/or one or more other products, may be recovered from the fermentation broth by methods known in the art, such as fractional distillation or evaporation, pervaporation, and extractive fermentation, including for example, liquid-liquid extraction. By-products such as acids including acetate may also be recovered from the fermentation broth using methods known in the art. For example, an adsorption system involving an activated charcoal filter or electrodialysis may be used. Alternatively, continuous gas stripping may also be used.

In certain preferred embodiments of the invention, ethanol and/or one or more other products are recovered from the fermentation broth by continuously removing a portion of the broth from the bioreactor, separating microbial cells from the broth (conveniently by filtration), and recovering one or more products from the broth. Alcohols may conveniently be recovered for example by distillation, and acids may be recovered for example by adsorption on activated charcoal. The separated microbial cells are preferably returned to the fermentation bioreactor. The cell free permeate remaining after any alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### EXAMPLES

The invention will now be described in more detail with reference to the following non-limiting examples.

### Microorganism

The following work was conducted using C. ljungdahlii DSM13583, C. autoethanogenum DSM10061, Clostridium autoethanogenum deposited with DSMZ (The German Collection of Microorganisms and Cell Cultures), Inhoffenstraβe 7 B, 38124 Braunschweig, GERMANY.

### Example 1

### Ethanol Tolerance of Clostridium ljungdahlii DSM13528 and Clostridium autoethanogenum DSM10061 and DSM23693 Strains

The ethanol tolerance of three acetogenic strains Clostridium ljungdahlii DSM13583, Clostridium autoethanogenum DSM10061, and C. autoethanogenum DSM23693 was tested in serum bottles. It was found that the ethanol tolerance of the strains varied, with C. autoethanogenum DSM23693 being the most tolerant strains, followed by Clostridium ljungdahlii DSM13528 and Clostridium autoethanogenum DSM10061. However, none of the strains was able to grow in presence of 50 g/L ethanol in serum bottle studies.

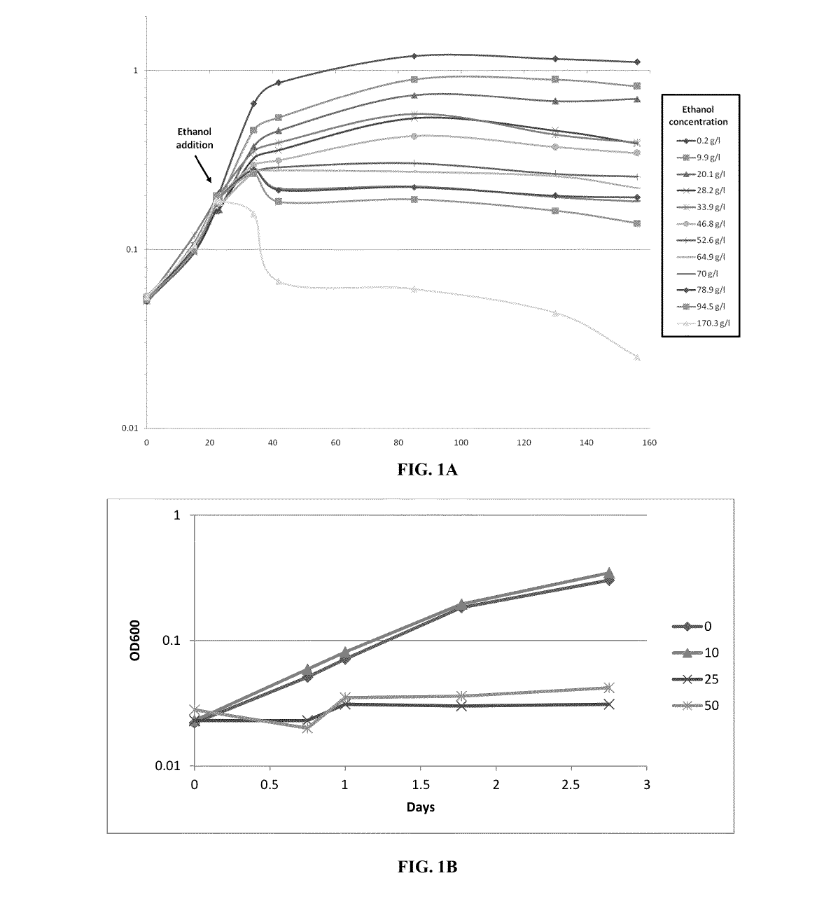

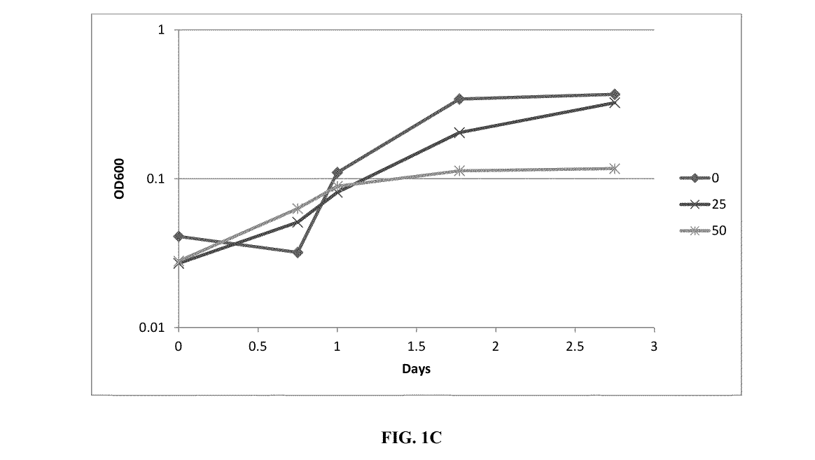

For Clostridium autoethanogenum DSM23693, growth was found to be inhibited at concentrations between 10-20 g/l ethanol, while growth completely ceased after addition of >50 g/l or >5% (w/v) ethanol (FIG. 1a).

For Clostridium autoethanogenum DSM10061, growth was found that growth already ceased after addition of >25 g/L or >2.5% (w/v) ethanol (FIG. 1b).

For Clostridium ljungdahlii DSM13583, growth ceased after addition of 25-50 g/L or 2.5-5% (w/v) ethanol (FIG. 1c).

Ethanol was added in various concentrations to an active growing culture at 37° C. in PETC medium (Table 1) with 30 psi steel mill gas as substrate. The media was prepared by using standard anaerobic techniques (Hungate R E. A roll tube method for cultivation of strict anaerobes, In Norris J R and Ribbons D W (eds.), Methods in Microbiology, vol. 3B. Academic Press, NY, 1969: 117-132; Breznak J A and Costilow R N, Physicochemical factors in growth, In Gerhardt P (ed.), Methods for general and molecular bacteriology. American Society for Microbiology, Washington, 1994: 137-154). Ethanol concentrations were confirmed by HPLC analysis using an Agilent 1100 Series HPLC system equipped with a RID (Refractive Index Detector) operated at 35° C. and an Alltech IOA-2000 Organic acid column (150×6.5 mm, particle size 5 μm) kept at 60° C. Slightly acidified water was used (0.005 M H₂SO₄) as mobile phase with a flow rate of 0.7 ml/min. To remove proteins and other cell residues, 400 μl samples were mixed with 100 μl of a 2% (w/v) 5-Sulfosalicylic acid and centrifuged at 14,000×g for 3 min to separate precipitated residues. 10 μl of the supernatant were then injected into the HPLC for analyses.

TABLE 1
PETC medium

| Media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution (see below) | 10 | ml |
| Wolfe's vitamin solution (see below) | 10 | ml |
| Yeast Extract | 1 | g |
| Resazurin (2 g/L stock) | 0.5 | ml |
| NaHCO₃ | 2 | g |
| Reducing agent | 0.006-0.008% | (v/v) |
| Wolfe's vitamin solution | Per L of Stock | |
| Biotin | 2 | mg |
| Folic acid | 2 | mg |
| Pyridoxine hydrochloride | 10 | mg |
| Thiamine•HCl | 5 | mg |
| Riboflavin | 5 | mg |
| Nicotinic acid | 5 | mg |
| Calcium D-(+)-pantothenate | 5 | mg |
| Vitamin B₁₂ | 0.1 | mg |
| p-Aminobenzoic acid | 5 | mg |
| Thioctic acid | 5 | mg |
| Trace metal solution | Per L of stock | |
| Nitrilotriacetic Acid | 2 | g |
| MnSO₄•H₂O | 1 | g |
| Fe (SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |
| Reducing agent stock | Per 100 mL of stock | |
| NaOH | 0.9 | g |
| Cystein•HCl | 4 | g |
| Na₂S | 4 | g |

### Example 2

### Genetic Modification of Clostridium autoethanogenum DSM23693, C. autoethanogenum DSM10061 And C. ljungdahlii DSM13528 with Chaperons GroES and GroEL for Improved Ethanol Tolerance and Production

In example 1, it has been shown that ethanol concentrations in range of 25-50 g/l or 2.5-5% (w/v) have been shown to inhibit the growth of Clostridium autoethanogenum DSM23693, C. autoethanogenum DSM10061 and C. ljungdahlii DSM13528 completely (FIG. 1) and thus form a physical limit for the production of ethanol. When Heat shock protein/chaperonin GroES (SEQ_ID NO. 1) and GroEL (SEQ_ID NO. 2) were overproduced in Clostridium autoethanogenum DSM23693, C. autoethanogenum DSM10061 and heterologously expressed in C. ljungdahlii DSM13583, it was surprisingly found that overproduction of these chaperons GroES and GroEL conferred higher tolerance of all three acetogenic carboxydotrophic strains to ethanol while allowing faster growth and at the same time enhancing ethanol production during growth on CO. In addition, it was surprisingly found that the promoter used for chaperon overexpression has an important role in ethanol tolerance which appears to be hard to predict.

Promoter Sequences for Gene Overexpression in C. autoethanogenum and Heterologous Expression in C. ljungdahlii:

For overexpression of genes groES (SEQ_ID NO. 3) and groEL (SEQ_ID NO. 4), a strong native pyruvate:ferredoxin oxidoreductase promoter was used. This gene was found to be constitutively expressed at a high level (FIG. 2). In addition, two other strong promoters were used, the phosphotransacetylase/acetate kinase operon and the Wood-Ljungdahl cluster promoter to evaluate the effect of the promoter sequence on enhancing ethanol tolerance by chaperon overproduction.

### Amplification of Genes and Promoter Sequences:

Standard Recombinant DNA and molecular cloning techniques were used in this invention (Sambrook J, Fritsch E F, Maniatis T: Molecular Cloning: A laboratory Manual, Cold Spring Harbour Labrotary Press, Cold Spring Harbour, 1989; Ausubel F M, Brent R, Kingston R E, Moore D D, Seidman J G, Smith J A, Struhl K: Current protocols in molecular biology. John Wiley & Sons, Ltd., Hoboken, 1987). DNA sequences of groES and groEL genes and pyruvate:ferredoxin oxidoreductase (P_{pfor}), the phosphotransacetylase/acetate kinase operon and the Wood-Ljungdahl cluster promoter were sequenced from C. autoethanogenum (Table 2).

TABLE 2
Gene sequences

| | | SEQ |
|---|---|---|
| Gene/Promoter | Description | ID NO. |
| groES | Clostridium autoethanogenum | 3 |
| groEL | Clostridium autoethanogenum | 4 |
| Pyruvate: ferredoxin | Clostridium autoethanogenum | 5 |
| oxidoreductase promoter | | |
| (P_{PFOR}) | | |
| phosphotransacetylase/acetate | Clostridium autoethanogenum | 24 |
| kinase operon promoter | | |
| (P_{Pta-Ack}) | | |
| Wood-Ljungdahl cluster | Clostridium autoethanogenum | 25 |
| promoter (P_{WL}) | | |

Genomic DNA from Clostridium autoethanogenum DSM 10061 and DSM23693 was isolated using a modified method by Bertram and Dune (1989), 1989 (Conjugal transfer and expression of streptococcal transposons in Clostridium acetobutylicum. Arch Microbiol 151: 551-557). A 100-ml overnight culture was harvested (6,000×g, 15 min, 4° C.), washed with potassium phosphate buffer (10 mM, pH 7.5) and suspended in 1.9 ml STE buffer (50 mM Tris-HCl, 1 mM EDTA, 200 mM sucrose; pH 8.0). 300 μl lysozyme (˜100,000 U) was added and the mixture was incubated at 37° C. for 30 min, followed by addition of 280 μl of a 10% (w/v) SDS solution and another incubation for 10 min. RNA was digested at room temperature by addition of 240 μl of an EDTA solution (0.5 M, pH 8), 20 μl Tris-HCl (1 M, pH 7.5), and 10 μl RNase A (Fermentas Life Sciences). Then, 100 μl Proteinase K (0.5 U) was added and proteolysis took place for 1-3 h at 37° C. Finally, 600 μl of sodium perchlorate (5 M) was added, followed by a phenol-chloroform extraction and an isopropanol precipitation. DNA quantity and quality was inspected spectrophotometrically.

All sequences were amplified from isolated genomic DNA by PCR with oligonucleotides given in Table 3 using iProof High Fidelity DNA Polymerase (Bio-Rad Labratories) and the following program: initial denaturation at 98° C. for 30 seconds, followed by 32 cycles of denaturation (98° C. for 10 seconds), annealing (50-62° C. for 30-120 seconds) and elongation (72° C. for 30-90 seconds), before a final extension step (72° C. for 10 minutes).

TABLE 3
Oligonucleotides for cloning

| | | | SEQ_ |
|---|---|---|---|
| | Oligonucleotide | | ID |
| Target | Name | DNA Sequence (5′ to 3′) | NO. |
| groESL operon | SOE-GroESL-a- | GGGTTCATATGAAAATTAGACC | 6 |
| | NdeI | ACTTGG | |
| groESL operon | SOE-GroESL-b | TCCCATGTTTTCATAAGGATCTT | 7 |
| | | CTAATTC | |
| groESL operon | SOE-GroESL-c | ATTAGAAGATCCTTATGAAAAC | 8 |
| | | ATGGGAGC | |
| groESL operon | SOE-GroESL-d- | CTTAGAATTCCTTTTGAATTAGT | 9 |
| | EcoRI | ACATTCC | |
| Pyruvate: | Ppfor-NotI-F | AAGCGGCCGCAAAATAGTTGAT | 10 |
| ferredox in | | AATAATGC | |
| oxidoreductase | | | |
| promoter (P_{pfor}) | | | |
| Pyruvate: | Ppfor-NdeI-R | TACGCATATGAATTCCTCTCCTT | 11 |
| ferredox in | | TTCAAGC | |
| oxidoreductase | | | |
| promoter (P_{pfor}) | | | |
| Promoter of | Pwl-NotI-F | AAGCGGCCGCAGATAGTCATAATAGTT | 44 |
| Wood-Ljungdahl | | CC | |
| cluster of C. | | | |
| autoethanogenum | | | |
| Promoter of | Pwl-NdeI-R | TTCCATATGAATAATTCCCTCCTTAAAGC | 45 |
| Wood-Ljungdahl | | | |
| cluster of C. | | | |
| autoethanogenum | | | |
| Promoter | Ppta-ack-NotI-F | GAGCGGCCGCAATATGATATTTATGTCC | 60 |
| of phospho- | | | |
| transacetylase- | | | |
| acetate | | | |
| operon of C. | | | |
| autoethanogenum | | | |
| Promoter | Ppta-ack-NdeI-R | TTCCATATGTTTCATGTTCATTTCCTCC | 61 |
| of phospho- | | | |
| transacetylase- | | | |
| acetate | | | |
| operon of C. | | | |
| autoethanogenum | | | |

Genes groES and groEL were found to form a common operon on the genome of Clostridium autoethanogenum. The whole operon was amplified by SOE (splicing by overlap extension) PCR (Heckman K L, Pease L R: Gene Splicing and Mutagenesis by PCR-Driven Overlap Extension. Nature Protocols 2007, 2: 924-932; Vallejo A N, Pogulis R J, Pease L R: In Vitro Synthesis of Novel Genes: Mutagenesis and Recombination by PCR. Genome Research 1994, 4: S123-S130) in order to mutate an obstructing NdeI restriction site (CTTATG for CTGATG) within the groEL gene while retaining the same amino acid sequence (SEQ_ID NO. 12).

Initial PCRs using internal primer pairs “SOE-GroESL-a-NdeI” (SEQ_ID NO. 6) plus “SOE-GroESL-b” (SEQ_ID NO. 7) and “SOE-GroESL-c” (SEQ_ID NO. 8) plus “SOE-GroESL-d-EcoRI” (SEQ_ID NO. 9) generated overlapping fragments with complementary 3′ ends and a mutated NdeI site. These intermediate segments were then used as template for a second PCR using flanking oligonucleotides “SOE-GroESL-a-NdeI” (SEQ_ID NO. 6) and “SOE-GroESL-d-EcoRI” (SEQ_ID NO. 9) to create the full length product of the groESL operon without internal NdeI site (SEQ_ID NO. 12).

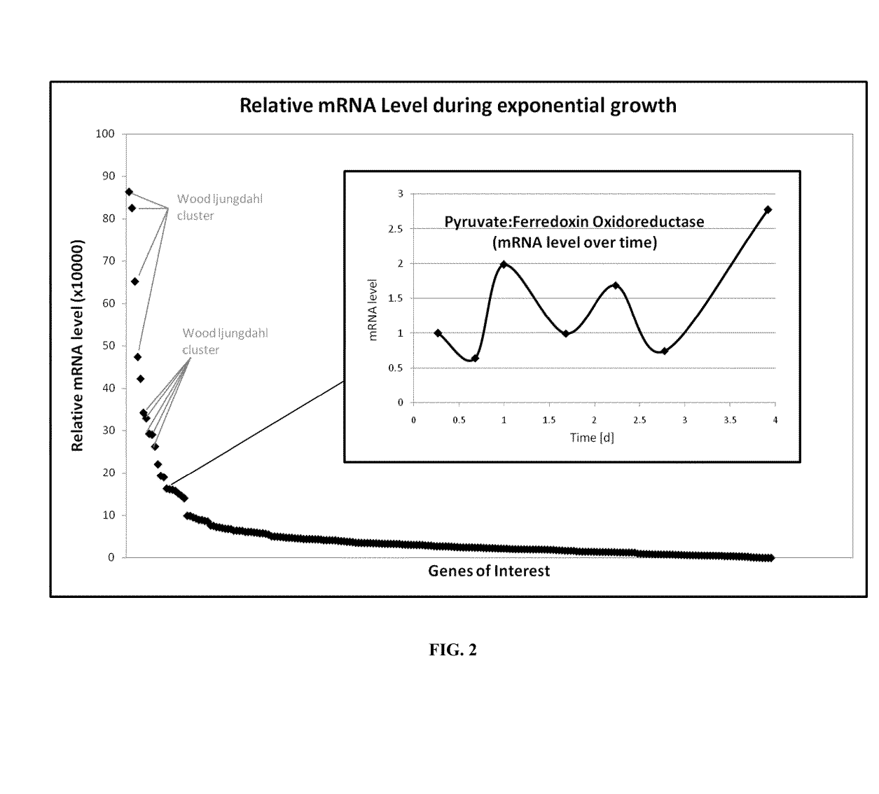

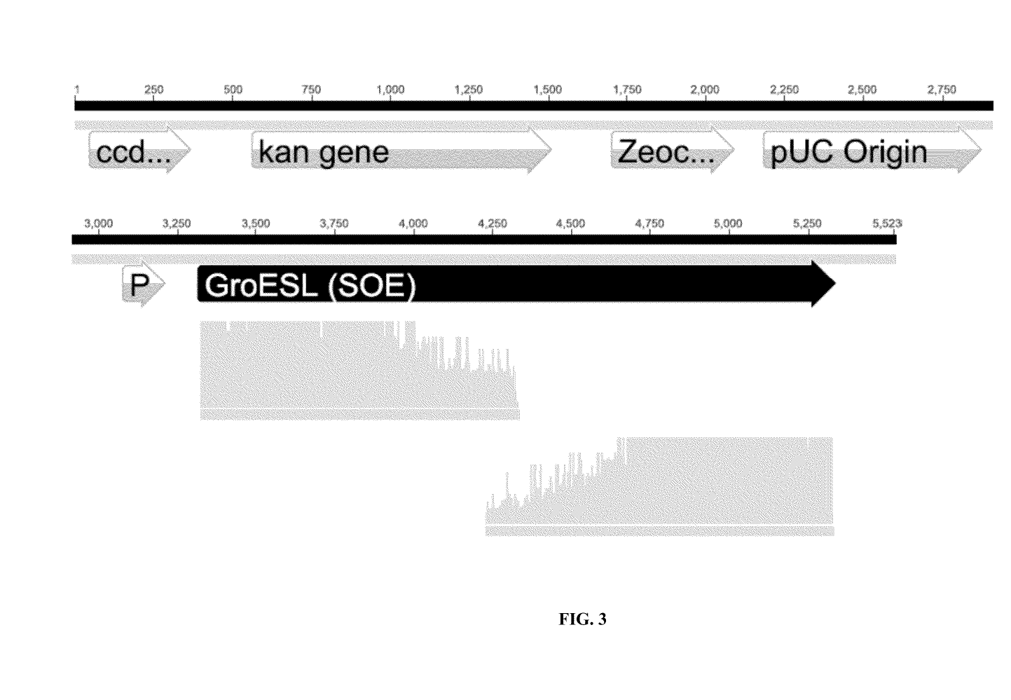

The PCR product was then cloned into vector pCR-Blunt II-TOPO, forming plasmid pCR-Blunt-GroESL, using Zero Blunt TOPO PCR cloning kit (Invitrogen) and E. coli strain DH5α-T1^{R} (Invitrogen). DNA sequencing using oligonucleotides M13 Forward (−20) (SEQ_ID NO. 13) and M13 Reverse (SEQ_ID NO. 14) showed that the groESL insert was free of mutation and the internal NdeI site was successfully mutated (FIG. 3).

Construction of a groESL Expression Plasmid:

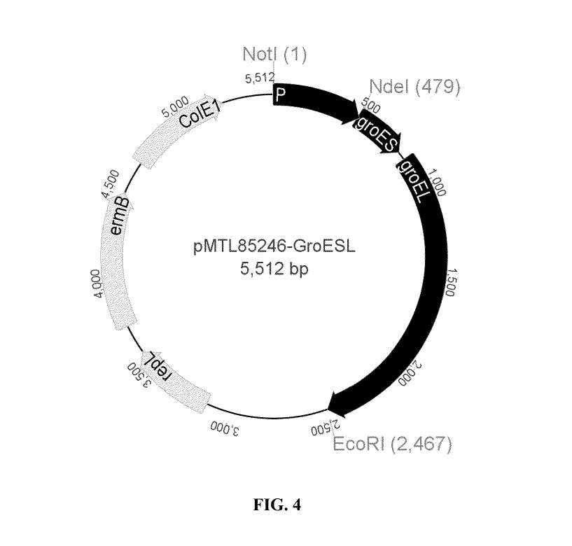

Construction of an expression plasmid was performed in E. coli DH5α-T1^{R} (Invitrogen). In a first step, the amplified pyruvate:ferredoxin oxidoreductase promoter region was cloned into the E. coli—Clostridium shuttle vector pMTL85141 (SEQ_ID NO. 15; FJ797651.1; Nigel Minton, University of Nottingham; Heap et al., 2009) using NotI and NdeI restriction sites, generating plasmid pMTL85146. As a second step, the antibiotic resistance marker was exchanged from catP to ermB (released from vector pMTL82254 (SEQ_ID NO. 16; FJ797646.1; Nigel Minton, University of Nottingham; Heap et al., 2009)) using restriction enzymes PmeI and FseI. The resulting plasmid pMTL85246 was then digested with NdeI and EcoRI and ligated with the groESL insert, which was released from plasmid pCR-Blunt-GroESL with NdeI and EcoRI, generating plasmid pMTL85246-GroESL (FIG. 4; SEQ_ID NO. 17). A different expression plasmid (with another antibiotic resistance marker) was created by releasing the groESL insert from pMTL85246-groESL with NdeI and SacI, and then clone into pMTL83156, generating pMTL83156-groESL (Error! Reference source not found; SEQ_ID 13).

DNA sequencing using oligonucleotides M13 Forward (−20) (SEQ_ID NO. 13) and M13 Reverse (SEQ_ID NO. 14) confirmed successful cloning (FIG. 5).

### Construction of a Control Plasmid:

A control plasmid that confers the same antibiotic resistance as the chaperone overexpressing plasmids was designed as control for alcohol tolerance assays. Plasmid pMTL83157 (Error! Reference source not found; SEQ_ID 48) was constructed by first amplifying the promoter region of the Wood-Ljungdahl cluster using primer pairs Pwl-NotI-F and Pwl-NdeI-R (Table). The PCR product and plasmid pMTL83151 were digested with restriction enzymes NotI and NdeI, followed by ligation to generate plasmid pMTL83157.

### Methylation of DNA:

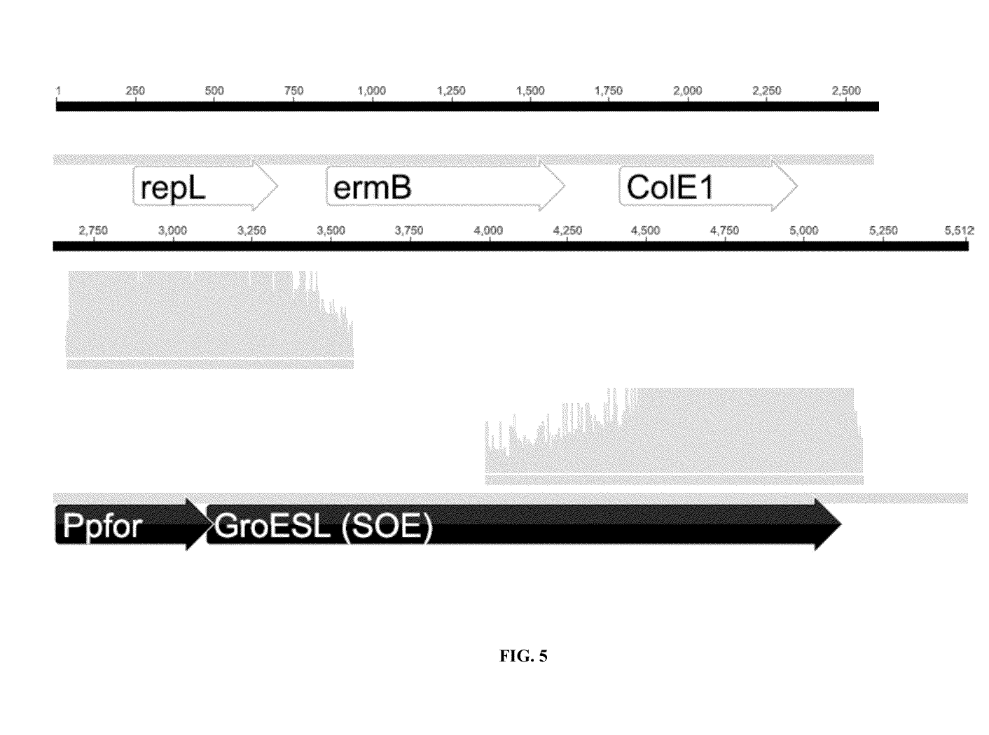

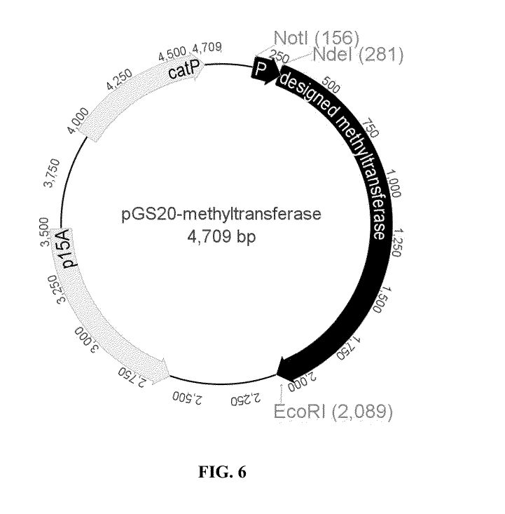

Transformation in Clostridium autoethanogenum DSM10061 and DSM23693 and C. ljungdahlii DSM13528 is faciliated by using methylated DNA, due to the presence of various restriction systems. Methylation of plasmid DNA was created in vivo in the restriction negative E. coli strain XL1-blue MRF' with a plasmid encoded Type II methyltransferase (SEQ_ID NO. 18). The methyltransferase was design according the sequences of a methyltransferase of C. autoethanogenum, C. ragsdalei and C. ljungdahlii and then chemically synthesized and cloned into plasmid pGS20 (ATG:biosynthetics GmbH, Merzhausen, Germany) under control of an inducible lac promoter (FIG. 6; SEQ_ID NO. 19). Expression and methylation plasmid were co-transformed in E. coli and methylation induced by addition of 1 mM IPTG. Isolated plasmid mix (QIAGEN Plasmid Midi Kit; QIAGEN), was used for transformation, but only the expression plasmid pMTL85246-GroESL has a Gram-(+) replication origin.

Transformation of groESL Expression Plasmid in C. autoethanogenum DSM23693, C. autoethanogenum DSM10061 and C. ljungdahlii DSM13583:

Competent cells of C. autoethanogenum DSM23693, C. autoethanogenum DSM10061 and C. ljungdahlii DSM13528 were made from a 50 ml culture grown in MES media (Table 4) and in presence of 40 mM threonine. At an OD₆₀₀ₙₘ of 0.4 (early to mid exponential growth phase), the cells were transferred into an anaerobic chamber and harvested at 4,700×g and 4° C. The culture was twice washed with ice-cold electroporation buffer (270 mM sucrose, 1 mM MgCl₂, 7 mM sodium phosphate, pH 7.4) and finally suspended in a volume of 500 μA fresh electroporation buffer. This mixture was transferred into a pre-cooled electroporation cuvette with a 0.4 cm electrode gap containing ˜1 μg of the methylated plasmid mix and 1 μl Type I restriction inhibitor (EPICENTRE). After a pulse (2.5 kV, 600Ω, and 25 μF; time constant 4.5-4.7 ms) was applied using a Gene pulser Xcell electroporation system (Bio-Rad) the cells were regenerated for 8 hours in MES media and then plated on PETC media (Table 1) plates (1.2% Bacto™ Agar (Becton Dickinson) containing 4 μg/ml clarithromycin respectively 7.5 μg/mL thiamphenicol (depending on the antibiotic marker cassette used) and 30 psi steel mill gas in the headspace. After 4-5 days, around 100 colonies were visible, which were used to inoculate selective liquid PETC media.

TABLE 4
MES media

| Media component | Concentration per 1.0 L of media | |
|---|---|---|
| NH₄Cl | 1 | g |
| KCl | 0.1 | g |
| MgSO₄•7H₂O | 0.2 | g |
| KH₂PO₄ | 0.2 | g |
| CaCl₂ | 0.02 | g |
| Trace metal solution (see Tab. 2) | 10 | ml |
| Wolfe's vitamin solution (see Tab. 2) | 10 | ml |
| Yeast Extract | 2 | g |
| Resazurin (2 g/L stock) | 0.5 | ml |
| 2-(N-morpholino)ethanesulfonic acid | 20 | g |
| (MES) | | |
| Reducing agent | 0.006-0.008% | (v/v) |
| Fructose | 5 | g |
| Sodium acetate | 0.25 | g |
| Fe (SO₄)₂(NH₄)₂•6H₂O | 0.05 | g |
| Nitriolotriacetic Acid | 0.05 | g |

| | |
|---|---|
| pH 5.7 | Adjusted with NaOH |

### Confirmation of Transformation Success:

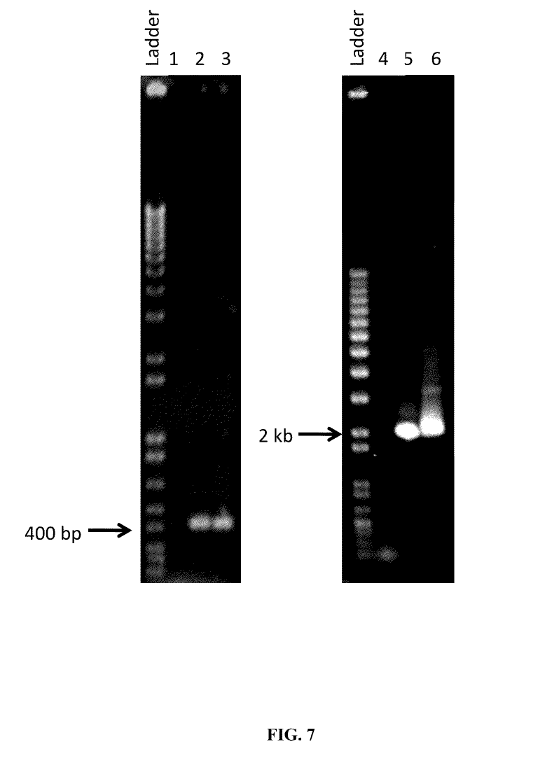

To verify the DNA transfer, a plasmid mini prep was performed from 10 ml culture volume using Zyppy plasmid miniprep kit (Zymo). PCR was performed with the isolated plasmid as template using primer pairs ermB-F (SEQ_ID NO. 20) plus ermB-R (SEQ_ID NO. 21), and SOE-GroESL-a-NdeI (SEQ_ID NO. 6) and SOE-GroESL-d-EcoRI (SEQ_ID NO. 9) to confirm the presence of the plasmid (FIG. 7; FIG. 12). PCR was carried out using iProof High Fidelity DNA Polymerase (Bio-Rad Labratories) and the following program: initial denaturation at 98° C. for 30 seconds, followed by 35 cycles of denaturation (98° C. for 10 seconds), annealing (55° C. for 30 seconds) and elongation (72° C. for 15-60 seconds), before a final extension step (72° C. for 10 minutes).

To confirm the identity of the clones, genomic DNA was isolated (see above) and a PCR was performed against the 16s rRNA gene using oligonucleotides fD1 (SEQ_ID NO. 22) and rP2 (SEQ_ID NO. 23) (Weisberg W A, Barns S M, Pelletier D A and Lane D J: 16S rDNA amplification for phylogenetic study. J Bacteriol 1991, 173: 697-703) and iNtRON Maximise Premix PCR kit (Intron Bio Technologies) with the following conditions: initial denaturation at 94° C. for 2 minutes, followed by 35 cycles of denaturation (94° C. for 20 seconds), annealing (55° C. for 20 seconds) and elongation (72° C. for 60 seconds), before a final extension step (72° C. for 5 minutes). Sequencing results confirmed 99.9% identity against the 16S rRNA gene of C. autoethanogenum (Y18178, GI:7271109)—(GenBank accession number, gene ID number).

Overexpression of GroESL Enhanced Ethanol Tolerance, Growth and Production of C. autoethanogenum DSM23693:

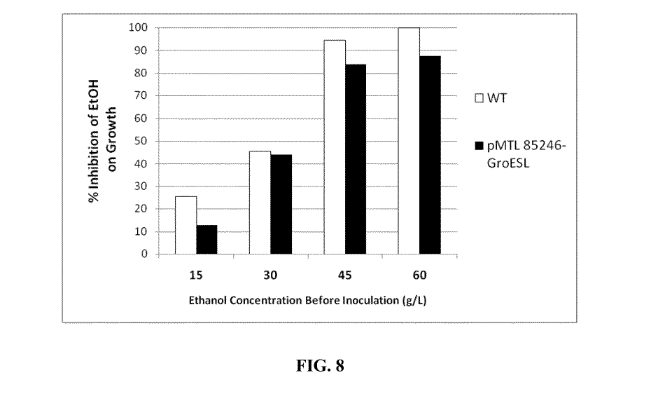

To investigate whether overexpression of GroESL enhances ethanol tolerance of C. autoethanogenum DSM23693, both wild-type (WT), i.e. parental microorganism and transformed strain carrying plasmid pMTL85246-GroESL were challenged with different concentrations of ethanol (FIG. 8).

Growth experiments in triplicates were carried out in 50 ml PETC media (Table 1) in serum bottles sealed with rubber stoppers and 30 psi steel mill gas (collected from New Zealand Steel site in Glenbrook, NZ; composition: 44% CO, 32% N₂, 22% CO₂, 2% H₂) in the headspace as sole energy and carbon source. Different amounts of anaerobized ethanol was added to the media prior to inoculation to achieve final ethanol concentrations of 15 g/L, 30 g/L, 45 g/L and 60 g/L (which was confirmed by HPLC). All cultures were inoculated to the same optical density using the same pre-culture for either wild-type (parental) or transformed strain. Changes in biomass were measured spectrophotometrically at 600 nm until growth ceased. The maximum biomass of each culture was compared with the unchallenged culture.

Cultures that overexpressed Heat shock protein/chaperonin complex GroESL were generally found to have an increased ethanol tolerance when compared to an unchallenged culture. While growth of the wildtype (parental) ceased after addition of 60 g/l ethanol completely, the strain overproducing GroESL (transformed) was still able to grow. The wild-type (parental) culture showed only 0.39 doubling when challenged with 45 g/l ethanol and biomass even dropped when 60 g/l ethanol was added, while the culture overproducing GroESL doubled 2.14 and respectively 1.27 times when challenged with 45 and respectively 60 g/l ethanol.

While the wild-type (parental) of C. autoethanogenum shows no growth at ethanol concentrations greater 50 g/l or 5% (w/v) in serum bottle experiments (FIGS. 1 and 8), the modified strain which overproduces Heat shock protein/chaperonin complex GroESL was surprisingly able to grow even in presence of 60 g/l or 6% (w/v) ethanol.

Furthermore, it was surprisingly found that ethanol production at high concentrations was increased in the modified strain which overproduces Heat shock protein/chaperonin complex GroESL over the wild-type (parental) of C. autoethanogenum as seen in Tab. 5. At an added ethanol concentration of around 45 g/L, the wild-type (parental) produced only 0.04 g/L ethanol, while the strain overproducing Heat shock protein/chaperonin complex GroESL produced 1.01 g/L (400% increase). At an added ethanol concentration of around 60 g/L, the wild-type (parental) produced only 0.14 g/L ethanol, while the strain overproducing Heat shock protein/chaperonin complex GroESL produced 1.61 g/L (250% increase).

In addition, it was surprisingly found that when overproducing Heat shock protein/chaperonin complex GroESL, ethanol production was best at high ethanol concentration, while in the wild-type (parental) the highest ethanol production was found when no ethanol was added. In wild-type (parental) 0.72 g/L was the highest ethanol production found, when no ethanol was added. All cultures of the wild-type in which ethanol was added, produced less than 0.14 g/L. The modified strain overproducing Heat shock protein/chaperonin complex GroESL, produced the same amount of ethanol (0.7 g/L) as the wild type (parental) when no ethanol was added, but in contrast to the wild-type (parental) produced even higher levels of ethanol (over 1 g/L) when challenged with high ethanol concentrations over 45 g/L.

TABLE 5
Ethanol concentrations at inoculation and after 40 hours
of growth in both the wild-type (parental) strain of C. autoethanogenum
DSM23693 and the modified strain overproduces Heat shock
protein/chaperonin complex GroESL:

| | | |
|---|---|---|
| Ethanol at | Total ethanol after | Ethanol |
| inoculation [g/L] | 40 h [g/L] | produced [g/L] |

Wild-type (parental):

| | | |
|---|---|---|
| 0.09 | 0.81 | 0.72 |
| 14.58 | 14.59 | 0.01 |
| 28.19 | 28.17 | −0.02 |
| 44.89 | 44.93 | 0.04 |
| 55.19 | 55.33 | 0.14 |

Strain harboring GroESL plasmid
(modified or transformed strain)

| | | |
|---|---|---|
| 0.07 | 0.77 | 0.7 |
| 15.45 | 15.4 | −0.05 |
| 30.87 | 31.09 | 0.22 |
| 45.65 | 46.66 | 1.01 |
| 56.84 | 58.46 | 1.62 |

qRT-PCR experiments were performed to confirm over-expression of the groESL genes compared to the wild-type (parental) strain. Normalized mRNA levels of the groESL operon was found 10.7 fold higher in the overexpression strain harbouring plasmid pMTL85246-GroESL compared to the wild-type (parental) strain at mid logarithmic growth.

An unchallenged 50-ml overnight culture of each wild-type (parental) and strain harbouring over-expression plasmid pMTL85246-GroESL was harvested by centrifugation (6,000×g, 5 min, 4° C.). RNA was isolated from the same amount of cells by suspending the pellet in 100 μL of lysozyme solution (50,000 U lysozyme, 0.5 μL 10% SDS, 10 mM Tris-HCl, 0.1 mM EDTA; pH 8). After 5 min, 350 μL of lysis buffer (containing 10 μL of 2-mercaptoethanol) was added. The cell suspension was mechanistically disrupted by passing five times through an 18-21 gauge needle. RNA was then isolated using PureLink™ RNA Mini Kit (Invitrogen) and eluted in 100 μL of RNase-free water. The RNA was checked via PCR and gel electrophoresis and quantified spectrophotometrically, and treated with DNase I (Roche) if necessary. Quality and integrity of RNA was checked with an BioAnalyzer 2100 (Agilent Technologies) and Qubit (Invitrogen). The reverse transcription step was carried out using SuperScript III Reverse Transcriptase Kit (Invitrogen). qRT-PCR reactions were performed in triplicates in a MyiQ Single Colour Real-Time PCR Detection System (Bio-Rad Labratories). The reaction volume was 15 μL with 25 ng of cDNA template, 67 nM of each primer (Table 6), and 1× iQ SYBR Green Supermix (Bio-Rad Labratories, Hercules, Calif. 94547, USA) and the following conditions were used: 95° C. for 3 min, followed by 40 cycles of 95° C. for 15 s, 55° C. for 15 s and 72° C. for 30 s. A melting-curve analysis was performed immediately after completion of the qRT PCR (38 cycles of 58° C. to 95° C. at 1° C./s), for detection of primer dimerisation or other artifacts of amplification. Two housekeeping genes (Guanylate kinase and formate tetrahydrofolate ligase) were included for each cDNA sample for normalization. Derivation of relative gene expression was conducted using Relative Expression Software Tool (REST©) 2008 V2.0.7 (38). Dilution series of cDNA spanning 4 log units were used to generate standard curves and the resulting amplification efficiencies to calculate concentration of mRNA.

TABLE 6
Oligonucleotides for qRT-PCR

| | Oligonu- | | SEQ |
|---|---|---|---|
| | cleotide | DNA Sequence | ID |
| Target | Name | (5′ to 3′) | NO. |
| Guanylate | GnK-F | TCAGGACCTTCTGGAACTGG | 29 |
| kinase | GnK-R | ACCTCCCCTTTTCTTGGAGA | 30 |
| Formate | FoT4L-F | CAGGTTTCGGTGCTGACCTA | 31 |
| tetrahydro- | FoT4L-R | AACTCCGCCGTTGTATTTCA | 32 |
| folate | | | |
| ligase | | | |
| GroESL | GroESL- | AACTACGAAGAGCGGTATTGT | 33 |
| | RT-F | TTTA | |
| | GroESL | ACTTCTTTTCCATCTACTGTTC | 34 |
| | RT-R | CAC | |

Overexpression of GroESL Enhanced Ethanol Tolerance and Growth of C. autoethanogenum DSM10061:

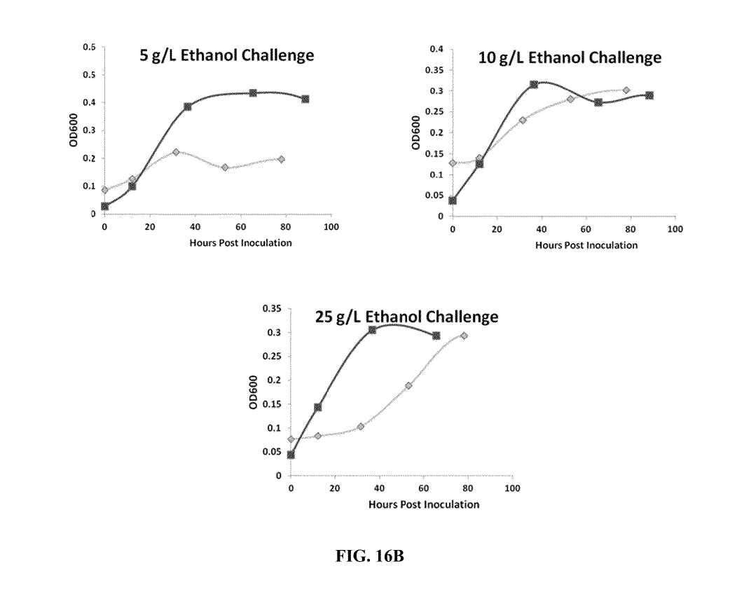

To demonstrate that GroESL chaperon overproduction has a positive effect on ethanol tolerance in carboxydotrophic acetogens, another strain of C. autoethanogenum, DSM10061, was modified with the GroESL expression plasmid and challenged with ethanol. While the C. autoethanogenum DSM23693 strain was found to be tolerant to 25 g/L of ethanol naturally, the C. autoethanogenum DSM10061 strain is unable to grow at that concentration (example 1, FIG. 1, FIG. 16). When however, chaperon GroES and GroEL were overproduced, this strain was able to grow in presence of 25 g/L and even at 50 g/L (FIG. 16).

The experiment was first conducted as described above using PETC medium and autotrophic conditions with CO as substrate (FIG. 16a) and afterwards repeated using a different growth medium and heterotrophic conditions with fructose as substrate to rule out the effect of the growth media and substrate (FIG. 16b). A plasmid control was included. Both plasmid control (pMTL83157) transformants and chaperone over-expressing (pMTL83156-groESL) transformants were cultured anaerobically in MMYF medium (Error! Reference source not found.) supplemented with freshly prepared 7.5 μg/mL thiamphenicol (final concentration). For ethanol challenge assay, 500 μL of two day old cultures were inoculated into each of five 60 mL serum bottles containing 20 mL of selective MMYF medium and incubated at 37° C. without agitation. After 12 hours of incubation, anaerobic ethanol was added to the cultures to achieve final concentrations of 5 g/L, 10 g/L, 25 g/L and 50 g/L. For each transformants, one serum bottle culture was not challenged with ethanol and served as “uninhibited control”. Static incubation at 37° C. was allowed for 80-100 hours and the growth was monitored by measuring the optical density at a wavelength of 600 nm using Jenway 7300 spectrophotometer. Microscope examinations were routinely carried out.

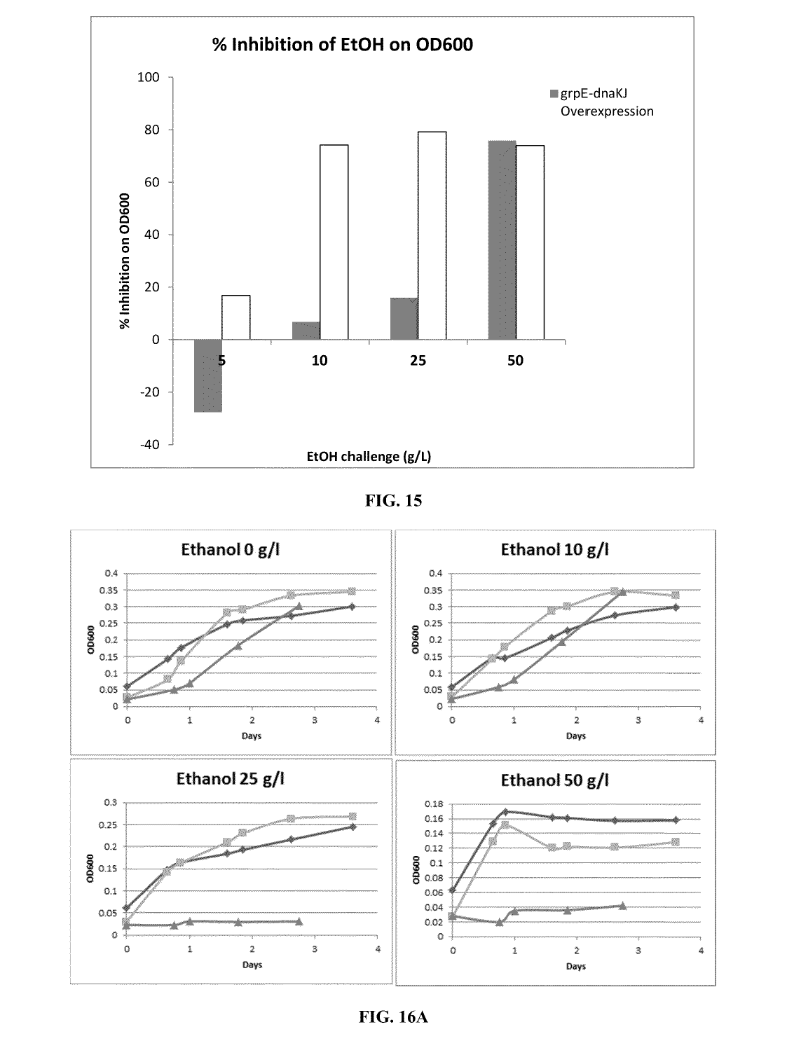

The over-expression of groESL in C. autoethanogenum DSM10061 resulted in significantly earlier growth relative to plasmid control when challenged with 5 g/L, 10 g/L and 25 g/L ethanol at 12 hour post inoculation (FIGS. 15 and 16 b). For instance, at 5 g/L of ethanol challenge, the groESL over-expressing transformant reached OD₆₀₀ of 0.38 at 37 h post inoculation whereas the plasmid control strain took more than 102 h to exceed OD₆₀₀ 0.25 (FIG. 15). When challenged with 25 g/L ethanol, the groESL over-expressing strain reached OD₆₀₀ of 0.31 at 37 h post inoculation, in comparison to the plasmid control strain that only reached OD₆₀₀ of 0.29 at 78 h post inoculation (Error! Reference source not found.C). At all levels of ethanol challenge, the strain overproducing chaperons GroES and GroEL resulted in higher biomass.

TABLE 8
MMYF Medium^{a}

| | | |
|---|---|---|
| | Concentration | Final |
| | in Stock | Concentration in |
| Stock Solution Component | Solution (g/l) | MMY Medium (g/l) |

Macronutrients (50x)

| | | |
|---|---|---|
| NH₄Cl | 50 | 1 |
| NaCl | 40 | 0.8 |
| KCl | 5 | 0.1 |
| KH₂PO₄ | 5 | 0.1 |
| MgSO₄•7H₂O | 10 | 0.2 |
| CaCl₂•2H₂O | 2 | 0.04 |

Acidic trace element solution (1000x)

| | | |
|---|---|---|
| HCl | 50^{b} | 0.05^{b} |
| H₃BO₃ | 0.1 | 0.0001 |
| MnCl₂•4H₂O | 0.23 | 0.00023 |
| FeCl₂•4H₂O | 0.78 | 0.00078 |
| CoCl₂•6H₂O | 0.103 | 0.000103 |
| NiCl₂•6H₂O | 0.602 | 0.000602 |
| ZnCl₂ | 0.078 | 0.000078 |
| CuSO₄•5H₂O | 0.05 | 0.00005 |
| AlK(SO₄)₂•12H₂O | 0.05 | 0.00005 |

Basic trace element solution (1000x)

| | | |
|---|---|---|
| NaOH | 10^{b} | 0.01^{b} |
| Na₂SeO₃ | 0.058 | 0.000058 |
| Na₂WO₄ | 0.053 | 0.000053 |
| Na₂MbO₄•2H₂O | 0.052 | 0.000052 |

B-vitamin solution (1000x)

| | | |
|---|---|---|
| p-aminobenzoate | 0.1 | 0.0001 |
| riboflavin | 0.1 | 0.0001 |
| thiamine | 0.2 | 0.0002 |
| nicotinate | 0.2 | 0.0005 |
| pyridoxin | 0.5 | 0.0001 |
| calcium-D-pantothenate | 0.1 | 0.0001 |
| cyanocobalamin | 0.1 | 0.0001 |
| d-biotin | 0.02 | 0.00002 |
| folate | 0.05 | 0.00005 |
| lipoate/thioctic acid | 0.05 | 0.00005 |
| MES (2-(N-morpholino)ethane- | | 5 |
| sulfonic acid) | | |
| D-fructose | | 7.2 |
| Sodium formate | | 1 |
| NaOH | | 10^{b} |
| Resazurin (2000x) | 2 | 0.001 |
| Cysteine stock solution (100x) | 40 | 0.4 |

Titanium NTA stock solution (200x)

| | | |
|---|---|---|
| NTA (Nitrilo triacetic acid) | 76.4 | 0.382 |
| NaOH | 53.3 | 0.2665 |
| Na₂CO₃ | 28.3 | 0.1415 |
| TiCl₃ | 62.7^{b} | 0.3135^{b} |

^{a}A litre of MM was made by adding 5 g MES, 7.2 g D-fructose, 1 g sodium formate, 2 ml 5M NaOH, 1 ml Acidic trace solution (1000x), 1 ml Basic trace solution (1000x), 20 ml Macronutrient solution (50x), 0.5 ml Resazurin stock solution, adjusted to pH 5.8 using HCl and final volume of 1 l, followed by sterilization via autoclave. Prior to inoculation, 10 ml of filter-sterilized anaerobic cysteine stock solution (100x) and 5 ml of filter sterilized anaerobic Titanium NTA stock solution (200x) were added to reduce the medium.
^{b}Units in mM

Heterologous Expression of GroESL in C. ljungdahlii DSM13528 for Enhanced Ethanol Tolerance and Effect of Different Promoters:

To demonstrate that the effect of overproduction of GroES and GroEL was also beneficial to other ethanol producing carboxydotrophic acetogens, C. ljungdahlii, the C. autoethanogenum groESL genes were heterologously expressed in C. ljungdahlii and also with this organism a positive effect on ethanol tolerance was observed. The experiment was performed as described above in MMYF media.

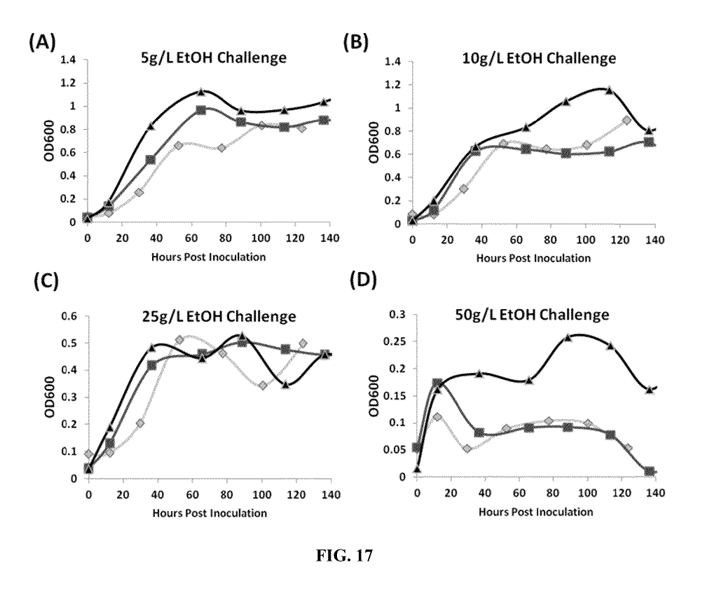

When challenged with 5 g/L of ethanol (final concentration) after 12 hours of incubation, the over-expressing transformants reached significantly higher OD₆₀₀ when compared to plasmid control in the first 66 hours. At this time point, groESL over-expressing transformants reached OD₆₀₀ of 0.97, respectively, while the plasmid control recorded OD₆₀₀ of ˜0.66 (FIG. 17). At the 10 g/L ethanol challenge level, the GroES and GroEL chaperone over-expressing transformants reached OD₆₀₀ of ˜0.6 significantly earlier than plasmid control (FIG. 17). At 25 g/L ethanol challenge level, groESL over-expressing transformant reached OD₆₀₀ of 0.42, at 36 h post inoculation, in contrast to plasmid control OD₆₀₀ of 0.20 at 30 h post inoculation (FIG. 17).

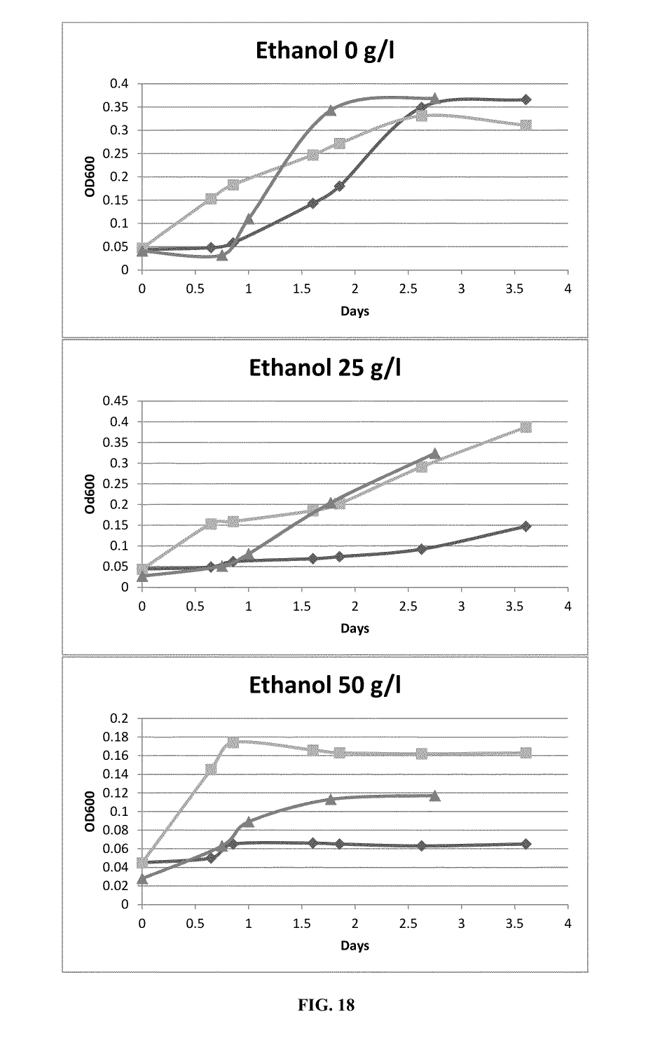

In addition, the effect of different promoter sequences was evaluated. The two strong C. autoethanogenum promoters of pyruvate:ferredoxin oxidoreductase and phosphotransacetylase/acetate kinase operon were used. While both are strong, constitutive promoters, the GroESL expression construct with the phosphotransacetylase/acetate kinase operon promoter didn't enhance ethanol tolerance in C. ljungdahlii over the wild-type, whereas ethanol tolerance was significantly enhanced in the construct with the pyruvate:ferredoxin oxidoreductase promoter as seen in FIG. 18. This again shows that some promoters will enhance ethanol tolerance better for one microorganism versus another microorganism and their effect is not easily predicted.

### Example 3

### Genetic Modification of Clostridium ljungdahlii DSM13528 and C. autoethanogenum DSM10061 with Chaperons GrpE, DnaK and DnaJ for Improved Ethanol Tolerance

Since overproduction of chaperons GroES and GroEL was surprisingly found to have a positive effect on ethanol tolerance, growth and ethanol production of carboxydotrophic acetogens, overproduction of another chaperon complex consisting of Hsp70 chaperon (DnaK) (Seq ID 38), Hsp40 chaperon (DnaJ) (Seq ID 40), and heat shock protein (GrpE) (Seq ID 36) was overproduced in C. autoethanogenum DSM10061 and C. ljungdahlii DSM13583. As with GroESL, it was found that these chaperons have a beneficial effect on ethanol tolerance and growth of the culture in both carboxydotrophic acetogens.

Construction of a grpE-dnaKJ Expression Plasmid:

Chaperone genes grpE (Seq ID 35) dnaK (Seq. ID 37) and dnaJ (Seq ID 39) were amplified from genomic DNA of C. autoethanogenum (example 1) by PCR with oligonucleotides in Table 7 using iProof High Fidelity DNA Polymerase (Bio-Rad Laboratories) and the following program: initial denaturation at 98° C. for 30 seconds, followed by 32 cycles of denaturation (98° C. for 10 seconds), annealing (50-62° C. for 30-120 seconds) and elongation (72° C. for 45 seconds), before a final extension step (72° C. for 10 minutes). For amplifications of 16s rRNA and detection of plasmid, Phusion High-Fidelity DNA Polymerase (NEB) was used.

TABLE 7
Oligonucleotides used for cloning

| | Oligonu- | | SEQ_ |
|---|---|---|---|
| | cleotide | DNA Sequence | ID |
| Target | Name | (5′ to 3′) | NO. |
| grpE-dnaK-dnaJ | grpE- | GCCATATGTTAAAGGATAAAGGT | 42 |
| operon of C. | NdeI-F | GATAATG | |
| autoethanogenum | | | |
| grpE-dnaK-dnaJ | dnaJ- | CCGAGCTCTATTAGTGGTGATGT | 43 |
| operon of C. | SacI-R | TTAAG | |
| autoethanogenum | | | |

Construction of Expression Plasmids and Control Plasmid were Performed in E. Coli DH5α-T1^{R} (Invitrogen) and E. coli ABLE K (Stratagene).

The chaperone operon grpE-dnaK-dnaJ (Seq. ID 41) was amplified from genomic DNA of C. autoethanogenum using primers grpE-NdeI-F and dnaJ-SacI-R (Table 7). The resulting 4050 bp PCR product and plasmid pMTL83156 (Seq. ID 46) were then digested with restriction enzymes NdeI and SacI, followed by ligation to generate plasmid pMTL83156-grpE-dnaK-dnaJ (Error! Reference source not found. FIG. 10; SEQ_ID. 47). The whole chaperone operon together with promoter P_{pfor} was sequenced using cloning primers (Table)) and sequencing primers (Table) to confirm the identity of the cloned DNA fragments (Error! Reference source not found.).

Methylation and Transformation of zrpE-dnaKJ Expression Plasmid and Control Plasmid into C. autoethanogenum and C. ljungdahlii

Chaperone over-expressing plasmid and pMTL83157 plasmid control were introduced into the clostridial hosts (C. autoethanogenum and C. ljungdahlii) as described in example 1.

### Confirmation of Transformation Success:

Successful transformed colonies were obtained and selected for using the antibiotic thiamphenicol (7.5 μg/mL final concentration) and they were re-streaked onto the same selective media at least once for purity. The identities of the transformed clostridial hosts were validated by 16s rRNA sequencing using primer pairs Univ-0027-F and Univ-1492-R (Table 9). The presence of the introduced plasmids in transformants were detected by first performing plasmid miniprep using QIAGEN Plasmid mini kit followed by PCR using primers repHf and catR (Table) (Error! Reference source not found.). Due to the strong nuclease activities of many Clostridia, miniprep plasmids harvested from transformed Clostridia were first transformed into E. coli strain XL1-Blue MRF' Kan (Stratagene) or Top10 (Invitrogen) to “rescue” the plasmids before restriction digest analyses (PmeI and AscI) were performed to confirm the identity of the transformed plasmids (Error! Reference source not found.). Confirmed transformants were stored frozen in final glycerol concentration of 15% (v/v) in −80° C. freezer.

TABLE 9
Oligonucleotides used for DNA sequencing
and detection of plasmids

| | | SEQ_ |
|---|---|---|
| Oligonucleotide | DNA Sequence | ID |
| Name | (5′ to 3′) | NO. |
| M13 Forward | TGTAAAACGACGGCCAGT | 13 |
| (−20) | | |
| M13 Reverse | CAGGAAACAGCTATGACC | 14 |
| grpE-seq1 | CATCAGTAGTATCATTCCAGGC | 49 |
| grpE-seq2 | AAATAAGATCATATTAGTTGGTGG | 50 |
| grpE-seq3 | GGAATTACATCTAAAATATATAGTCAG | 51 |
| Univ-0027-F | GCGAGAGTTTGATCCTGGCTCAG | 52 |
| Univ-1492-R | CGCGGTTACCTTGTTACGACTT | 53 |
| repHf | AAGAAGGGCGTATATGAAAACTTGT | 54 |
| catR | TTCGTTTACAAAACGGCAAATGTGA | 55 |

Overexpression of GrpE, DnaK, DnaJ Overproduction in C. autoethanogenum DSM10061:

To test the effects of GrpE, DnaK, DnaJ chaperone over-production on the ability of C. autoethanogenum and C. ljungdahlii to tolerate ethanol, both plasmid control (pMTL83157) transformants and chaperone over-expressing (pMTL83156-grpE-dnaK-dnaJ) transformants were cultured anaerobically in MMYF medium (table 8) supplemented with freshly prepared 7.5 μg/mL thiamphenicol (final concentration). For ethanol challenge assay, 500 μL of two day old cultures were inoculated into each of five 60 mL serum bottles containing 20 mL of selective MMYF medium and incubated at 37° C. without agitation. After 12 hours of incubation, anaerobic ethanol was added to the cultures to achieve final concentrations of 5 g/L, 10 g/L, 25 g/L and 50 g/L. For each transformants, one serum bottle culture was not challenged with ethanol and served as “uninhibited control”.

In the earlier growth phase (up to 102 h post inoculation) of C. autoethanogenum, the over-expression of grpE-dnaK-dnaJ allowed the transformants to grow in a manner that is significantly less inhibited by ethanol challenge at 5 g/L, 10 g/L and 25 g/L levels (Error! Reference source not found.19). At 10 g/L and 25 g/L ethanol challenge levels, the plasmid control strain showed OD₆₀₀ inhibition of 74% and 79%, respectively at 102 h post inoculation.

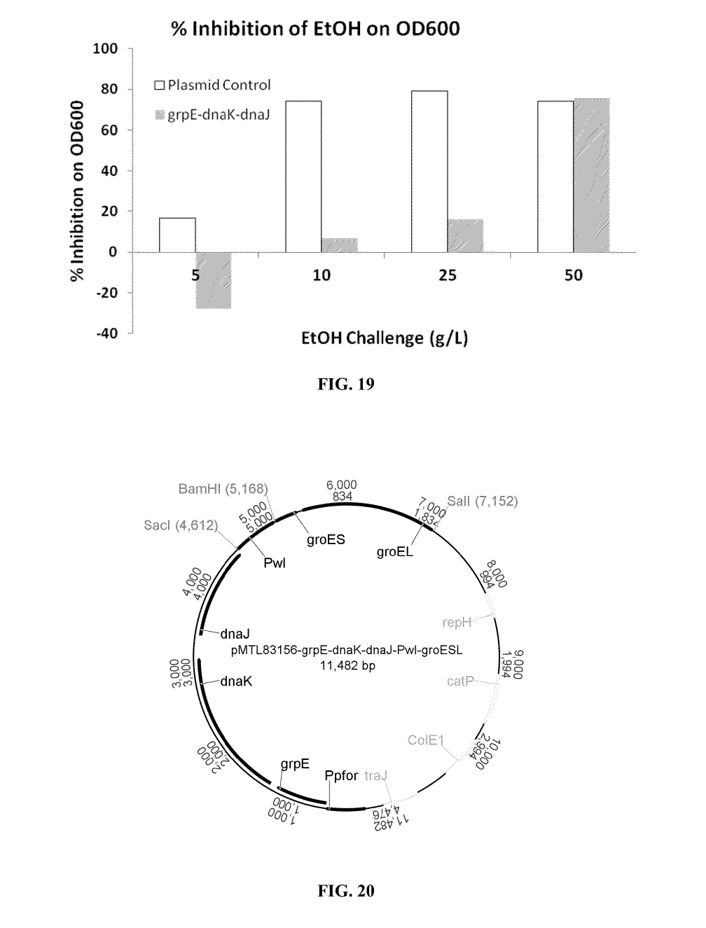

In contrast, the grpE-dnaK-dnaJ over-expressing transformant only showed OD₆₀₀ inhibition of 7% and 16% at 10 g/L and 25 g/L ethanol challenge, respectively, at the same time point (FIG. 16). Surprisingly, the addition of 5 g/L ethanol (final concentration) to the grpE-dnaK-dnaJ over-expressing transformant not only showed no OD₆₀₀ inhibition but increased OD₆₀₀ by 28% relative to unchallenged culture (FIG. 19).

Heterologous Expression of GroESL in C. ljungdahlii DSM 13583:

The same ethanol challenge experiment as with C. autoethanogenum was also performed with C. ljungdahlii DSM13583, the wild-type and a mutant strain heterologously expressing grpE-dnaK-dnaJ on a plasmid.

When challenged with 5 g/L of ethanol (final concentration) after 12 hours of incubation, the grpE-dnaK-dnaJ (as the groESL over-expressing) transformants reached significantly higher OD₆₀₀ when compared to plasmid control in the first 66 hours. At this time point, the grpE-dnaK-dnaJ (as the groESL over-expressing) transformants reached OD₆₀₀ of 1.12 (0.97 for groESL over-expression) while the plasmid control recorded only an OD₆₀₀ of ˜0.66 (FIG. 17a). At the 10 g/L ethanol challenge level, both chaperone over-expressing transformants reached OD₆₀₀ of ˜0.6 significantly earlier than plasmid control (FIG. 17b). Furthermore, the grpE-dnaK-dnaJ over-expressing transformants reached a much higher OD₆₀₀ of 1.15 at 114 h post inoculation relative to plasmid control OD₆₀₀ of 0.67 at 101 h post inoculation (FIG. 17b). At 25 g/L ethanol challenge level, grpE-dnaK-dnaJ (as the groESL over-expressing) transformant reached OD₆₀₀ of 0.48 (0.42 for groESL), at 36 hrs. post inoculation, in contrast to plasmid control OD₆₀₀ of 0.20 at 30 h post inoculation (FIG. 17c).

At 50 g/L ethanol challenge at 12 hour post inoculation, both plasmid control and groESL over-expressing transformants showed reduction in OD₆₀₀, whereas grpE-dnaK-dnaJ over-expressing transformant showed an increase in OD₆₀₀ of 0.096 (FIG. 17d).

In addition to earlier growth relative to plasmid control, ethanol challenge at 5 g/L and 10 g/L also stimulated the grpE-dnaK-dnaJ transformants to achieve higher OD₆₀₀ than plasmid controls in the first 120 hours of growth (FIGS. 17a and 17b).

### Example 4

### Combination of groESL and grpE-dnaKJ to Further Enhance Ethanol Tolerance

Since the individual over-expression of chaperone groESL or grpE-dnaK-dnaJ in carboxydotrophic acetogens C. autoethanogenum and C. ljungdahlii resulted in significant improvements in ethanol tolerance relative to plasmid controls, one can clone and over-express both chaperone complexes in the same plasmid. Another strong promoter, such as the promoter of Wood-Ljungdahl cluster (Seq. ID 24) or promoter of Rnf complex (Seq ID 26), can be introduced between the two chaperone complex sequences to ensure strong expression of the downstream genes. As an example, the promoter P_{wl} can be cloned into pMTL83156-grpE-dnaK-dnaJ using restriction sites SacI and BamHI, followed by the cloning of groESL using restriction sites BamHI and SalI, generating plasmid pMTL83156-grpE-dnaK-dnaJ-P_{WL}-groESL (Error! Reference source not found.; Seq_ID_{—}59). Furthermore, clippase B (clpB) (SEQ_ID_{—}49), which is hypothesized to act as part of the grpE-dnaK-dnaJ multi-chaperone system to disaggregate proteins and allow their refolding can also be cloned into the same plasmid to further enhance alcohol tolerance.

The newly constructed multi-chaperone over-expressing plasmids can be transformed into acetogens such as C. autoethanogenum and C. ljungdahlii via electroporation as previously described. Successful transformants can be selected by using 7.5 μg/mL thiamphenicol and screened for the presence of over-expression plasmids by “plasmid rescue” or PCR using primers repHf and catR (Table). The identity of the transformants can be verified by performing genomic DNA extraction and PCR followed by sequencing of 16s rRNA. Validated transformants along with plasmid controls can then be subjected to alcohol challenge as previously described to investigate the effects of multi-chaperone over-expression on alcohol tolerance.

Finally, these chaperone complexes can be integrated into the genome via homologous recombination to engineer a stable recombinant strain without the need of antibiotic supplementation. Given the positive effects of over-expression of individual chaperone on ethanol tolerance, it is anticipated that the combined over-expression of multi-chaperone system should be able to further improve the alcohol tolerance of the recombinant microorganisms to about 100 g/L or 10% (w/v).

### Example 5

### Enhance Ethanol Tolerance of C. ragsdalei

Since over-expression and heterologous expression of chaperon complexes groESL and grpE-dnaKJ has been shown to enhance ethanol tolerance in three strains of carboxydotrophic acetogens, the same strategy can be deployed on other carboxydotrophic acetogens such as C. ljungdahlii ERI-2 (ATCC 55380) (U.S. Pat. No. 5,593,886), C. ljungdahlii C-01 (ATCC 55988) (U.S. Pat. No. 6,368,819), C. ljungdahlii O-52 (ATCC 55989) (U.S. Pat. No. 6,368,819), or “C. ragsdalei P11^{T}” (ATCC BAA-622) (WO 2008/028055), and “C. coskatii” (US patent 2011/0229947), which all have similar properties.

Chaperon expression plasmids described above such as pMTL83156-groESL, pMTL83156-grpE-dnaK-dnaJ, or pMTL83156-grpE-dnaK-dnaJ-P_{WL}-groESLcan be transformed into C. ljungdahlii ERI-2 (ATCC 55380), C. ljungdahlii C-01 (ATCC 55988), C. ljungdahlii O-52 (ATCC 55989), or “C. ragsdalei P11^{T}” (ATCC BAA-622), or “C. coskatii” using methods described above which should result in enhanced ethanol tolerance of at least 50 g/L or 5% (w/v) or higher.

### Example 6

### Combination of groESL and grpE-dnaKJ with Other Chaperons to Further Enhance Ethanol Tolerance

In addition to chaperons Cpn10 chaperonin (GroES), Cpn60 chaperonin (GroEL), Hsp70 chaperon (DnaK), Hsp40 chaperon (DnaJ), and heat shock protein (GrpE) which have been shown to enhance ethanol tolerance in acetogenic carboxydotrophes C. autoethanogenum and C. ljungdahlii, other chaperons such as protein disaggregation chaperone (ClpB), class III stress response-related ATPase (ClpC), ATP-dependent serine protease (ClpP), heat shock protein (Hsp18), heat shock protein (Hsp90) can be used to enhance ethanol tolerance, growth and ethanol production further.

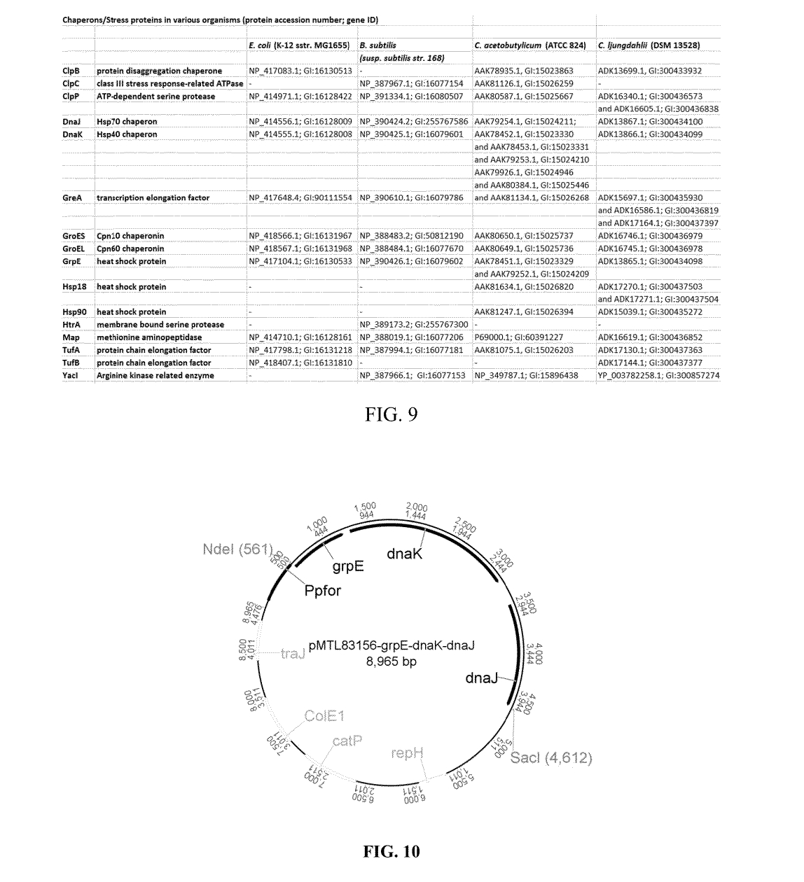

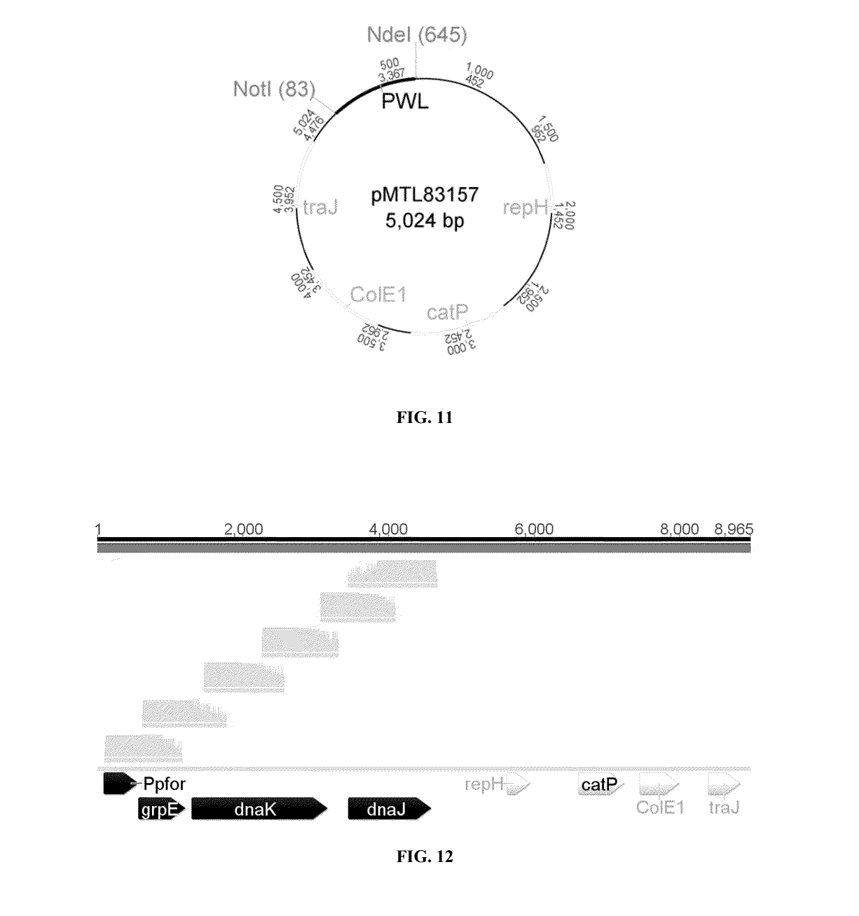

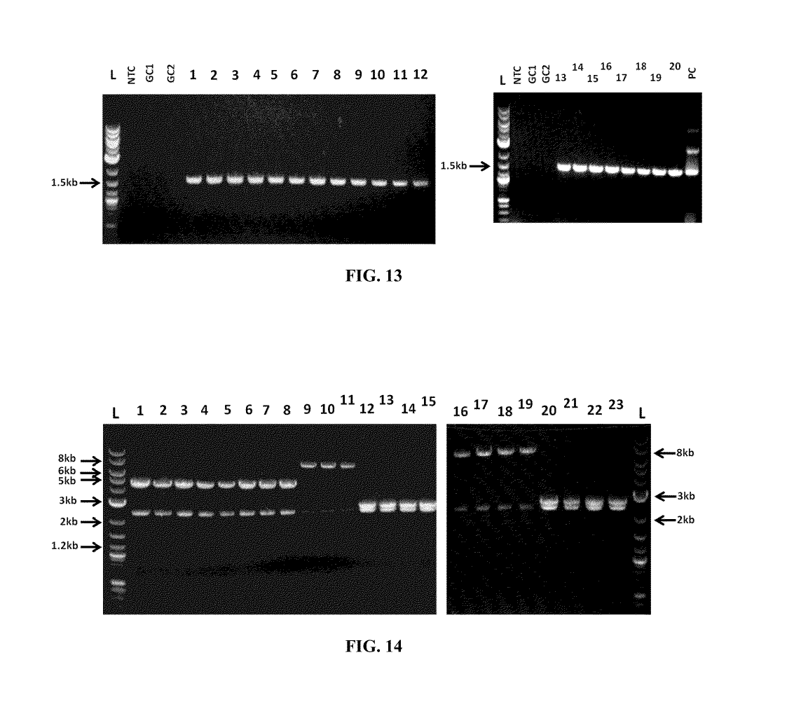

These genes can be added into plasmid pMTL83156-grpE-dnaK-dnaJ-P_{WL}-groESL or integrated into the genome as described in example 4. Table 10 provides the necessary sequence information from C. autoethanogenum, and in FIG. 9 are Genbank numbers for similar chaperons from C. ljungdahlii and other organisms given that may be used. Sequences can be either amplified from the genome or synthesized. By overexpressing these chaperons, acetogenic carboxydotropic strains should be able to tolerate about 150 g/L or 15% (w/v) ethanol.

TABLE 10
Additional chaperons

| | Nucleic acid | Amino acid |
|---|---|---|
| Chaperon | sequence | sequence |
| protein disaggregation chaperone (ClpB) | 49 | 50 |
| class III stress response-related | 51 | 52 |
| ATPase (ClpC) | | |
| ATP-dependent serine protease (ClpP) | 53 | 54 |
| heat shock protein (Hsp18) | 55 | 56 |
| heat shock protein (Hsp90) | 57 | 58 |

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. Titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1. A recombinant carboxydotrophic acetogenic microorganism modified from a parental carboxydotrophic acetogenic microorganism to overexpress at least one enzyme selected from the group consisting of protein disaggregation chaperone (ClpB), class III stress response-related ATPase (ClpC), ATP-dependent serine protease (ClpP), Hsp70 chaperon (DnaK), Hsp40 chaperon (DnaJ), transcription elongation factor (GreA), Cpn10 chaperonin (GroES) enzyme, or Cpn60-chaperonin (GroEL) enzyme, heat shock protein (GrpE), heat shock protein (Hsp 18), heat shock protein (Hsp90), membrane bound serine protease (HtrA), methionine aminopeptidase (Map), protein chain elongation factor (TufA), protein chain elongation factor (TufB), Arginine kinase related enzyme (YacI) as compared to a parental carboxydotrophic acetogenic microorganism not modified to overexpress the at least one enzyme wherein overexpression of the at least one enzyme is by transformation with a polynucleotide encoding the at least one enzyme, increasing copy number of a polynucleotide encoding the at least one enzyme, or an exogenous promoter operably linked to a polynucleotide encoding the at least one enzyme, and wherein the recombinant carboxydotrophic acetogenic microorganism has increased tolerance to ethanol compared to a parental carboxydotrophic acetogenic microorganism not modified to overexpress the at least one enzyme.

2. The recombinant carboxydotrophic acetogenic microorganism of claim 1 wherein the recombinant carboxydotrophic acetogenic microorganism is tolerant of ethanol concentrations of at least 5.5% by weight of fermentation broth.

3. The recombinant carboxydotrophic acetogenic microorganism of claim 1 wherein the recombinant carboxydotrophic acetogenic microorganism is tolerant of ethanol concentrations of at least 6% by weight of fermentation broth.

4. The recombinant carboxydotrophic acetogenic microorganism of claim 1 wherein the recombinant carboxydotrophic acetogenic microorganism is tolerant of ethanol concentrations from about 3% to about 15% by weight of fermentation broth.

5. The recombinant carboxydotrophic acetogenic microorganism of claim 4 wherein the recombinant carboxydotrophic acetogenic microorganism is tolerant of ethanol concentrations from about 5.5% to about 15% by weight of fermentation broth.

6. The recombinant carboxydotrophic acetogenic microorganism of claim 1, wherein the recombinant carboxydotrophic acetogenic microorganism comprises an exogenous promoter operably linked to a polynucleotide encoding the at least one enzyme.

7. The recombinant carboxydotrophic acetogenic microorganism of claim 1, wherein the recombinant carboxydotrophic acetogenic microorganism is transformed with a polynucleotide encoding the at least one enzyme.

8. The recombinant carboxydotrophic acetogenic microorganism of claim 1, wherein the recombinant carboxydotrophic acetogenic microorganism is generated from the parental carboxydotrophic acetogenic microorganism selected from the group consisting of Clostridium autoethanogenum, Clostridium ljungdahlii, Clostridium ragsdalei and Clostridium coskatii.

9. The recombinant carboxydotrophic acetogenic microorganism of claim 8, wherein the recombinant carboxydotrophic acetogenic microorganism is generated from the parental carboxydotrophic acetogenic microorganism Clostridium autoethanogenum.

10. The recombinant carboxydotrophic acetogenic microorganism of claim 9, wherein the recombinant carboxydotrophic acetogenic microorganism is generated from the parental carboxydotrophic acetogenic microorganism Clostridium autoethanogenum DSM23693.

11. The recombinant carboxydotrophic acetogenic microorganism of claim 9, wherein the recombinant carboxydotrophic acetogenic microorganism is generated from the parental carboxydotrophic acetogenic microorganism Clostridium autoethanogenum DSM100611.

12. The recombinant carboxydotrophic acetogenic microorganism of claim 8, wherein the recombinant carboxydotrophic acetogenic microorganism is generated from the parental carboxydotrophic acetogenic microorganism Clostridium ljundahlii.

13. The recombinant carboxydotrophic acetogenic microorganism of claim 1, wherein the recombinant carboxydotrophic acetogenic microorganism is generated from the parental carboxydotrophic acetogenic microorganism Clostridium ljungdahlii DSM13583.

14. The recombinant carboxydotrophic acetogenic microorganism claim 1 wherein the at least one enzyme is GroES or GroEL.


SEQUENCE LISTING (61 sequences)

SEQ ID NO: 1 - protein, 94 aa, Clostridium autoethanogenum
MKIRPLGDRVVIKKLEAEETTKSGIVLPGSAKEKPQEAEVVAVGIGGTVDGKEVKMEVKVGDKVLFSKYAGNEVKIDAQEYTILKQDDILAIIE

SEQ ID NO: 2 - protein, 544 aa, Clostridium autoethanogenum
MAKSILFGEDARKSMQEGVNKLANAVKVTLGPKGRNVVLDKKFGSPLITNDGVTIAKEIELEDPYENMGAQLVKEVATKTNDVAGDGTTTATLLAQAIIREGLKNVTAGANPMLIRQGIKMAVDKAVEEIKKVSTTVKGKEDIARIAAISASDEEIGKLIADAMEKVGNEGVITVEESKTMGTELDVVEGMQFDRGYLSPYMVTDSEKMEAAIEDPYILITDKKISNIQDILPLLEKIVQQGKKLLIIAEDVEGEALATLVVNKLRGTFTCVAVKAPGFGDRRKEMLQDIAILTGGQVISEELGRDLKEAELEDLGRAESVKIDKENTTIVNGRGDKKAIADRVSQIKVQIEETTSDFDKEKLQERLAKLAGGVAVVKVGAATETELKEKKLRIEDALAATKAGVEEGMGPGGGTAYINAIPEVEKLTSDVPDVKVGIDIIRKALEEPVRQIASNAGVEGSVIIQKVRNSEIGVGYDALKGEYVNMVEKGIVDPTKVTRSALQNAASVAATFLTTEAAVADIPEKAPAGPAAGAPGMGGMEGMY

SEQ ID NO: 3 - DNA, 285 bp, Clostridium autoethanogenum
atgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtag

SEQ ID NO: 4 - DNA, 1635 bp, Clostridium auotethanogenum
atggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccatatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaa

SEQ ID NO: 5 - DNA, 479 bp, Clostridium autoethanogenum
ggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccat

SEQ ID NO: 6 - DNA, 28 bp, Artificial sequence
  synthetic primer
gggttcatatgaaaattagaccacttgg

SEQ ID NO: 7 - DNA, 30 bp, Artificial sequence
  synthetic primer
tcccatgttttcataaggatcttctaattc

SEQ ID NO: 8 - DNA, 30 bp, Artificial sequence
  synthetic primer
attagaagatccttatgaaaacatgggagc

SEQ ID NO: 9 - DNA, 30 bp, Artificial sequence
  synthetic primer
cttagaattccttttgaattagtacattcc

SEQ ID NO: 10 - DNA, 30 bp, Artificial sequence
  synthetic primer
aagcggccgcaaaatagttgataataatgc

SEQ ID NO: 11 - DNA, 30 bp, Artificial sequence
  synthetic primer
tacgcatatgaattcctctccttttcaagc

SEQ ID NO: 12 - DNA, 1978 bp, Clostridium autoethanogenum
atgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtagttaattgaaaaagaaaaataagtatctatataacggttagttgtaaggagggttttttatggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccttatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaa

SEQ ID NO: 13 - DNA, 16 bp, Artificial sequence
  synthetic primer
gtaaaacgacggccag

SEQ ID NO: 14 - DNA, 17 bp, Artificial sequence
  synthetic primer
caggaaacagctatgac

SEQ ID NO: 15 - DNA, 2963 bp, Escherichia coli
cctgcaggataaaaaaattgtagataaattttataaaatagttttatctacaatttttttatcaggaaacagctatgaccgcggccgctgtatccatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccc

SEQ ID NO: 16 - DNA, 5935 bp, Escherichia coli
cctgcaggataaaaaaattgtagataaattttataaaatagttttatctacaatttttttatcaggaaacagctatgaccgcggccgctgtatccatatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaaacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 17 - DNA, 5512 bp, Artificial sequence
  synthetic plasmid
ggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccatatgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtagttaattgaaaaagaaaaataagtatctatataacggttagttgtaaggagggttttttatggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccttatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaattcaaaaggaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgc

SEQ ID NO: 18 - protein, 601 aa, Artificial sequence
  synthetic protein
MFPCNAYIEYGDKNMNSFIEDVEQIYNFIKKNIDVEEKMHFIETYKQKSNMKKEISFSEEYYKQKIMNGKNGVVYTPPEMAAFMVKNLINVNDVIGNPFIKIIDPSCGSGNLICKCFLYLNRIFIKNIEVINSKNNLNLKLEDISYHIVRNNLFGFDIDETAIKVLKIDLFLISNQFSEKNFQVKDFLVENIDRKYDVFIGNPPYIGHKSVDSSYSYVLRKIYGSIYRDKGDISYCFFQKSLKCLKEGGKLVFVTSRYFCESCSGKELRKFLIENTSIYKIIDFYGIRPFKRVGIDPMIIFLVRTKNWNNNIEIIRPNKIEKNEKNKFLDSLFLDKSEKCKKFSISQKSINNDGWVFVDEVEKNIIDKIKEKSKFILKDICHSCQGIITGCDRAFIVDRDIINSRKIELRLIKPWIKSSHIRKNEVIKGEKFIIYSNLIENETECPNAIKYIEQYKKRLMERRECKKGTRKWYELQWGRKPEIFEEKKIVFPYKSCDNRFALDKGSYFSADIYSLVLKKNVPFTYEILLNILNSPLYEFYFKTFAKKLGENLYEYYPNNLMKLCIPSIDFGGENNIEKKLYDFFGLTDKEIEIVEKIKDNC

SEQ ID NO: 19 - DNA, 4709 bp, Artificial sequence
  synthetic plasmid
gtttgccacctgacgtctaagaaaaggaatattcagcaatttgcccgtgccgaagaaaggcccacccgtgaaggtgagccagtgagttgattgctacgtaattagttagttagcccttagtgactcgtaatacgactcactatagggctcgaggcggccgcgcaacgcaattaatgtgagttagctcactcattaggcaccccaggctttacactttatgcttccggctcgtatgttgtgtggaattgtgagcggataacaatttcacacaggaaacacatatgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaagaattcgatatcacccgggaactagtctgcagccctttagtgagggttaattggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagatggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtttactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgc

SEQ ID NO: 20 - DNA, 18 bp, Artificial sequence
  synthetic primer
tttgtaattaagaaggag

SEQ ID NO: 21 - DNA, 18 bp, Artificial sequence
  synthetic primer
gtagaatccttcttcaac

SEQ ID NO: 22 - DNA, 37 bp, Artificial sequence
  synthetic primer
ccgaattcgtcgacaacagagtttgatcctggctcag

SEQ ID NO: 23 - DNA, 37 bp, Artificial sequence
  synthetic primer
cccgggatccaagcttacggctaccttgttacgactt

SEQ ID NO: 24 - DNA, 498 bp, Clostridium autoethanogenum
gagcggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaacatatggaa

SEQ ID NO: 25 - DNA, 563 bp, Clostridium autoethanogenum
ggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggagggaattattca

SEQ ID NO: 26 - DNA, 120 bp, Clostridium autoethanogenum
acagataaaaaaaatatataatacagaagaaaaaattataaatttgtggtataatataaagtatagtaatttaagtttaaaactcgtgaaaacgctaacaaataataggaggtgtattat

SEQ ID NO: 27 - DNA, 350 bp, Clostridium autoethanogenum
acagacaatatagtaatatatgatgttaaaatatcaatatatggttaaaaatctgtatattttttcccattttaattatttgtactataatattacactgagtgtattgtatatttaaaaaatatttggtacaattagttagttaaataaattctaaattgtaaattatcagaatccttattaaggaaatacatagatttaaggagaaatcataaaaaggtgtaatataaactggctaaaattgagcaaaaattgagcaattaagactttttgattgtatctttttatatatttaaggtatataatcttatttatattgggggaacttgatgaataaacatattctagac

SEQ ID NO: 28 - DNA, 1806 bp, Artificial sequence
  synthetic gene
atgtttccgtgcaatgcctatatcgaatatggtgataaaaatatgaacagctttatcgaagatgtggaacagatctacaacttcattaaaaagaacattgatgtggaagaaaagatgcatttcattgaaacctataaacagaaaagcaacatgaagaaagagattagctttagcgaagaatactataaacagaagattatgaacggcaaaaatggcgttgtgtacaccccgccggaaatggcggcctttatggttaaaaatctgatcaacgttaacgatgttattggcaatccgtttattaaaatcattgacccgagctgcggtagcggcaatctgatttgcaaatgttttctgtatctgaatcgcatctttattaagaacattgaggtgattaacagcaaaaataacctgaatctgaaactggaagacatcagctaccacatcgttcgcaacaatctgtttggcttcgatattgacgaaaccgcgatcaaagtgctgaaaattgatctgtttctgatcagcaaccaatttagcgagaaaaatttccaggttaaagactttctggtggaaaatattgatcgcaaatatgacgtgttcattggtaatccgccgtatatcggtcacaaaagcgtggacagcagctacagctacgtgctgcgcaaaatctacggcagcatctaccgcgacaaaggcgatatcagctattgtttctttcagaagagcctgaaatgtctgaaggaaggtggcaaactggtgtttgtgaccagccgctacttctgcgagagctgcagcggtaaagaactgcgtaaattcctgatcgaaaacacgagcatttacaagatcattgatttttacggcatccgcccgttcaaacgcgtgggtatcgatccgatgattatttttctggttcgtacgaagaactggaacaataacattgaaattattcgcccgaacaagattgaaaagaacgaaaagaacaaattcctggatagcctgttcctggacaaaagcgaaaagtgtaaaaagtttagcattagccagaaaagcattaataacgatggctgggttttcgtggacgaagtggagaaaaacattatcgacaaaatcaaagagaaaagcaagttcattctgaaagatatttgccatagctgtcaaggcattatcaccggttgtgatcgcgcctttattgtggaccgtgatatcatcaatagccgtaagatcgaactgcgtctgattaaaccgtggattaaaagcagccatatccgtaagaatgaagttattaagggcgaaaaattcatcatctatagcaacctgattgagaatgaaaccgagtgtccgaatgcgattaaatatatcgaacagtacaagaaacgtctgatggagcgccgcgaatgcaaaaagggcacgcgtaagtggtatgaactgcaatggggccgtaaaccggaaatcttcgaagaaaagaaaattgttttcccgtataaaagctgtgacaatcgttttgcactggataagggtagctattttagcgcagacatttatagcctggttctgaagaaaaatgtgccgttcacctatgagatcctgctgaatatcctgaatagcccgctgtacgagttttactttaagaccttcgcgaaaaagctgggcgagaatctgtacgagtactatccgaacaacctgatgaagctgtgcatcccgagcatcgatttcggcggtgagaacaatattgagaaaaagctgtatgatttctttggtctgacggataaagaaattgagattgtggagaagatcaaagataactgctaa

SEQ ID NO: 29 - DNA, 20 bp, Clostridium autoethanogenum
tcaggaccttctggaactgg

SEQ ID NO: 30 - DNA, 20 bp, Clostridium autoethanogenum
acctccccttttcttggaga

SEQ ID NO: 31 - DNA, 20 bp, Clostridium autoethanogenum
caggtttcggtgctgaccta

SEQ ID NO: 32 - DNA, 20 bp, Clostridium autoethanogenum
aactccgccgttgtatttca

SEQ ID NO: 33 - DNA, 25 bp, Clostridium autoethanogenum
aactacgaagagcggtattgtttta

SEQ ID NO: 34 - DNA, 25 bp, Clostridium autoethanogenum
acttcttttccatctactgttccac

SEQ ID NO: 35 - DNA, 651 bp, Clostridium autoethanogenum
atgttaaaggataaaggtgataatgaaaaagaccttaatgaagaatgtgaaaatgattcagaaaatgaaaaaaaagataaagataatgaaaatgtaaatgaaagcacagaggataattcagaagaagaagtagaagaaacagaagataaagaagataaagaagataaagagataagtttgctaggagaattaaaaaaagaaaattcaaaattaaaagatgaaaataaaaaggccataaatgaattggattctattaaagatagacttgcaagggttatggcagagtatgataactttagaaaaagaactgttaaagagaaggacaatatttattccgatgcttgtaaggatatattaaaagaagttttaccagtgttagataacctggaaagggcagtaaatgtagaaggaaatgcagaagatttgaaaaaaggtatagagatgacaatgaaacaatttaataatgccctttcaaaattaaatgtagaggaaattccttgcgaaggagaatttgatccaaatctacataatgcagttatgcatatagaagatgataaatatgataaaaattctatagtagaagtgttgcaaaaaggatacaaaagagaagacaaaataatcagatacagcatggttaaagtagcaaattaa

SEQ ID NO: 36 - protein, 216 aa, Clostridium autoethanogenum
MLKDKGDNEKDLNEECENDSENEKKDKDNENVNESTEDNSEEEVEETEDKEDKEDKEISLLGELKKENSKLKDENKKAINELDSIKDRLARVMAEYDNFRKRTVKEKDNIYSDACKDILKEVLPVLDNLERAVNVEGNAEDLKKGIEMTMKQFNNALSKLNVEEIPCEGEFDPNLHNAVMHIEDDKYDKNSIVEVLQKGYKREDKIIRYSMVKVAN

SEQ ID NO: 37 - DNA, 1878 bp, Clostridium autoethanogenum
atgtcaaaaataataggtattgatttaggaacaactaattcatgtgttgcagttatggaaggtggagatcctgcagttatagcaaattcagaaggagcaagaacaactccatcagtagtatcattccaggcaaatggagaaagattggtaggtcaagttgccaaaagacaggcaataacaaatcctgataagacaataatgtcaataaaaaggcaaatgggaacagaccataaagtaaatatagatggaaaagattatacaccacaggagatatctgcgatgatactccaaaaaataaaagcagatgctgaagcttatttaggagaaactgtaactgaagcagttataacagtaccagcatattttaacgatagtcagagacaggcaactaaagatgcaggtaagattgcaggattaaatgtacgtagaataataaatgaaccaacagctgcatcacttgcttatggacttgataaaactgatacaagtcaaaagatatttgtatatgacttaggtggaggtacttttgatgtatccatactagaacttggagatggagtatttgaagttaaagctacaaatggtgatactcatctaggtggagatgactttgaccagaaagttatggactatatagcagaagatttcaaagctaagaatggtatagatttaagaaatgacaaaatggcacttcaaagattaaaggaagcagctgaaaaagcaaaaattgaactttcggcatctactcaaacaaatataaacttaccatttattacagcagatgcaactggtccaaaacatatagatatgaatttgacaagagcaaaatttaatgagttgactcaagatctagttgaaagaacaattgaacctatgagaaaagcattaaatgatgcaggacttacaataaatgatataaataagatcatattagttggtggttctacaagaataccagctgttcaggaagcagttaagaattttactggtaaagatccatcaaagggagttaaccctgatgaatgtgtagctgtaggggctgcaattcaggccggagttttaactggagatgtaaaagacgtattactccttgatgttacacctcttacacttggaattgaaactttaggaggagttgccactccacttattgatagaaatactacagtaccaactaagaagagtcaggtattttcaactgcagcagatggccagacttcagttgaaattcatgtagttcaaggtgaaagaaagatggctgctgataataaaactcttggaagatttacgctttcaggaatagctccagctccaaggggaattcctcaaattgaagttacatttgacatagatgccaacggtatagtaaatgtatctgctaaagataaaggaacaggaaaagaagctaatataacaattacagcttcaactaatttaagcgatgatgaaataaacaaggcagtagatgaagctaaaaagtttgaagaacaggataaaaagagaaaagaatccatagacataaaaaataatgcagatcaatctgtatatcagacagaaaagacattaaaggacttaggagataaagtatcagctgaagataagaaaactgtagaggaaaaaattgaagctttaaagaagataaaagatggagaagatttagaggcaataaagaaagctactgaagatttaactcaaactttctatggaattacatctaaaatatatagtcagaatgctcaagcaggacaaaatccaggagcagatccaaatatgggagcaggacaaaatccaggggcaggagcaggttctcaaggtgcatcagaaaaaaaagatgataatgtagttgatgcggattacaaagtagatgatgataaataa

SEQ ID NO: 38 - protein, 625 aa, Clostridium autoethanogenum
MSKIIGIDLGTTNSCVAVMEGGDPAVIANSEGARTTPSVVSFQANGERLVGQVAKRQAITNPDKTIMSIKRQMGTDHKVNIDGKDYTPQEISAMILQKIKADAEAYLGETVTEAVITVPAYFNDSQRQATKDAGKIAGLNVRRIINEPTAASLAYGLDKTDTSQKIFVYDLGGGTFDVSILELGDGVFEVKATNGDTHLGGDDFDQKVMDYIAEDFKAKNGIDLRNDKMALQRLKEAAEKAKIELSASTQTNINLPFITADATGPKHIDMNLTRAKFNELTQDLVERTIEPMRKALNDAGLTINDINKIILVGGSTRIPAVQEAVKNFTGKDPSKGVNPDECVAVGAAIQAGVLTGDVKDVLLLDVTPLTLGIETLGGVATPLIDRNTTVPTKKSQVFSTAADGQTSVEIHVVQGERKMAADNKTLGRFTLSGIAPAPRGIPQIEVTFDIDANGIVNVSAKDKGTGKEANITITASTNLSDDEINKAVDEAKKFEEQDKKRKESIDIKNNADQSVYQTEKTLKDLGDKVSAEDKKTVEEKIEALKKIKDGEDLEAIKKATEDLTQTFYGITSKIYSQNAQAGQNPGADPNMGAGQNPGAGAGSQGASEKKDDNVVDADYKVDDDK

SEQ ID NO: 39 - DNA, 1149 bp, Clostridium autoethanogenum
atggcacagaaggactattatgaagtacttggacttgaaaaaggtgcaagtgatggagatataaaaaaagcatttagaaaattagcattgaaataccacccagataggaaccccaatgataaaaaagctgaagaaaaatttaaggaaataaatgaagcctatcaagtactctcagatcctcagaaaaaggcacaatatgatcagtttggaacaactgacttcaatggcggcggtgatgcaggctttggaggctttggaggttttgatttttcagacatgggaggctttggagatatattcgattctttctttggtggtggaggcggatttggctctagcagcagaagaagaaatgcaccacaaaaaggagcagatcttgaatatactctaaatttaacttttgaagaagctgtttttggagtggaaaaggaaataaatatagctagaaatgaaaaatgtgaggcttgtggtggaacaggagctaaaaaaggaacacatccccatacttgtgataaatgcggtggaacaggacagatgagaactcagaggaatacgcctcttggaagctttgtaagtatgagcacttgtgataaatgtggtggaagaggaactataataaaagatccttgtccagaatgcagaggaaaaggtgcagtaagaaaacatagaaagataaaagtgaaggttccagcaggagtagataatggaaatataattccattaaggggacaaggagaaagtggcaagaacggtggacagtcaggagatctttatgtaaatataagggtttcacctcattctaagtttaagagaaagggatttgatatatatacagatacacatataagctttggtaaagcttcccttggaactagtttaaaagttgcaactatagatggggatgtaaagtatgatgtaccatcaggaactcaatcaggaactgtgtttagacttaaaggcaagggtgtccctagggttaatggtcatggtagaggtgaccaatatgtaaatgtaattgttgatgtacctaaggatttaaatgaaaagcagagagaagccattataatgcttatggaggcaagtggagaaatacctgcaggagaaagtggaaaaaaatctatctttgataaacttaaacatcaccactaa

SEQ ID NO: 40 - protein, 382 aa, Clostridium autoethanogenum
MAQKDYYEVLGLEKGASDGDIKKAFRKLALKYHPDRNPNDKKAEEKFKEINEAYQVLSDPQKKAQYDQFGTTDFNGGGDAGFGGFGGFDFSDMGGFGDIFDSFFGGGGGFGSSSRRRNAPQKGADLEYTLNLTFEEAVFGVEKEINIARNEKCEACGGTGAKKGTHPHTCDKCGGTGQMRTQRNTPLGSFVSMSTCDKCGGRGTIIKDPCPECRGKGAVRKHRKIKVKVPAGVDNGNIIPLRGQGESGKNGGQSGDLYVNIRVSPHSKFKRKGFDIYTDTHISFGKASLGTSLKVATIDGDVKYDVPSGTQSGTVFRLKGKGVPRVNGHGRGDQYVNVIVDVPKDLNEKQREAIIMLMEASGEIPAGESGKKSIFDKLKHHH

SEQ ID NO: 41 - DNA, 4043 bp, Clostridium autoethanogenum
atgttaaaggataaaggtgataatgaaaaagaccttaatgaagaatgtgaaaatgattcagaaaatgaaaaaaaagataaagataatgaaaatgtaaatgaaagcacagaggataattcagaagaagaagtagaagaaacagaagataaagaagataaagaagataaagagataagtttgctaggagaattaaaaaaagaaaattcaaaattaaaagatgaaaataaaaaggccataaatgaattggattctattaaagatagacttgcaagggttatggcagagtatgataactttagaaaaagaactgttaaagagaaggacaatatttattccgatgcttgtaaggatatattaaaagaagttttaccagtgttagataacctggaaagggcagtaaatgtagaaggaaatgcagaagatttgaaaaaaggtatagagatgacaatgaaacaatttaataatgccctttcaaaattaaatgtagaggaaattccttgcgaaggagaatttgatccaaatctacataatgcagttatgcatatagaagatgataaatatgataaaaattctatagtagaagtgttgcaaaaaggatacaaaagagaagacaaaataatcagatacagcatggttaaagtagcaaattaagtttaaaacatacaaattaaatttgtttgaattaaatatatataagataattttaacgcagttaaatttaggaggtaagttaatatgtcaaaaataataggtattgatttaggaacaactaattcatgtgttgcagttatggaaggtggagatcctgcagttatagcaaattcagaaggagcaagaacaactccatcagtagtatcattccaggcaaatggagaaagattggtaggtcaagttgccaaaagacaggcaataacaaatcctgataagacaataatgtcaataaaaaggcaaatgggaacagaccataaagtaaatatagatggaaaagattatacaccacaggagatatctgcgatgatactccaaaaaataaaagcagatgctgaagcttatttaggagaaactgtaactgaagcagttataacagtaccagcatattttaacgatagtcagagacaggcaactaaagatgcaggtaagattgcaggattaaatgtacgtagaataataaatgaaccaacagctgcatcacttgcttatggacttgataaaactgatacaagtcaaaagatatttgtatatgacttaggtggaggtacttttgatgtatccatactagaacttggagatggagtatttgaagttaaagctacaaatggtgatactcatctaggtggagatgactttgaccagaaagttatggactatatagcagaagatttcaaagctaagaatggtatagatttaagaaatgacaaaatggcacttcaaagattaaaggaagcagctgaaaaagcaaaaattgaactttcggcatctactcaaacaaatataaacttaccatttattacagcagatgcaactggtccaaaacatatagatatgaatttgacaagagcaaaatttaatgagttgactcaagatctagttgaaagaacaattgaacctatgagaaaagcattaaatgatgcaggacttacaataaatgatataaataagatcatattagttggtggttctacaagaataccagctgttcaggaagcagttaagaattttactggtaaagatccatcaaagggagttaaccctgatgaatgtgtagctgtaggggctgcaattcaggccggagttttaactggagatgtaaaagacgtattactccttgatgttacacctcttacacttggaattgaaactttaggaggagttgccactccacttattgatagaaatactacagtaccaactaagaagagtcaggtattttcaactgcagcagatggccagacttcagttgaaattcatgtagttcaaggtgaaagaaagatggctgctgataataaaactcttggaagatttacgctttcaggaatagctccagctccaaggggaattcctcaaattgaagttacatttgacatagatgccaacggtatagtaaatgtatctgctaaagataaaggaacaggaaaagaagctaatataacaattacagcttcaactaatttaagcgatgatgaaataaacaaggcagtagatgaagctaaaaagtttgaagaacaggataaaaagagaaaagaatccatagacataaaaaataatgcagatcaatctgtatatcagacagaaaagacattaaaggacttaggagataaagtatcagctgaagataagaaaactgtagaggaaaaaattgaagctttaaagaagataaaagatggagaagatttagaggcaataaagaaagctactgaagatttaactcaaactttctatggaattacatctaaaatatatagtcagaatgctcaagcaggacaaaatccaggagcagatccaaatatgggagcaggacaaaatccaggggcaggagcaggttctcaaggtgcatcagaaaaaaaagatgataatgtagttgatgcggattacaaagtagatgatgataaataatatttcctcttcacgattatataataagtgtgtataatggtaatagttaagggatgagtttttatactcttcccttaatttaagtagagaacccaaatctccgatttggcgtgaatcacttactcatttgaccgaagggaaaaggagttacaaaaattagaacccaaatcttcgatttggtgttaatcacttactcattcgaccgaagggagaaggagttacagaaattagaacttaaattttagtttaatgaaaatattttaggtggtgaaaagtaaaaaatggcacagaaggactattatgaagtacttggacttgaaaaaggtgcaagtgatggagatataaaaaaagcatttagaaaattagcattgaaataccacccagataggaaccccaatgataaaaaagctgaagaaaaatttaaggaaataaatgaagcctatcaagtactctcagatcctcagaaaaaggcacaatatgatcagtttggaacaactgacttcaatggcggcggtgatgcaggctttggaggctttggaggttttgatttttcagacatgggaggctttggagatatattcgattctttctttggtggtggaggcggatttggctctagcagcagaagaagaaatgcaccacaaaaaggagcagatcttgaatatactctaaatttaacttttgaagaagctgtttttggagtggaaaaggaaataaatatagctagaaatgaaaaatgtgaggcttgtggtggaacaggagctaaaaaaggaacacatccccatacttgtgataaatgcggtggaacaggacagatgagaactcagaggaatacgcctcttggaagctttgtaagtatgagcacttgtgataaatgtggtggaagaggaactataataaaagatccttgtccagaatgcagaggaaaaggtgcagtaagaaaacatagaaagataaaagtgaaggttccagcaggagtagataatggaaatataattccattaaggggacaaggagaaagtggcaagaacggtggacagtcaggagatctttatgtaaatataagggtttcacctcattctaagtttaagagaaagggatttgatatatatacagatacacatataagctttggtaaagcttcccttggaactagtttaaaagttgcaactatagatggggatgtaaagtatgatgtaccatcaggaactcaatcaggaactgtgtttagacttaaaggcaagggtgtccctagggttaatggtcatggtagaggtgaccaatatgtaaatgtaattgttgatgtacctaaggatttaaatgaaaagcagagagaagccattataatgcttatggaggcaagtggagaaatacctgcaggagaaagtggaaaaaaatctatctttgataaacttaaacatcaccactaa

SEQ ID NO: 42 - DNA, 30 bp, Artificial sequence
  synthetic primer
gccatatgttaaaggataaaggtgataatg

SEQ ID NO: 43 - DNA, 28 bp, Artificial sequence
  synthetic primer
ccgagctctattagtggtgatgtttaag

SEQ ID NO: 44 - DNA, 29 bp, Artificial sequence
  synthetic primer
aagcggccgcagatagtcataatagttcc

SEQ ID NO: 45 - DNA, 29 bp, Artificial sequence
  synthetic primer
ttccatatgaataattccctccttaaagc

SEQ ID NO: 46 - DNA, 4929 bp, Artificial sequence
  synthetic plasmid
ataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtc

SEQ ID NO: 47 - DNA, 8965 bp, Artificial sequence
  synthetic primer
tccatcaagaagagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgcaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccatatgttaaaggataaaggtgataatgaaaaagaccttaatgaagaatgtgaaaatgattcagaaaatgaaaaaaaagataaagataatgaaaatgtaaatgaaagcacagaggataattcagaagaagaagtagaagaaacagaagataaagaagataaagaagataaagagataagtttgctaggagaattaaaaaaagaaaattcaaaattaaaagatgaaaataaaaaggccataaatgaattggattctattaaagatagacttgcaagggttatggcagagtatgataactttagaaaaagaactgttaaagagaaggacaatatttattccgatgcttgtaaggatatattaaaagaagttttaccagtgttagataacctggaaagggcagtaaatgtagaaggaaatgcagaagatttgaaaaaaggtatagagatgacaatgaaacaatttaataatgccctttcaaaattaaatgtagaggaaattccttgcgaaggagaatttgatccaaatctacataatgcagttatgcatatagaagatgataaatatgataaaaattctatagtagaagtgttgcaaaaaggatacaaaagagaagacaaaataatcagatacagcatggttaaagtagcaaattaagtttaaaacatacaaattaaatttgtttgaattaaatatatataagataattttaacgcagttaaatttaggaggtaagttaatatgtcaaaaataataggtattgatttaggaacaactaattcatgtgttgcagttatggaaggtggagatcctgcagttatagcaaattcagaaggagcaagaacaactccatcagtagtatcattccaggcaaatggagaaagattggtaggtcaagttgccaaaagacaggcaataacaaatcctgataagacaataatgtcaataaaaaggcaaatgggaacagaccataaagtaaatatagatggaaaagattatacaccacaggagatatctgcgatgatactccaaaaaataaaagcagatgctgaagcttatttaggagaaactgtaactgaagcagttataacagtaccagcatattttaacgatagtcagagacaggcaactaaagatgcaggtaagattgcaggattaaatgtacgtagaataataaatgaaccaacagctgcatcacttgcttatggacttgataaaactgatacaagtcaaaagatatttgtatatgacttaggtggaggtacttttgatgtatccatactagaacttggagatggagtatttgaagttaaagctacaaatggtgatactcatctaggtggagatgactttgaccagaaagttatggactatatagcagaagatttcaaagctaagaatggtatagatttaagaaatgacaaaatggcacttcaaagattaaaggaagcagctgaaaaagcaaaaattgaactttcggcatctactcaaacaaatataaacttaccatttattacagcagatgcaactggtccaaaacatatagatatgaatttgacaagagcaaaatttaatgagttgactcaagatctagttgaaagaacaattgaacctatgagaaaagcattaaatgatgcaggacttacaataaatgatataaataagatcatattagttggtggttctacaagaataccagctgttcaggaagcagttaagaattttactggtaaagatccatcaaagggagttaaccctgatgaatgtgtagctgtaggggctgcaattcaggccggagttttaactggagatgtaaaagacgtattactccttgatgttacacctcttacacttggaattgaaactttaggaggagttgccactccacttattgatagaaatactacagtaccaactaagaagagtcaggtattttcaactgcagcagatggccagacttcagttgaaattcatgtagttcaaggtgaaagaaagatggctgctgataataaaactcttggaagatttacgctttcaggaatagctccagctccaaggggaattcctcaaattgaagttacatttgacatagatgccaacggtatagtaaatgtatctgctaaagataaaggaacaggaaaagaagctaatataacaattacagcttcaactaatttaagcgatgatgaaataaacaaggcagtagatgaagctaaaaagtttgaagaacaggataaaaagagaaaagaatccatagacataaaaaataatgcagatcaatctgtatatcagacagaaaagacattaaaggacttaggagataaagtatcagctgaagataagaaaactgtagaggaaaaaattgaagctttaaagaagataaaagatggagaagatttagaggcaataaagaaagctactgaagatttaactcaaactttctatggaattacatctaaaatatatagtcagaatgctcaagcaggacaaaatccaggagcagatccaaatatgggagcaggacaaaatccaggggcaggagcaggttctcaaggtgcatcagaaaaaaaagatgataatgtagttgatgcggattacaaagtagatgatgataaataatatttcctcttcacgattatataataagtgtgtataatggtaatagttaagggatgagtttttatactcttcccttaatttaagtagagaacccaaatctccgatttggcgtgaatcacttactcatttgaccgaagggaaaaggagttacaaaaattagaacccaaatcttcgatttggtgttaatcacttactcattcgaccgaagggagaaggagttacagaaattagaacttaaattttagtttaatgaaaatattttaggtggtgaaaagtaaaaaatggcacagaaggactattatgaagtacttggacttgaaaaaggtgcaagtgatggagatataaaaaaagcatttagaaaattagcattgaaataccacccagataggaaccccaatgataaaaaagctgaagaaaaatttaaggaaataaatgaagcctatcaagtactctcagatcctcagaaaaaggcacaatatgatcagtttggaacaactgacttcaatggcggcggtgatgcaggctttggaggctttggaggttttgatttttcagacatgggaggctttggagatatattcgattctttctttggtggtggaggcggatttggctctagcagcagaagaagaaatgcaccacaaaaaggagcagatcttgaatatactctaaatttaacttttgaagaagctgtttttggagtggaaaaggaaataaatatagctagaaatgaaaaatgtgaggcttgtggtggaacaggagctaaaaaaggaacacatccccatacttgtgataaatgcggtggaacaggacagatgagaactcagaggaatacgcctcttggaagctttgtaagtatgagcacttgtgataaatgtggtggaagaggaactataataaaagatccttgtccagaatgcagaggaaaaggtgcagtaagaaaacatagaaagataaaagtgaaggttccagcaggagtagataatggaaatataattccattaaggggacaaggagaaagtggcaagaacggtggacagtcaggagatctttatgtaaatataagggtttcacctcattctaagtttaagagaaagggatttgatatatatacagatacacatataagctttggtaaagcttcccttggaactagtttaaaagttgcaactatagatggggatgtaaagtatgatgtaccatcaggaactcaatcaggaactgtgtttagacttaaaggcaagggtgtccctagggttaatggtcatggtagaggtgaccaatatgtaaatgtaattgttgatgtacctaaggatttaaatgaaaagcagagagaagccattataatgcttatggaggcaagtggagaaatacctgcaggagaaagtggaaaaaaatctatctttgataaacttaaacatcaccactaatagagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgc

SEQ ID NO: 48 - DNA, 5024 bp, Artificial sequence
  synthetic plasmid
gagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaatagggttttaggtaaaattaagttattttaagaagtaattacaataaaaattgaagttattgctttaaggagggaattattcatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaa

SEQ ID NO: 49 - DNA, 2598 bp, Clostridium autoethanogenum
atgaacatagataaacttacaataaaagttcaaaatgcaatgaatgaagcacaacttacagcagtgagatataatcatcaacaggtagatgtgattcatatgttttcagctttagtgtttgagcaagatggacttattccaaatatatttggaaagatgtctgtaaatttaaaatctctggtaaaggaaactaaagatgtattagacaagatgcctaaagtgctgggagaaggagcacaaagttcctccgtctatgcaactagaagatttgaagatgtttttttgcaagcagaaaagatagctcaaaaattcaaagattcatatataagtgtagagcacgtaatgcttggtatcatggaagttcactcttctgacgtagacggtatacttaagaaatttgatattacaaaggatgcatttttagaagccttgtctcaagtaaggggaaatcaaagagttgaaactcaggatccagagggaacttatgaggcacttgcaaagtatgggagaaatcttgtggaagaggcaaaaaaacataaacttgatccggttataggtagagatgaagaaataagaagagttgttagaatactttcaagaagaactaaaaacaatcctgtactaataggtgatccaggggtaggaaaaactgctataattgaaggtttggcagagagaattgtaagaggagatataccagaaggcttaaaaaataagataatattttcactagatatgggagcattaattgctggtgccaaatttagaggagaatttgaagaaagattaaaggccgtattaaaagaagtacagaaaagcgaaggaaaaatagtgctttttatagatgaaattcacaccatagttggagctggaaaaacagaaggttctatggatgctggaaatttaataaaaccaatgctagcaagaggtgaattgcactgcataggggcaactacttttgatgaatatagaaaatatatagaaaaagataaggctttagagagaagatttcaaccggtggttatagatgaacctactgtagaggattctatctctatacttagggggcttaaggaaaagtttgaaatatatcatggtataagaattcatgattctgctattgtggctgccgcaaagctgtcagatagatatataacagatagataccttccagataaggctatagatttaattgatgaagcttgtgctatgataagaactgaaattgacagcatgccaactgaaatggataatgttaaaagaaaaatatttcagcttgagattgaaaaagaggcgctttccaaagaaaaagacactgcttcaatggaaagacttaaagcagtagaaaaggaacttagcaatcttaaagatagagataatgagatgactgctaagtatgaaaaggaaaaggcaaatataactgaggttagaaatttaaagaaacagctggatgaggcaagaggacaaattgaaaaagcagagagagaatatgatttaaataaaattgcagaattaaaatatggtgttattccaaaactggaaagtactatagatgaaaaagagcaatctattaaagaaaacaatgaagctgcaatgttaaaagaagaagttacagaacaagaaatttctcagatagtatctaaatggactggaatacctgtttcaaaattagtagaaggggaaagacagaaattagtaaaattagaagatgaacttgcaaagagagttataggtcagaaggaagcagttacagcagtttcaaatgcggtacttagagcaagagcgggtatgaaagatcctaaaaggccaataggttcttttatatttttaggacctacgggtgtaggtaaaactgaacttgcaaaaactttagcaaggacactgtttgatagtgaagaaaatataataagaatagatatgtcagagtatatggaaaagtattctgtttcgagacttataggagctcctccaggatatgtaggatatgacgaaggaggtcagcttacggaggcagtgagaagaaaaccatatagtgtaatattatttgatgaaatagaaaaggctcatgaagatgtgtttaatatattccttcaaatattagatgatggaaggcttactgacaatcagggtaaggtagttgattttaaaaattctattataattatgacttctaacataggaagcagttatttattacagaacaaaagcagtaatggaatagataaagatgtaagagacaaagtaatgagtgacatgaaatttaagtttaaacctgaatttttaaatagactagatgatataataatgtttaagcccctaaacacagaagaaattaaattcataatagatatattcctaaaggatatagaaaataggcttaaggagaaaaatatatccatacaaataaccccaaaagcaaaagaagttatggcagaagaaggttatgatcctgtttatggagcaaggcctttaaaaagatatatagaaaacattctggaaacatctattgcaaagaagataattaatggagatatatatacaggctgcaaggtaagagtagattatgaaaatgataagtttaaaatagaaaaactataa

SEQ ID NO: 50 - protein, 865 aa, Clostridium autoethanogenum
MNIDKLTIKVQNAMNEAQLTAVRYNHQQVDVIHMFSALVFEQDGLIPNIFGKMSVNLKSLVKETKDVLDKMPKVLGEGAQSSSVYATRRFEDVFLQAEKIAQKFKDSYISVEHVMLGIMEVHSSDVDGILKKFDITKDAFLEALSQVRGNQRVETQDPEGTYEALAKYGRNLVEEAKKHKLDPVIGRDEEIRRVVRILSRRTKNNPVLIGDPGVGKTAIIEGLAERIVRGDIPEGLKNKIIFSLDMGALIAGAKFRGEFEERLKAVLKEVQKSEGKIVLFIDEIHTIVGAGKTEGSMDAGNLIKPMLARGELHCIGATTFDEYRKYIEKDKALERRFQPVVIDEPTVEDSISILRGLKEKFEIYHGIRIHDSAIVAAAKLSDRYITDRYLPDKAIDLIDEACAMIRTEIDSMPTEMDNVKRKIFQLEIEKEALSKEKDTASMERLKAVEKELSNLKDRDNEMTAKYEKEKANITEVRNLKKQLDEARGQIEKAEREYDLNKIAELKYGVIPKLESTIDEKEQSIKENNEAAMLKEEVTEQEISQIVSKWTGIPVSKLVEGERQKLVKLEDELAKRVIGQKEAVTAVSNAVLRARAGMKDPKRPIGSFIFLGPTGVGKTELAKTLARTLFDSEENIIRIDMSEYMEKYSVSRLIGAPPGYVGYDEGGQLTEAVRRKPYSVILFDEIEKAHEDVFNIFLQILDDGRLTDNQGKVVDFKNSIIIMTSNIGSSYLLQNKSSNGIDKDVRDKVMSDMKFKFKPEFLNRLDDIIMFKPLNTEEIKFIIDIFLKDIENRLKEKNISIQITPKAKEVMAEEGYDPVYGARPLKRYIENILETSIAKKIINGDIYTGCKVRVDYENDKFKIEKL

SEQ ID NO: 51 - DNA, 2442 bp, Clostridium autoethanogenum
atgatgtttggaagatttacggaaagagcacaaaaagtattagtttatgctcaagaagaagcacaagcacttcaacatggatatgtaggtacagaacatatacttttgggaatattaaaagaagaaggaatatccaggaatcttttaagtgatatgaatgtaaacattgaaacagtaagaaattttatagaagaatatgagggtaggggagaaataaatttgtacaataaagaaatccctcttactccaaggacaaaaaggcttttagagctaagtttgtttgaagccagaaatctaaaccataactatataagtccagagcatatcctacttgcacttataagagaagcagagggagttgcgtttactattttaaataatttgggagtagattttaataagctgaggaaggaacttgtggattcactttcgggagagcaatcatcaatgaattcaaacagtactaagaaagaaaatggagagccaaccccaactttagatcagtttggaagagacttaacagacatggcaaaagagggaaagttggaccctgttataggaagagataaagaaactcagagggttttggaaatactaagtagaagaactaagaataatccttgtctaataggagaccctggtgtaggtaaaactgcaattgcagaaggattggcagaaaagatagtatcgtgtaatatacctgaacttttaaggggaaaaagagtagtaacattagatctttcttcaatgatagcgggatcaaaatatagaggagaatttgaagaaagacttaaaaaagttatggaagagataagaaaatcaggtaatgtaatattattcatagatgaaattcatactataataggagcgggagcagcagaaggtgctatagatgcatctaatatattaaaaccagctttagctagaggagaaatacaatgcataggagccactactatagatgaatatagaaaatacattgaaaaggatgctgcacttgaaagaagatttcagcctataatagtaggagaacctactaaggaggaggcagttttaatattaaaaggtcttagagataaatatgaggctcatcatagagtaaaaataatagatgaagctatagatgcagcagtaaatttatcagatagatatattacagatagatatttacctgataaggcaattgatcttatagatgaagcagctgctaaagttagaatacaaaatttaattgccccaccagatttaaaaaatttagaagaagaattagaaaaggcaactaaggaaaaagaggattctattagagtacaggattttgaaaaagctgctagtttaagggataaagaaaaggaattaaaaaataagttagaaggattaaaaactaactggaagactaaaaaagaagtatcaacacttacggtaggagaagaccaaattgcatcagtggtatctcagtggaccaatatacctgtagagaagttaactgaaaaagaatcagagagattgctcaaactagaggaaattcttcataagagagtagtaggtcaagatgaagcagttaaatccatttctaaggcagtaagaagggctagagttgggcttaaagatccaaagagacctataggttcatttatatttttggggcctacaggtgttggaaaaactgagttgtcaaaagctttggcagaggctatgtttggcgacgaaaacaatatgataagaattgatatgtcggaatatatggaaaagcatacagtatctagacttataggatcgcctccagggtatgtaggctatgatgaaggaggacagcttactgaaaaagtaagaagaaatccttattcagtagtattgtttgatgaaatagaaaaagcacacccagaagtatttaatatattacttcaaatacttgaagatggaagattaaccgatggaaaaggaaaaacaataaactttaaaaataccataataataatgacttctaatgtaggagcagctaccattagaaaacaaaaatctatgggatttactgctgctaaaggtgatgacaaggaaagtcaatatgaaaagatgaaagataatataatggaggaacttaaaaattcttttagacctgagtttttaaacagaatagatgatattatagtcttccaccagttagaagaggaagatttaaaacaaatagtaaaactcatgttaaaagatgtttcctcaaggcttaaagatcaggaaatagaaattggatttacagataaagcgcaggagcttttggcaaaagaaggctttgatttaacttatggtgcaagaccgcttagaagagcaattacaaaaactgtagaagataaactttctgaagaaatgcttagaggtaatgttaaaaaaggagataaagttaaagtagttgtagaaaaaggagaattatcatttaataaagtcaattaa

SEQ ID NO: 52 - protein, 813 aa, Clostridium autoethanogenum
MMFGRFTERAQKVLVYAQEEAQALQHGYVGTEHILLGILKEEGISRNLLSDMNVNIETVRNFIEEYEGRGEINLYNKEIPLTPRTKRLLELSLFEARNLNHNYISPEHILLALIREAEGVAFTILNNLGVDFNKLRKELVDSLSGEQSSMNSNSTKKENGEPTPTLDQFGRDLTDMAKEGKLDPVIGRDKETQRVLEILSRRTKNNPCLIGDPGVGKTAIAEGLAEKIVSCNIPELLRGKRVVTLDLSSMIAGSKYRGEFEERLKKVMEEIRKSGNVILFIDEIHTIIGAGAAEGAIDASNILKPALARGEIQCIGATTIDEYRKYIEKDAALERRFQPIIVGEPTKEEAVLILKGLRDKYEAHHRVKIIDEAIDAAVNLSDRYITDRYLPDKAIDLIDEAAAKVRIQNLIAPPDLKNLEEELEKATKEKEDSIRVQDFEKAASLRDKEKELKNKLEGLKTNWKTKKEVSTLTVGEDQIASVVSQWTNIPVEKLTEKESERLLKLEEILHKRVVGQDEAVKSISKAVRRARVGLKDPKRPIGSFIFLGPTGVGKTELSKALAEAMFGDENNMIRIDMSEYMEKHTVSRLIGSPPGYVGYDEGGQLTEKVRRNPYSVVLFDEIEKAHPEVFNILLQILEDGRLTDGKGKTINFKNTIIIMTSNVGAATIRKQKSMGFTAAKGDDKESQYEKMKDNIMEELKNSFRPEFLNRIDDIIVFHQLEEEDLKQIVKLMLKDVSSRLKDQEIEIGFTDKAQELLAKEGFDLTYGARPLRRAITKTVEDKLSEEMLRGNVKKGDKVKVVVEKGELSFNKVN

SEQ ID NO: 53 - DNA, 543 bp, Clostridium autoethanogenum
  misc_feature (530)..(539): n is a, c, g, or t
atgagtttagtaccagtagttgtagaacaaacaagtagaggggaaaggtcttacgatatttattctaggcttttaaaagatagaatagtaatgctgagtgaagaggttaatgatgtttctgctagtctggtagtagcacagctgttattcctagaagctgaagaccctgacaaagatatatatctttatataaatagtccaggtggatcaataacctcaggaatggcaatttatgatacaatgcagtatataaagtcagatgtgtccactatatgtataggcatgggagcttctatgggagcatttttgcttacagctggtgcaaagggaaagagatttgcacttccaaacgcagagataatgatacatcaaccacttggaggattccaaggtcaggcaactgatattggaattcatgcaaaaagaatattagacataaagaaaaagttaaatactataataagtgaaagaacagggcagccccttgaaaaagttgaaaaagacactgagagagataactttatgacagnnnnnnnnnnctaa

SEQ ID NO: 54 - protein, 180 aa, Clostridium autoethanogenum
  misc_feature (177)..(180): Xaa can be any naturally occurring amino acid
MSLVPVVVEQTSRGERSYDIYSRLLKDRIVMLSEEVNDVSASLVVAQLLFLEAEDPDKDIYLYINSPGGSITSGMAIYDTMQYIKSDVSTICIGMGASMGAFLLTAGAKGKRFALPNAEIMIHQPLGGFQGQATDIGIHAKRILDIKKKLNTIISERTGQPLEKVEKDTERDNFMTXXXX

SEQ ID NO: 55 - DNA, 447 bp, Clostridium autoethanogenum
atgtttgatatggttccatttagaaaaaacaattctttaaaaagaggcgatgcattcgataactttgtagattcattctttaataatgacttttttgcacctatgaacatgaatggttttggcaatggttttaaagttgatcttaaggaaaatgaaacttcttatatagtttgtgctgatctaccaggaataaataaagattctatagacttagactttaataataactacttgaccatatctgcaaaaagggatgactctatagaagataaaaacgaaaactttgtaagacgtgaaagaagatatggtgaattcagaagaagtttttatattgataatgtagatgacaaaaacattacagcttcttttaatgatggagttttaaaagttatccttccaaaactttcacaaggtaaaagacaaggtaagaaaatagatatacaataa

SEQ ID NO: 56 - protein, 148 aa, Clostridium autoethanogenum
MFDMVPFRKNNSLKRGDAFDNFVDSFFNNDFFAPMNMNGFGNGFKVDLKENETSYIVCADLPGINKDSIDLDFNNNYLTISAKRDDSIEDKNENFVRRERRYGEFRRSFYIDNVDDKNITASFNDGVLKVILPKLSQGKRQGKKIDIQ

SEQ ID NO: 57 - DNA, 1893 bp, Clostridium autoethanogenum
atgaaaggaaatgataaaatggctacaaaacaatttaaggctgaatccaaaagattacttaatttaatgattaattctatttacacaaataaggaaatatttttgagggaacttatatcaaatgccagtgatgcaattgataaaagttattatcgttcactagttgatgaaaatgttagctttaataaagaagacttttatattagaattgcagcagataaagaaaacaaaacccttactattactgatactggtataggtatgacaaaagatgaacttgaaaataatctaggtactattgcaaaaagtggttcctttacatttaaaaatgaaaatgaagcaaaagaaggagtagatatcataggtcaatttggagttggcttttactctgcttttatggtgtcagatttagttactgtaaaaagccgtgccttgaattcagatgaagcatacaaatgggaatcaaagggcgtagaagggtatacaattgagccttgtgaaaaaaatgaagttggaactgaaattacattgaaaattaaagaaagtactgatgacgaaaagtacgacgaatttttagatgaatataaaataagatcattaattaaaaaatactctgattttataaagtatccaattaaaatgatggtaaagaaaagcaaactaaaagaaggcagtaaggatgaacatgaagactattttgaagatgaaactttaaatagtatggttccaatttggagaaaaaataaaaatgaattgaagcctgaagattataatcagttctacatggataaacattttggatatgaaaaacctcttaaggtaatccattcgagtgttgaaggtgttgtaagttataataccttactttttattccagctagagcaccttttgatttctatactaaggaattcgaaaagggattggaactttattcaaatggcgttctcataatggagaaatgtggagatcttttaccagattactttagctttgtacagggattagttgattcagcagatctttcacttaatatttcaagagaacttttgcagcatgatagacagcttaaatttattgcaaaaaagataaaggaaaaaattaaaagtgaacttttattaatgcagaaaaatgatagagaaaagtatgatgagttttataaaaactttggaaaacagcttaaatatggtgtttatgctgattttggaagtaataaagaggtacttcaagatttactcatgttctattcttctacagagaaaaagcttgtaagccttgatgaatatgtttctcgtatgaaggaagaccagaagtttatatactatgcaactggtgaaaacatagataaaattgaaaaattaccacaaactgaagtcgttaaggataaaggatatgaaatattgtactttacagatgaagttgacgaatttgcaattaagatgcttatgaaatacaaggaaaaggaatttaaatctgtttcaagcaaagatttaggatttgaatctgatgacaaggaaagtgaaaaggaaacaaaggaaaataaagaactgtttgatttcatgaaagaaacattaaatggaaaagttaaagaagttagggcatcaaacagattaaagacccatcctgtttgtcttgcaaatggtggtgaattgtcaattgatatggaaaaagtgcttaatacaatgccaaacaatcaaaatgtaaaagcagataaaattttagagataaatacaaatcataaaatgtttaaatcaataaaagatgcatttgaaaatgataaagataaacttaaaatgctttcaaatgtattatataatcaggcacttatgattgaaggactgcctgtagatgatcctgtggaatttgcaaatgatgtttgcagtttgattaaataa

SEQ ID NO: 58 - protein, 630 aa, Clostridium autoethanogenum
MKGNDKMATKQFKAESKRLLNLMINSIYTNKEIFLRELISNASDAIDKSYYRSLVDENVSFNKEDFYIRIAADKENKTLTITDTGIGMTKDELENNLGTIAKSGSFTFKNENEAKEGVDIIGQFGVGFYSAFMVSDLVTVKSRALNSDEAYKWESKGVEGYTIEPCEKNEVGTEITLKIKESTDDEKYDEFLDEYKIRSLIKKYSDFIKYPIKMMVKKSKLKEGSKDEHEDYFEDETLNSMVPIWRKNKNELKPEDYNQFYMDKHFGYEKPLKVIHSSVEGVVSYNTLLFIPARAPFDFYTKEFEKGLELYSNGVLIMEKCGDLLPDYFSFVQGLVDSADLSLNISRELLQHDRQLKFIAKKIKEKIKSELLLMQKNDREKYDEFYKNFGKQLKYGVYADFGSNKEVLQDLLMFYSSTEKKLVSLDEYVSRMKEDQKFIYYATGENIDKIEKLPQTEVVKDKGYEILYFTDEVDEFAIKMLMKYKEKEFKSVSSKDLGFESDDKESEKETKENKELFDFMKETLNGKVKEVRASNRLKTHPVCLANGGELSIDMEKVLNTMPNNQNVKADKILEINTNHKMFKSIKDAFENDKDKLKMLSNVLYNQALMIEGLPVDDPVEFANDVCSLIK

SEQ ID NO: 59 - DNA, 11482 bp, Artificial sequence
  synthetic plasmid
gaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgcaaaatagttgataataatgtagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatccatatgttaaaggataaaggtgataatgaaaaagaccttaatgaagaatgtgaaaatgattcagaaaatgaaaaaaaagataaagataatgaaaatgtaaatgaaagcacagaggataattcagaagaagaagtagaagaaacagaagataaagaagataaagaagataaagagataagtttgctaggagaattaaaaaaagaaaattcaaaattaaaagatgaaaataaaaaggccataaatgaattggattctattaaagatagacttgcaagggttatggcagagtatgataactttagaaaaagaactgttaaagagaaggacaatatttattccgatgcttgtaaggatatattaaaagaagttttaccagtgttagataacctggaaagggcagtaaatgtagaaggaaatgcagaagatttgaaaaaaggtatagagatgacaatgaaacaatttaataatgccctttcaaaattaaatgtagaggaaattccttgcgaaggagaatttgatccaaatctacataatgcagttatgcatatagaagatgataaatatgataaaaattctatagtagaagtgttgcaaaaaggatacaaaagagaagacaaaataatcagatacagcatggttaaagtagcaaattaagtttaaaacatacaaattaaatttgtttgaattaaatatatataagataattttaacgcagttaaatttaggaggtaagttaatatgtcaaaaataataggtattgatttaggaacaactaattcatgtgttgcagttatggaaggtggagatcctgcagttatagcaaattcagaaggagcaagaacaactccatcagtagtatcattccaggcaaatggagaaagattggtaggtcaagttgccaaaagacaggcaataacaaatcctgataagacaataatgtcaataaaaaggcaaatgggaacagaccataaagtaaatatagatggaaaagattatacaccacaggagatatctgcgatgatactccaaaaaataaaagcagatgctgaagcttatttaggagaaactgtaactgaagcagttataacagtaccagcatattttaacgatagtcagagacaggcaactaaagatgcaggtaagattgcaggattaaatgtacgtagaataataaatgaaccaacagctgcatcacttgcttatggacttgataaaactgatacaagtcaaaagatatttgtatatgacttaggtggaggtacttttgatgtatccatactagaacttggagatggagtatttgaagttaaagctacaaatggtgatactcatctaggtggagatgactttgaccagaaagttatggactatatagcagaagatttcaaagctaagaatggtatagatttaagaaatgacaaaatggcacttcaaagattaaaggaagcagctgaaaaagcaaaaattgaactttcggcatctactcaaacaaatataaacttaccatttattacagcagatgcaactggtccaaaacatatagatatgaatttgacaagagcaaaatttaatgagttgactcaagatctagttgaaagaacaattgaacctatgagaaaagcattaaatgatgcaggacttacaataaatgatataaataagatcatattagttggtggttctacaagaataccagctgttcaggaagcagttaagaattttactggtaaagatccatcaaagggagttaaccctgatgaatgtgtagctgtaggggctgcaattcaggccggagttttaactggagatgtaaaagacgtattactccttgatgttacacctcttacacttggaattgaaactttaggaggagttgccactccacttattgatagaaatactacagtaccaactaagaagagtcaggtattttcaactgcagcagatggccagacttcagttgaaattcatgtagttcaaggtgaaagaaagatggctgctgataataaaactcttggaagatttacgctttcaggaatagctccagctccaaggggaattcctcaaattgaagttacatttgacatagatgccaacggtatagtaaatgtatctgctaaagataaaggaacaggaaaagaagctaatataacaattacagcttcaactaatttaagcgatgatgaaataaacaaggcagtagatgaagctaaaaagtttgaagaacaggataaaaagagaaaagaatccatagacataaaaaataatgcagatcaatctgtatatcagacagaaaagacattaaaggacttaggagataaagtatcagctgaagataagaaaactgtagaggaaaaaattgaagctttaaagaagataaaagatggagaagatttagaggcaataaagaaagctactgaagatttaactcaaactttctatggaattacatctaaaatatatagtcagaatgctcaagcaggacaaaatccaggagcagatccaaatatgggagcaggacaaaatccaggggcaggagcaggttctcaaggtgcatcagaaaaaaaagatgataatgtagttgatgcggattacaaagtagatgatgataaataatatttcctcttcacgattatataataagtgtgtataatggtaatagttaagggatgagtttttatactcttcccttaatttaagtagagaacccaaatctccgatttggcgtgaatcacttactcatttgaccgaagggaaaaggagttacaaaaattagaacccaaatcttcgatttggtgttaatcacttactcattcgaccgaagggagaaggagttacagaaattagaacttaaattttagtttaatgaaaatattttaggtggtgaaaagtaaaaaatggcacagaaggactattatgaagtacttggacttgaaaaaggtgcaagtgatggagatataaaaaaagcatttagaaaattagcattgaaataccacccagataggaaccccaatgataaaaaagctgaagaaaaatttaaggaaataaatgaagcctatcaagtactctcagatcctcagaaaaaggcacaatatgatcagtttggaacaactgacttcaatggcggcggtgatgcaggctttggaggctttggaggttttgatttttcagacatgggaggctttggagatatattcgattctttctttggtggtggaggcggatttggctctagcagcagaagaagaaatgcaccacaaaaaggagcagatcttgaatatactctaaatttaacttttgaagaagctgtttttggagtggaaaaggaaataaatatagctagaaatgaaaaatgtgaggcttgtggtggaacaggagctaaaaaaggaacacatccccatacttgtgataaatgcggtggaacaggacagatgagaactcagaggaatacgcctcttggaagctttgtaagtatgagcacttgtgataaatgtggtggaagaggaactataataaaagatccttgtccagaatgcagaggaaaaggtgcagtaagaaaacatagaaagataaaagtgaaggttccagcaggagtagataatggaaatataattccattaaggggacaaggagaaagtggcaagaacggtggacagtcaggagatctttatgtaaatataagggtttcacctcattctaagtttaagagaaagggatttgatatatatacagatacacatataagctttggtaaagcttcccttggaactagtttaaaagttgcaactatagatggggatgtaaagtatgatgtaccatcaggaactcaatcaggaactgtgtttagacttaaaggcaagggtgtccctagggttaatggtcatggtagaggtgaccaatatgtaaatgtaattgttgatgtacctaaggatttaaatgaaaagcagagagaagccattataatgcttatggaggcaagtggagaaatacctgcaggagaaagtggaaaaaaatctatctttgataaacttaaacatcaccactaatagagctcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaatagggttttaggtaaaattaagttattttaagaagtaattacaataaaaattgaagttattgctttaaggagggaattattggatccatgaaaattagaccacttggagacagagttgtaattaaaaaattagaagctgaggaaactacgaagagcggtattgttttaccaggaagtgctaaagaaaaaccacaagaagcagaagttgtggcagtaggaattggtggaacagtagatggaaaagaagttaaaatggaagtaaaagtaggagataaggtattattctccaaatatgctggaaatgaagtaaaaatagatgcacaagagtacactattttaaaacaggacgacatattagctataatcgagtagttaattgaaaaagaaaaataagtatctatataacggttagttgtaaggagggttttttatggcaaaaagtattttatttggtgaagatgcaagaaaatcaatgcaagaaggtgtaaataagctagcaaatgcagtaaaggttacacttggacctaagggaagaaatgtagtacttgataagaaatttggttcaccgcttattacaaatgacggtgttacaatagcaaaggaaatagaattagaagatccttatgaaaacatgggagcacaacttgtaaaagaagttgctacaaagacaaatgatgtagctggagatggaacaactacagctactttacttgctcaagcaataataagagaaggattaaaaaatgttacagctggagcaaatccaatgcttataagacaaggtataaagatggctgtagataaagctgtagaagaaataaaaaaagtttcaacaactgtaaagggaaaagaagatatagcaagaattgcagctatatcagcttctgatgaagaaataggtaaattaatagctgatgccatggaaaaggtaggtaacgaaggtgtcataactgttgaagagtcaaaaactatgggaactgagttagatgtagttgaaggtatgcagtttgacagaggttatttaagtccatatatggttactgattcagaaaaaatggaagctgcaatagaagatccatatatattaataacagacaagaagatatcaaatattcaagatatattaccattacttgagaaaatagttcaacaaggaaagaagttacttataatagctgaagatgtagaaggagaagcacttgcaactttagttgtaaataagttaagaggaacatttacttgtgtagcagtaaaggcacctggatttggtgacagaagaaaagaaatgcttcaggatatagcaatacttactggaggacaggtaatatcagaagaattgggaagagacttaaaagaagctgaattagaggatttaggaagagctgaatctgtaaagatagataaagaaaatactactatagtaaatggacgaggagataagaaagctatagcagatagagtatcccagattaaggttcaaatagaagaaactacttcagattttgataaagaaaaacttcaagaaagacttgcaaaacttgcaggtggagtagctgtagtaaaagttggagcagcaactgaaactgaattaaaagagaaaaaattaagaatagaagatgctttagcagctacaaaagcaggtgttgaagaaggtatgggaccaggaggcggaactgcttatataaatgcaattccagaagttgaaaaattaacttcagatgtaccggatgtaaaagttggtatagacataataagaaaagcattggaagaaccagttagacaaatagcaagcaatgctggtgttgaaggttcagtaataatccaaaaagttagaaatagtgaaattggtgttggatacgatgcattaaaaggcgaatatgtaaacatggtagaaaagggtatagtagacccaactaaggttacaagatcagcacttcaaaatgcagcatccgtagcagctacattcttaactacagaagcagcagttgcagatattccagaaaaagcacctgcaggtccagcagcaggagcaccaggaatgggcggaatggaaggaatgtactaagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcaca

SEQ ID NO: 60 - DNA, 28 bp, Artificial sequence
  synthetic primer
gagcggccgcaatatgatatttatgtcc

SEQ ID NO: 61 - DNA, 28 bp, Artificial sequence
  synthetic primer
ttccatatgtttcatgttcatttcctcc